(19) United States
(12) Patent Application Publication
(10) Pub. No.: US 2002/0072604 A1
(43) Pub. Date: Jun. 13, 2002

(54) Melanocortin receptor ligands

(76) Inventors: Philip A. Carpino, Groton, CT (US); Bridget M. Cole, Stonington, CT (US); Bradley P. Morgan, Lyme, CT (US)

Correspondence Address: Gregg C. Benson, Pfizer Inc., Patent Department, MS 4159, Eastern Point Road, Groton, CT, 06340, US

(21) Appl. No.: 09/891,026

(22) Filed: Jun. 25, 2001

Related U.S. Application Data
(60) Provisional application No. 60/214,616, filed on Jun. 28, 2000.

(51) Int. Cl. C07D 487/02

(52) U.S. Cl. 544/235; 544/278; 544/280; 544/344; 544/350; 546/79; 546/118

(57) ABSTRACT

A compound of the formula

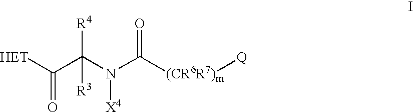

wherein R³, R⁴, R⁶, R⁷, X⁴, Q and HET are as defined above, useful for the treatment or prevention of disorders, diseases or conditions responsive to the activation of melanocortin receptor.

### CROSS-REFERENCE TO RELATED APPLICATION

This non-provisional patent application is based upon and claims priority from U.S. provisional application No. 60/214,616, filed Jun. 28, 2000.

### BACKGROUND OF THE INVENTION

Melanocortins are peptides derived from pro-opiomelanocortins (POMC) that bind to and activate G-protein coupled receptors (GPCR's) of the melanocortin receptor family. These chemical messengers regulate a diverse number of physiological processes including food intake and metabolism.

There are five melanocortin receptors that have been cloned, MCR1, MCR2, MCR3, MCR4, MCR5, and are expressed in various tissue. MCR1 is specifically expressed in melanocytes and melanoma cells, MCR2 is the ACTH receptor and is expressed in adrenal tissue, MCR3 is predominately expressed in the brain and limbic system, MCR4 is widely expressed in the brain and spinal cord, and MCR5 is expressed in the brain and many peripheral tissues including skin, adipose tissue, skeletal muscle, and lymphoid tissue. MCR3 may be involved in the control of food intake and thermogenesis as well as sexual dysfunction. MCR4 inactivation has been shown to cause obesity.

### SUMMARY OF THE INVENTION

The present invention relates to a compound of the formula

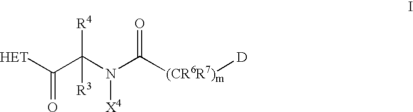

or a stereoisomeric mixture thereof, diastereomerically enriched, diastereomerically pure, enantiomerically enriched or enantiomerically pure isomer thereof, or a prodrug of such compound, mixture or isomer thereof, or a pharmaceutically acceptable salt of the compound, mixture, isomer or prodrug, wherein:

m is 0,1 or 2;

HET is a heterocyclic moiety selected from the group consisting of

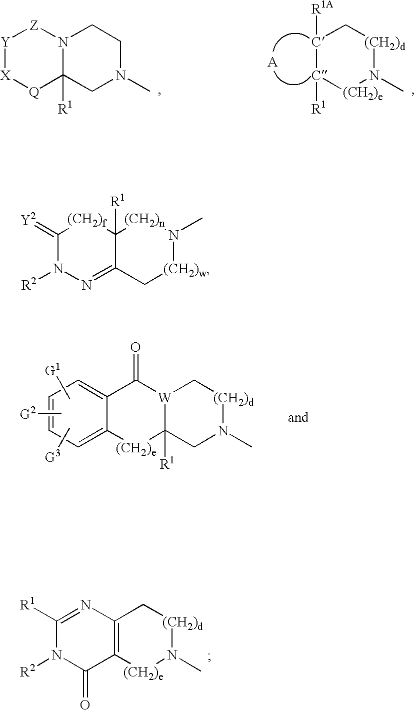

d is 0, 1 or 2;

e is 1 or 2;

f is 0 or 1;

n and w are 0, 1 or 2, provided that n and w cannot both be 0 at the same time;

Y² is oxygen or sulfur;

A is a radical, where the left hand side of the radical as shown below is connected to C″ and the right hand side of the radical as shown below is connected to C′, selected from the group consisting of —NR²—C(O)—NR²—, —NR²—S(O)₂—NR²—, —O—C(O)—NR²—, —NR²—C(O)—O—, —C(O)—NR²—C(O)—, —C(O)—NR²—C(R⁹R¹⁰)—, —C(R⁹R¹⁰)—NR²—C(O)—, —C(R⁹R¹⁰)—C(R⁹R¹⁰)—C(R⁹R¹⁰)—, —S(O)₂—C(R⁹R¹⁰)—C(R⁹R¹⁰)—, C(R⁹R¹⁰)—O—C(O)—, —C(R⁹R¹⁰)—O —C (R⁹R¹⁰)—, —NR²—C(O)—C(R⁹R¹⁰)—, —O—C(O)—C(R⁹R¹⁰)—, —C(R⁹R¹⁰)—C(O)—NR²—, —C(O)—NR²—C(O)—, —C(R⁹R¹⁰)—C(O)—O—, —C(O)—NR²—C(R⁹R¹⁰)—C(R⁹R¹⁰)—, —C(O)—O—C(R⁹R¹⁰)—, —C(R⁹R¹⁰)—C(R⁹R¹⁰)—C(R⁹R¹⁰)—C(R⁹R¹⁰)—, —S(O)₂NR²—C(R⁹R¹⁰)—C(R⁹¹⁰)—,—C(R⁹R¹⁰)—C(R⁹R¹⁰)—NR²—C(O)—, —C(R⁹R¹⁰)—C(R⁹R¹⁰)—O—C(O)—, —NR²—C(O)—C(R⁹R¹⁰)—C(R⁹R¹⁰)—, —NR²—S(O)₂—C(R⁹R¹⁰)—C(R⁹R¹⁰)—, —O—C(O)—C(R⁹R¹⁰)—C(R⁹R¹⁰)—, —C(R⁹R¹⁰) —C(R⁹R¹⁰)—C(O)—NR²—, —C(R⁹R¹⁰)—C(R⁹R¹⁰)—C(O)—, —C(R⁹R¹⁰)—NR²—C(O)—O—, —C(R⁹R¹⁰)—O—C(O)—NR², —C(R⁹R¹⁰)—NR²—C(O)—NR²—, —NR²—C(O)—O—C(R⁹R¹⁰)—, —NR²—C(O)—NR²—C(R⁹R¹⁰)—, —NR²—S(O)₂—NR²—C(R⁹R¹⁰)—, —O—C(O)—NR²—C(R⁹R¹⁰)—,—C(O)—N═C(R¹¹)—NR²—, —C(O)—NR²C(R¹¹)═N—, —C(R⁹R¹⁰)—NR¹²—C(R⁹R¹⁰)—, —NR ¹²—C(R⁹R¹⁰)—, —NR¹²—C(R⁹R¹⁰)—C(R⁹R¹⁰)—, —C(O)—O—C(R⁹R¹⁰)—C(R⁹R¹⁰)—, —NR²—C(R¹¹)═N—C(O)——C(R⁹R¹⁰)—C(R⁹R¹⁰)—N(R¹²)—, —C(R⁹R¹⁰)—NR¹²—, —N═C(R¹¹)—NR²—C(O)—, —C(R⁹R¹⁰)—C(R⁹R¹⁰)—NR²—S(O)₂—, —C(R⁹R¹⁰)—C(R⁹R¹⁰)—S(O)₂—NR²—C(R⁹R¹⁰)—C(R⁹R¹⁰)—C(O)—O—, —C(R⁹¹⁰)—S(O)₂—C(R⁹R¹⁰)—, —C(R⁹R¹⁰)—C(R⁹R¹⁰)—S(O)₂—, —O—C(R⁹R¹⁰)—C(R⁹R¹⁰)—, —C(R⁹R¹⁰) —C(R⁹R¹⁰)—O—, —C(R⁹R¹⁰)—C(O)—C(R⁹R¹⁰)—, —C(O)—C(R⁹R¹⁰)—C(R⁹R¹⁰)—and —C(R⁹R¹⁰)—NR²—S(O)₂—NR²—;

Q is a covalent bond or CH₂;

W is CH or N;

X is CR⁹R¹⁰, C═CH₂ or C═O;

Y is CR⁹R¹⁰, O or NR²;

Z is C═O, C═S or S(O)₂;

G¹ is hydrogen, halo, hydroxy, nitro, amino, cyano, phenyl, carboxyl, —CONH₂, —(C₁-C₄)alkyl optionally independently substituted with one or more phenyl, one or more halogens or one or more hydroxy groups, —(C₁-C₄)alkoxy optionally independently substituted with one or more phenyl, one or more halogens or one or more hydroxy groups, —(C₁-C₄)alkylthio, phenoxy, —COO(C₁-C₄)alkyl, N,N-di-(C₁-C₄)alkylamino, —(C₂-C₆)alkenyl optionally independently substituted with one or more phenyl, one or more halogens or one or more hydroxy groups, —(C₂-C₆)alkynyl optionally independently substituted with one or more phenyl, one or more halogens or one or more hydroxy groups, —(C₃-C₆)cycloalkyl optionally independently substituted with one or more (C₁-C₄)alkyl groups, one or more halogens or one or more hydroxy groups, —(C₁-C₄)alkylamino carbonyl or di—(C₁-C₄)alkylamino carbonyl;

G² and G³ are each independently selected from the group consisting of hydrogen, halo, hydroxy, —(C₁-C₄)alkyl optionally independently substituted with one to three halogens and —(C₁-C₄)alkoxy optionally independently substituted with one to three halogens;

R¹ is hydrogen, —CN, —(CH₂)_{q}N(X⁶)C(O)X⁶, —(CH₂)_{q}N(X⁶)C(O)(CH₂)ₜ-A¹, —(CH₂)_{q}N(X⁶)S(O)₂(CH₂)ₜ-A¹, —(CH₂)_{q}N(X⁶)S(O)₂X⁶, —(CH₂)_{q}N(X⁶)(CH₂)ₜ-A^{A}, —(CH₂)_{q}N(X⁶)C(O)N(X⁶)(X⁶), —(CH₂)_{q}C(O)N(X⁶)(X⁶), —(CH₂)_{q}C(O)N(X⁶)(CH₂)ₜ-A¹, —(CH₂)_{q}C(O)OX⁶, —(CH₂)_{q}C(O)O(CH₂)ₜ-A¹, —(CH₂)_{q}OX⁶, —(CH₂)_{q}OC(O)X⁶, —(CH₂)_{q}OC(O)(CH₂)ₜ-A¹, —(CH₂)_{q}OC(O)N(X⁶)(CH₂)ₜ-A¹, (CH₂)_{q}OC(O)N(X⁶)(X⁶), —(CH₂)_{q}C(O)X⁶, —(CH₂)_{q}C(O)(CH₂)ₜ-A¹, —(CH₂)_{q}N(X⁶)C(O)OX⁶, —(CH₂)_{q}N(X⁶)S(O)₂N(X⁶)(X⁶), (CH₂)_{q}S(O)ₘX⁶, (CH₂)_{q}S(O)ₘ(CH₂)ₜ-A¹, —(C₁-C₁₀)alkyl, —(CH₂)ₜ-A¹, —(CH₂)_{q}—(C₃-C₇)cycloalkyl, —(CH₂)_{q}—Y¹—(C₁-C₆)alkyl, (CH₂)_{q}—Y¹—(CH₂)ₜ-A¹ or —(CH₂)_{q}—Y¹—(CH₂)ₜ—(C₃-C₇)cycloalkyl;

where the alkyl and cycloalkyl groups in the definition of R¹ are optionally substituted with (C₁-C₄)alkyl, hydroxy, (C₁-C₄)alkoxy, carboxyl, —CONH₂, —S(O)ₘ(C₁-C₆)alkyl, —CO₂(C₁-C₄)alkyl ester, 1H-tetrazol-5-yl or 1, 2 or 3 fluoro groups;

Y¹ is O, S(O)ₘ, —C(O)NX⁶—, —CH═CH—, —C≡C—, —N(X⁶)C(O)—, —C(O)NX⁶—, —C(O)O—, —OC(O)N(X⁶)—or —OC(O)—;

q is 0, 1, 2, 3 or 4;

t is 0, 1, 2 or 3;

said (CH₂)_{q} group and (CH₂)ₜ group in the definition of R¹ are optionally independently substituted with hydroxy, (C₁-C₄)alkoxy, carboxyl, —CONH₂, —S(O)ₘ(C₁-C₆)alkyl, —CO₂(C₁-C₄)alkyl ester, 1H-tetrazol-5-yl, 1, 2 or 3 fluoro group or 1 or 2 (C₁-C₄)alkyl groups;

R^{1A} is selected from the group consisting of hydrogen, F, Cl, Br, I, (C₁-C₆)alkyl, phenyl(C₁-C₃)alkyl, pyridyl(C₁-C₃)alkyl, thiazolyl(C₁-C₃)alkyl and thienyl(C₁-C₃)alkyl, provided that R^{1A} is not F, Cl, Br or I when a heteroatom is vicinal to C″;

R², for each occurrence, is independently hydrogen, (C₁-C₈)alkyl, —(C₀-C₃)alkyl-(C₃-C₈)cycloalkyl, —(C₁-C₄)alkyl-A¹ or A¹;

where the alkyl groups and the cycloalkyl groups in the definition of R² are optionally substituted with hydroxy, —C(O)OX⁶, —C(O)N(X⁶)(X⁶), —N(X⁶)(X⁶), —S(O)ₘ(C₁-C₆)alkyl, —C(O)A¹, —C(O)(X⁶), CF₃, CN or 1, 2 or 3 independently selected halogens;

R³ and R⁴ are each independently selected from the group consisting of hydrogen, (C₁-C₈)alkyl, —CH(R⁸)-aryl, —CH(R⁸)-heteroaryl, —(C₀-C₃)alkyl(C₃-C₈)cycloalkyl, wherein the aryl or heteroaryl groups are optionally substituted by one or two R^{b} groups;

R^{b} is, for each occurrence independently, R^{c}, halo, —OR^{c}, —NHSO₂R^{c}, —N(R^{c})₂, —CN, —NO₂, —SO₂N(R^{c})₂, —SO₂R^{c}, —CF₃, —OCF₃; —OCF₂H or two R^{b} groups attached to adjacent carbon atoms taken together to form methylenedioxy;

R^{c} is, for each occurrence independently, hydrogen, —(C₁-C₈)alkyl, —(C₀-C₃)alkylaryl, —(C₀-C₃)alkylheteroaryl, (C₃-C₆)cycloalkyl; or 2 R^{b} taken together with the nitrogen atom to which they are attached to form a 5- or 6- membered ring optionally containing an additional heteroatom selected from O, S or NR³;

R⁶ and R⁷ are each independently selected from hydrogen, (C₁-C₆)alkyl, —(C₀-C₃)alkylaryl, —(C₀-C₃)alkylheteroaryl, —(C₀-C₃)alkyl(C₃-C₈)cycloalkyl;

or R⁶ and R⁷ together with the nitrogen atom to which they are attached form a 5- or 6-membered ring optionally containing an additional heteroatom selected from O, S, NR³;

D is —(C₀-C₆)alkyl-amino—C(═NR⁷)—NR¹⁵R¹6, —(C₀-C₆)alkylaminopyridyl, —(C₀-C₆)alkylaminoimidazolyl, —(C₀-C₆)alkylaminothiazolyl, —(C₀-C₆)alkylaminopyrimidinyl, (C₀-C₆)alkylaminopiperazinyl-R¹⁵, —(C₀-C₆)alkylmorpholinyl, wherein R¹⁵ and R¹⁶ are independently hydrogen, —(C₁-C₆)alkyl, —(C₀-C₃)alkylaryl, —(C₀-C₃)alkylheteroaryl, —(C_{-C}₃)alkyl(C₃-C₈)cycloalkyl, wherein the alkyl and aryl groups are optionally substituted with one or two R^{b} groups; or D is a group of the formula

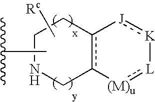

wherein the dashed lines represent optional double bonds;

u is 0 or 1;

x and y are each independently 0, 1 or 2;

J, K, L and M are each independently selected from C(R^{b})ᵣ, N, S or O wherein R^{b} and R^{c} are as defined above and r is 1 or 2;

X⁴ is hydrogen or (C₁-C₆)alkyl or X⁴ is taken together with R⁴ and the nitrogen atom to which X⁴ is attached and the carbon atom to which R⁴ is attached and form a five to seven membered ring;

R⁸ is hydrogen, —(C₁-C₈)alkyl, —(C₀-C₃)alkylaryl, —(C₀-C₃)alkylheteroaryl, —(C₃-C₆)cycloalkyl; or 2 R^{b} taken together with the nitrogen atom to which they are attached to form a 5- or 6- membered ring optionally containing an additional heteroaryl selected from O, S or NR³;

R⁹ and R¹⁰, for each occurrence independently, are each independently selected from the group consisting of hydrogen, fluoro, hydroxy and (C₁-C₅)alkyl optionally independently substituted with 1-5 halogens;

R¹¹ is selected from the group consisting of (C₁-C₅)alkyl and phenyl optionally substituted with 1-3 substituents each independently selected from the group consisting of (C₁-C₅)alkyl, halo and (C₁-C₅)alkoxy;

R¹² is selected from the group consisting of (C₁-C₅)alkylsulfonyl, (C₁-C₅)alkanoyl and (C₁-C₅)alkyl where the alkyl portion is optionally independently substituted by 1-5 halogens;

A¹ for each occurrence is independently selected from the group consisting of (C₅-C₇)cycloalkenyl, phenyl, a partially saturated, fully saturated or fully unsaturated 4- to 8-membered ring optionally having 1 to 4 heteroatoms independently selected from the group consisting of oxygen, sulfur and nitrogen and a bicyclic ring system consisting of a partially saturated, fully unsaturated or fully saturated 5- or 6-membered ring, optionally having 1 to 4 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;

A¹ for each occurrence is independently optionally substituted, on one or optionally both rings if A¹ is a bicyclic ring system, with up to three substituents, each substituent independently selected from the group consisting of F, Cl, Br, I, —OCF₃, —OCF₂H, —CF₃, —CH₃, —OCH₃, —OX⁶, —C(O)N(X⁶)(X⁶), —C(O)OX⁶, oxo, (C₁-C₆)alkyl, nitro, cyano, benzyl, —S(O)ₘ(C₁-C₆)alkyl, 1H-tetrazol-5-yl, phenyl, phenoxy, phenylalkyloxy, halophenyl, methylenedioxy, —N(X⁶)(X⁶), —N(X⁶)C(O)(X⁶), —S(O)₂N(X⁶)(X⁶), —N(X⁶)S(O)₂-phenyl, —N(X⁶)S(O)₂X⁶, —CONX¹¹X¹², —S(O)₂NX¹¹X¹², —NX⁶S(O)₂X¹², —NX⁶CONX¹¹X¹², —NX⁶S(O)₂NX¹¹X¹², —NX⁶C(O)X¹², imidazolyl, thiazolyl and tetrazolyl, provided that if A¹ is optionally substituted with methylenedioxy then it can only be substituted with one methylenedioxy;

where X¹¹, for each occurrence, is independently hydrogen or optionally substituted (C₁-C₆)alkyl;

the optionally substituted (C₁-C₆)alkyl defined for X¹¹ is optionally independently substituted with phenyl, phenoxy, (C₁-C₆)alkoxycarbonyl, —S(O)ₘ(C₁-C₆)alkyl, 1 to 5 halogens, 1 to 3 hydroxy groups, 1 to 3 (C₁-C₁₀)alkanoyloxy groups or 1 to 3 (C₁-C₆)alkoxy groups;

X¹², for each occurrence, is independently hydrogen, (C₁-C₆)alkyl, phenyl, thiazolyl, imidazolyl, furyl or thienyl, provided that when X¹² is not hydrogen, the X¹² group is optionally substituted with one to three substituents independently selected from the group consisting of Cl, F, CH₃, OCH₃, OCF₃ and CF₃;

or X¹¹ and X¹² are taken together to form —(CH₂)_{g}-L¹—(CH₂)_{g}—;

L¹ is C(X²)(X²), O, S(O)ₘ or N(X²);

g for each occurrence is independently 1, 2 or 3;

X² for each occurrence is independently hydrogen, optionally substituted (C₁-C₆)alkyl or optionally substituted (C₃-C₇)cycloalkyl, where the optionally substituted (C₁-C₆)alkyl and optionally substituted (C₃-C₇)cycloalkyl in the definition of X² are optionally independently substituted with —S(O)ₘ(C₁-C₆)alkyl, —C(O)OX³, 1 to 5 halogens or 1-3 OX³ groups;

X³ for each occurrence is independently hydrogen or (C₁-C₆)alkyl;

X⁶ for each occurrence is independently hydrogen, optionally substituted (C₁-C₆)alkyl, (C₂-C₆)halogenated alkyl, optionally substituted (C₃-C₇)cycloalkyl, (C₃-C₇)—halogenated cycloalkyl, where optionally substituted (C₁-C₆)alkyl and optionally substituted (C₃-C₇)cycloalkyl in the definition of X⁶ is optionally independently mono-or di-substituted with (C₁-C₄)alkyl, hydroxy, (C₁-C₄)alkoxy, carboxyl, CONH₂, —S(O)ₘ(C₁-C₆)alkyl, carboxylate (C₁-C₄)alkyl ester or 1H-tetrazol-5-yl; or when there are two X⁶ groups on one atom and both X⁶ are independently (C₁-C₆)alkyl, the two (C₁-C₆)alkyl groups may be optionally joined and, together with the atom to which the two X⁶ groups are attached, form a 4- to 9- membered ring optionally having oxygen, sulfur or NX⁷ as a ring member;

X⁷, for each occurrence independently, is hydrogen or (C₁-C₆)alkyl optionally substituted with hydroxy;

m for each occurrence is independently 0, 1 or 2;

with the proviso that: X⁶ and X¹² cannot be hydrogen when attached to C(O) or S(O)₂ in the form C(O)X⁶, C(O)X¹², S(O)₂X⁶ or S(O)₂X¹².

The present invention further relates to a compound of formula I wherein D is

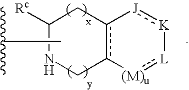

The present invention further relates to a compound of formula I wherein x is 1, y is 1 and u is 1.

The present invention further relates to a compound of formula I wherein J, K, L and M are each NR^{b} or C(R^{b})ᵣ where r=1 or 2, R⁴ is —CH₂-aryl in which aryl is optionally substituted by R^{b}

The present invention further relates to a compound of formula I wherein HET is

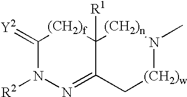

The present invention further relates to a compound of formula I wherein Y² is oxygen, f is 0, n is 1 or 2; and w is 0 or 1.

The present invention further relates to a compound of formula I wherein R² is (C₁-C₆)alkyl optionally substituted by halo, R³ is hydrogen, n is 1, w is 1, and R¹ is aryl(C₁-C₆)alkyl, (C₁-C₆)alkyl or heteroaryl(C₁-C₆)alkyl wherein aryl and heteroaryl are optionally substituted with one or two groups from the following list: halo, —OR^{c}, —NHSO₂R^{c}, —N(R^{c})₂, —CN, —NO₂, —SO₂N(R^{c})₂, —SO₂R^{c}, —CF₃, —OCF₃; —OCF₂H.

The present invention further relates to a compound of formula I wherein J, K, L and M are each N or CR^{b} and the dashed lines represent double bonds, R¹ is benzyl optionally substituted by halo, -R^{c}, —OR^{c}, —CF₃, —OCF₃, —OCF₂H, R^{c}, hydrogen, —(C₁-C₆)alkyl, —(C₀-C₃)alkylaryl, —(C₀C₃)alkylheteroaryl or —(C₃-C₆)cycloalkyl.

Specific preferred compounds of formula I include those wherein said compound is selected from the group consisting of:

1,2,3,4-Tetrahydro-isoquinoline-(S)3-carboxylic acid [2-((R)3a-benzyl-2-methyl-3-oxo-2,3,3a,4,6,7-hexahydro-pyrazolo[4,3-c]pyridin-5-yl) —(R)1 -(4-chloro-benzyl)-2-oxo-ethyl]-amide;

1,2,3,4-Tetrahydro-isoquinoline-(R)3-carboxylic acid [2-((R)3a-benzyl-2-methyl-3-oxo-2,3,3a,4,6,7-hexahydro-pyrazolo[4,3-c]pyridin-5-yl) —(R)1 -(4-chloro-benzyl)-2-oxo-ethyl]-amide;

1,2,3,4-Tetrahydro-isoquinoline-(R)3-carboxylic acid [2-[3a-benzyl-3-oxo-2-(2,2,2-trifluoro-ethyl)-2,3,3a,4,6,7-hexahydro-pyrazolo[4,3-c]pyridin-5-yl] —(R)1-(4-chloro-benzyl)-2-oxo-ethyl]-amide;

1,2,3,4-Tetrahydro-isoquinoline-(R)3-carboxylic acid {(R)1-(4-chloro-benzyl)-2-[2-ethyl-(S)3a-(4-fluoro-benzyl)-3-oxo-2,3,3a,4,6,7-hexahydro-pyrazolo[4,3-c]pyridin-5-yl]-2-oxo-ethyl}-amide;

1,2,3,4-Tetrahydro-isoquinoline-(S)3-carboxylic acid {(R)1-(4-chloro-benzyl)-2-[2-ethyl-(S)3a-(4-fluoro-benzyl)-3-oxo-2,3,3a,4,6,7-hexahydro-pyrazolo[4,3-c]pyridin-5-yl]-2-oxo-ethyl}-amide;

1,2,3,4-Tetrahydro-isoquinoline-(S)3-carboxylic acid {(R)1-(4-chloro-benzyl)-2-[(S)3a-(4-chloro-benzyl)-2-ethyl-3-oxo-2,3,3a,4,6,7-hexahydro-pyrazolo[4,3-c]pyridin-5-yl]-2-oxo-ethyl}-amide;

1,2,3,4-Tetrahydro-isoquinoline-(R)3-carboxylic acid {(R)1-(4-chloro-benzyl)-2-[(S)3a-(4-chloro-benzyl)-2-ethyl-3-oxo-2,3,3a,4,6,7-hexahydro-pyrazolo[4,3-c]pyridin-5-yl]-2-oxo-ethyl}-amide;

1,2,3,4-Tetrahydro-isoquinoline-(R)3-carboxylic acid [2-((S)3a-benzyl-2-methyl-3-oxo-2,3,3a,4,6,7-hexahydro-pyrazolo[4,3-c]pyridin-5-yl)-(R)1 -(4-chloro-benzyl)-2-oxo-ethyl]-amide;

1,2,3,4-Tetrahydro-isoquinoline-(R)3-carboxylic acid {(R)1-(4-chloro-benzyl)-2-[(R)3a-(3-fluoro-benzyl)-3-oxo-2,3,3a,4,6,7-hexahydro-pyrazolo[4,3-c]pyridin-5-yl]-2-oxo-ethyl}-amide;

1,2,3,4-Tetrahydro-isoquinoline-(S)3-carboxylic acid [2-[3a-benzyl-3-oxo-2-(2,2,2-trifluoro-ethyl)-2,3,3a,4,6,7-hexahydro-pyrazolo[4,3-c]pyridin-5-yl]—(R)1 -(4-chloro-benzyl)-2-oxo-ethyl]-amide; and

1,2,3,4-Tetrahydro-isoquinoline-(R)3-carboxylic acid [(R)1-(4-chloro-benzyl)-2-oxo-2-(3-oxo-3a-pyridin-2-ylmethyl-2,3,3a,4,6,7-hexahydro-pyrazolo[4,3-c]pyridin-5-yl)-ethyl]-amide.

The present invention further relates to a compound of formula I wherein J, K, L and M are each NR^{b} or C(R^{b})₂ and the dashed lines represent single bonds, wherein R^{b} is hydrogen, halo, R^{c}, —OR^{c}, —CF₃, —OCF₃, —OCF₂H, R^{c} is hydrogen, (C₁-C₈)alkyl, (C₀-C₃)alkylaryl, (C₀-C₃)alkylheteroaryl or —(C₃-C₆)cycloalkyl.

The present invention further relates to a compound of formula I wherein HET is

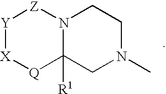

The present invention further relates to a compound of formula I wherein Q is a covalent bond; X and Z are each C═O; and Y is NR².

The present invention further relates to a compound of formula I wherein R² is (C₁-C₆)alkyl optionally substituted by halo, and R¹ is aryl(C₁-C₆)alkyl, (C₁-C₆)alkyl or heteroaryl (C₁-C₆)alkyl wherein aryl and heteroaryl are optionally substituted with one or two groups from the following list: halo, OR^{c}, —NHSO₂R^{c}, N(R^{c})₂, CN, NO₂, SO₂N(R^{c})₂, —SO₂R^{c}, —CF₃, —OCF₃, —OCF₂H.

The present invention further relates to a compound of formula I wherein J, K, L and M are each N or CR^{b} and the dashed lines represent double bonds, R¹ is benzyl optionally substituted by halo, —R^{c}, —OR^{c}, —OCF₃, —OCF₂H, and R^{c} is hydrogen, —(C₁-C₈)alkyl, —(C₀-C₃)akylaryl, —(C₀-C₃)alkylheteroaryl or —(C₃-C₆)cycloalkyl.

Specific preferred compounds of formula I include those wherein said compound is selected from the group consisting of:

1,2,3,4-Tetrahydro-isoquinoline-(S)3-carboxylic acid {(R)1-(4-chloro-benzyl)-2-[1,3-dioxo-(S)8a-pyridin-2-ylmethyl-2-(2,2,2-trifluoro-ethyl)-hexahydro-imidazo[1,5-a]pyrazin-7-yl]-2-oxo-ethyl}-amide;

1,2,3,4-Tetrahydro-isoquinoline-(R)3-carboxylic acid {(R)1-(4-chloro-benzyl)-2-[(R)8a-(4-fluoro-benzyl)-2-methyl-1,3-dioxo-hexahydro-imidazo[1,5-a]pyrazin-7-yl]-2-oxo-ethyl}-amide;

1,2,3,4-Tetrahydro-isoquinoline-(S)3-carboxylic acid {(R)1-(4-chloro-benzyl)-2-[1,3-dioxo-(S)8a-pyridin-3-ylmethyl-2-(2,2,2-trifluoro-ethyl)-hexahydro-imidazo[1,5-a]pyrazin-7-yl]-2-oxo-ethyl}-amide;

1,2,3,4-Tetrahydro-isoquinoline-(S)3-carboxylic acid {(R)1-(4-chloro-benzyl)-2-[8a-(4-fluoro-benzyl)-3-oxo-tetrahydro-oxazolo[3,4-a]pyrazin-7-yl]-2-oxo-ethyl}-amide;

1,2,3,4-Tetrahydro-isoquinoline-(S)3-carboxylic acid {(R)1-(4-chloro-benzyl)-2-[8a-(4-fluoro-benzyl)-2-methyl-1,3-dioxo-hexahydro-imidazo[1,5-a]pyrazin-7-yl]-2-oxo-ethyl}-amide; and

1,2,3,4-Tetrahydro-isoquinoline-(S)3-carboxylic acid {(R)1-(4-chloro-benzyl)-2-[8a-(4-fluoro-benzyl)-2-methyl-1,3-dioxo-hexahydro-imidazo[1,5-a]pyrazin-7-yl]-2-oxo-ethyl}-amide.

The present invention further relates to a compound of formula I wherein J, K, L and M are each NR^{b} or C(R^{b})₂ and the dashed lines represent single bonds, R^{b} is hydrogen, halo, R^{c}, OR^{c}, —CF₃, —OCF₃, —OCF₂H, R^{c} is hydrogen, —(C₁-C₈)alkyl, -(C₀-C₃)alkylaryl, —(C₀-C₃)alkylheteroaryl or —(C₃-C₆)cycloalkyl.

The present invention relates to a method for the treatment or prevention of disorders, diseases or conditions responsive to the activation of melanocortin receptor which comprises administering to a mammal in need of such treatment or prevention an effective amount of a compound of formula 1.

The present invention relates to a method for the treatment or prevention of obesity which comprises administering to a mammal in need of such treatment or prevention an effective amount of a compound of formula 1.

The present invention relates to a method for the treatment or prevention of diabetes mellitus which comprises administering to a mammal in need of such treatment or prevention an effective amount of formula 1.

The present invention relates to a method for the treatment or prevention of male or female sexual dysfunction which comprises administering to a mammal in need of such treatment or prevention an effective amount of a compound of formula 1.

The present invention relates to a method for the treatment or prevention of erectile dysfunction which comprises administering to a mammal in need of such treatment or prevention an effective amount of a compound of formula 1.

The present invention relates to a method for modulating appetite and metabolic rates of mammals which comprises administering to a mammal in need of such treatment or prevention an effective amount of a compound of formula 1.

The present invention relates to a method for treating or preventing disorders that cause reduction in appetite, feeding behavior and/or body weight in a mammal which comprises administering to a mammal in need of such treatment or prevention an effective amount of a compound of formula 1.

The present invention relates to a method for acutely stimulating the appetite of companion animals for the treatment of hepatic lipidosis, cachexia and other pathologies resulting in/from inappropriate food intake and weight loss, which comprises administering to a mammal in need of such treatment or prevention an effective amount of a compound of formula 1.

The present invention relates to a method for acutely stimulating the appetite of livestock for the treatment of ketosis, postpartum anestrus, and other metabolic and reproductive pathologies resulting in/from inappropriate food intake and weight loss which comprises administering to a mammal in need of such treatment or prevention an effective amount of a compound of formula 1.

The present invention relates to a method that will enhance growth and survivability of neonates in livestock which comprises administering to a mammal in need of such treatment or prevention an effective amount of a compound of formula 1.

The present invention relates to a pharmaceutical composition, which comprises a compound of formula 1, a pharmaceutically acceptable carrier.

The present invention relates to a pharmaceutical composition of the compound of formula I further comprising a second active ingredient selected from an insulin sensitizer, insulin mimetic, sulfonylurea, α-glucosidase inhibitor, HMG-CoA reductase inhibitor, sequestrant cholesterol lowering agent, β3 adrenergic receptor agonists, neuropeptide Y antagonist, phosphodiester V inhibitor, and α-2 adrenergic receptor antagonist.

### DETAILED DESCRIPTION OF THE INVENTION

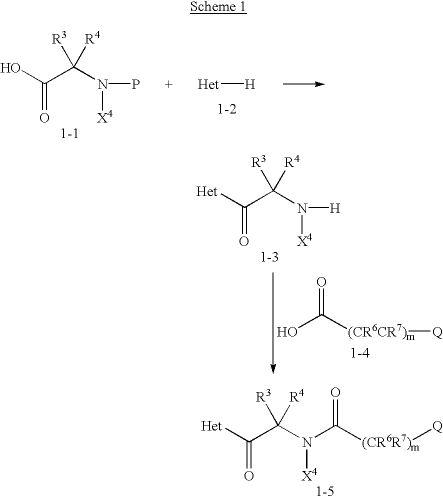

As illustrated in Scheme 1, compound 1-3 can be prepared by coupling of a protected amino acid of formula 1-1 with a heterocyclic amine of formula 1-2, as defined in claim 1, with a coupling agent such as n-propylphosphonic anhydride (PPAA), with or without a base, such as triethylamine, in a solvent, such as ethyl acetate, from −20° C. to room temperature followed by deprotection of a suitable protecting group (P) that are well known in the art (e.g. Green, T. W., Wells, P. G. M., “Protecting Groups in Organic Synthesis,” 1991, John Wiley & Sons, Inc.). An example of a suitable protecting group is the t-butyl carbamate group (BOC). The BOC group can be removed by the treatment of the protected intermediate with an acid, for example, hydrochloric acid, in a solvent, for example, dioxane, ethyl ether, and/or ethyl acetate, from 000 to room temperature. Compound 1-5 can be prepared by coupling an acid of formula 1-4 (prepared as described in WO 99/64002, which is incorporated by reference in its entirety) with an amine of formula 1-3 with a coupling agent, such as benzotriazol-1 -yloxy-tris(dimethylamino) phosphonium hexafluorophosphate (BOP) or PPAA, with or without a base, such as triethylamine or diisopropylethylamine, in a solvent such as ethyl acetate or dichloromethane, from −20° C. to room temperature.

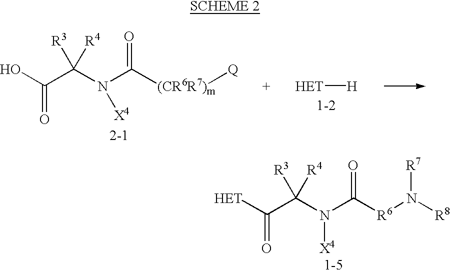

Alternatively, compounds 1-5 can be prepared as illustrated in Scheme 2. Compounds 1-5 can be prepared by coupling acid 2-1 with a heterocyclic amine of formula 1-2, as defined in claim 1, with a coupling agent such as PPAA, with or without a base, such as triethylamine or diisoprylethylamine, in a solvent such as ethyl acetate, from −20° C. to room temperature. Any suitable protecting group on Q can then be removed under conditions well known in the art (e.g. Green, T. W., Wells, P. G. M., “Protecting Groups in Organic Synthesis,” 1991, John Wiley & Sons, Inc.). An example of a suitable protecting group is the BOC group. The BOC group can be removed by treatment of the protected intermediate with an acid, for example hydrochloric acid, in a solvent, for example, dioxane ethyl ether, and/or ethylacetate, from 0° C. to room temperature.

[chemical structure]

As illustrated in Scheme 3, intermediates of formula 3-2 can be prepared by treating an acid of formula 3-1 with hydroxysuccinimide in the presence of a coupling agent such as EDC in an inert solvent such as methylene chloride. Treating 3-2 with an amino acid of formula 3-3 in a solvent such as DMF in the presence of a base such as diisopropylethylamine produces compounds of formula 2-1.

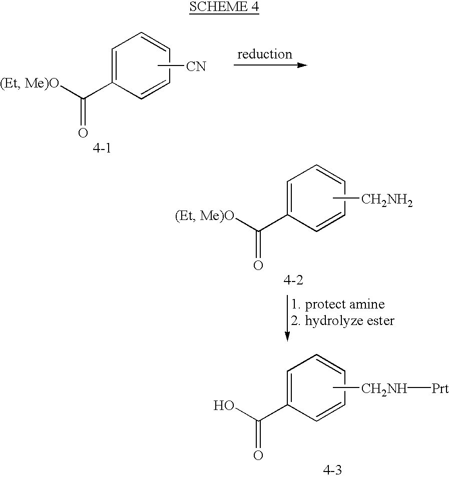

As illustrated in Scheme 4, benzoic acid esters of formula 4-1 are reduced, e.g., with Raney nickel in ethanol in the presence of ammonia to provide the corresponding benzylamine derivative 4-2. The amino group is protected according to methods well known to those skilled in the art, e.g., as a BOC or CBZ derivative and the ester group is hydrolyzed to afford the protected amino acids of formula 4-3.

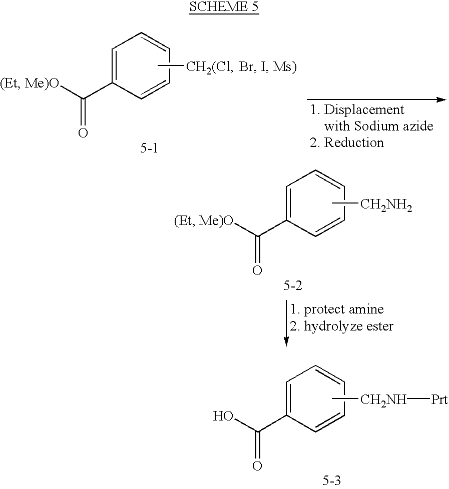

As illustrated in Scheme 5, compounds of the formula 5-3 can be prepared from the corresponding benzyl compounds (e.g., benzyl halides, benzyl mesylates) of formula 5-1. The leaving group (e.g., halide, mesylate) is displaced with sodium azide, usually in a polar aprotic solvent such as DMF or DMSO to afford the corresponding azide which is reduced, e.g., with triphenylphosphine in THF-water, to afford the amine derivative, which is converted to acids of formula 5-3.

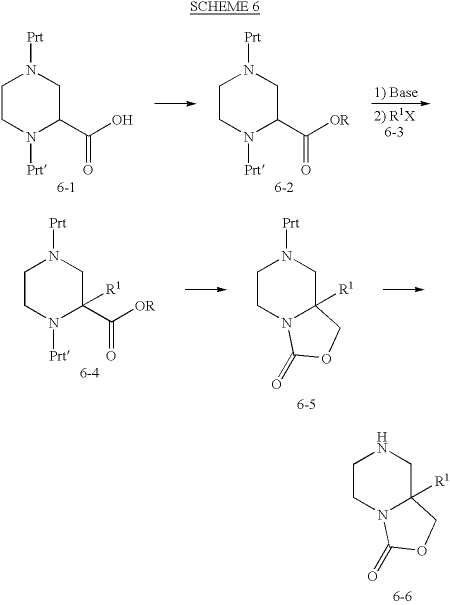

Intermediate esters of formula 6-2, where Prt and Prt′ are protecting groups, preferably Prt′ is a carbamate protecting group such as CBZ, can be prepared by treating an acid of formula 6-1 with a base such as potassium carbonate followed by an alkyl halide such as iodomethane in a suitable solvent such as DMF. Alternatively, an ester of formula 6-2 can be prepared by reacting an acid of formula 6-1 with diazomethane. For the preparation of compound 6-2 see Bigge, C. F. et al., Tet. Lett., 1989, 30, 5193-5196. Intermediate 6-4 is generated by alkylating ester 6-2 with a reagent such as an alkyl halide, tosylate or mesylate with a base such as NaHMDS in a suitable solvent system such as DMF/THF at a temperature of about −78° C.

Intermediate carbamates of formula 6-5 can be prepared by reacting an intermediate of formula 6-4 with a hydride such as sodium borohydride or superhydride. Transformation of intermediate 6-5 to 6-6 can be achieved by removal of the protecting group Prt as described above.

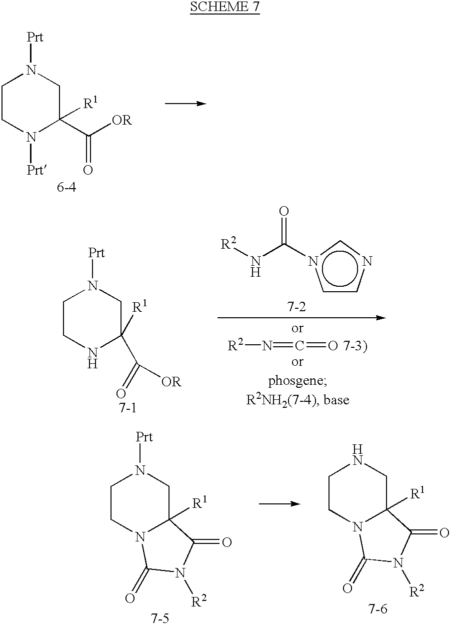

Transformation of intermediate 6-4 to 7-1 can be achieved by removal of the protecting group Prt′ as described above. Intermediate ureas of formula 7-5 can be prepared by reacting an intermediate of formula 7-1 with either an acyl imidizolide of formula 7-2, an isocyanate of formula 7-3, or phosgene (or other phosgene equivalent) followed by an amine of formula 7-4 in the presence of a suitable base such as triethylamine. When R¹ is —CH₂-pyridyl it is preferred to use an isocyanate or acyl imidizolide. Transformation of 7-5 to 7-6 can be achieved by removal of the protecting group Prt as described above.

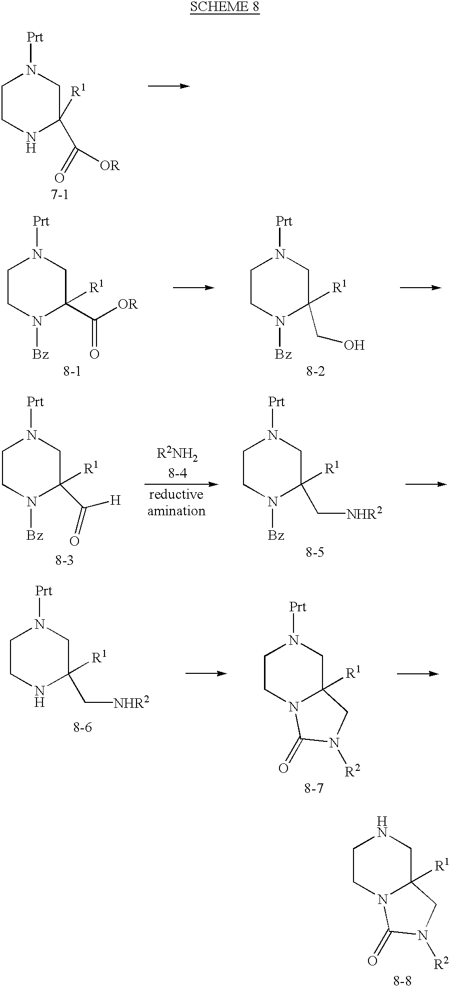

An intermediate benzylamine of formula 8-1 can be prepared by treating an amine of formula 7-1 with a base such as diisopropylethylamine followed by a benzyl halide such as benzyl bromide in a suitable solvent such as acetonitrile. Alternatively, 8-1 can be prepared by treating 7-1 with benzaldehyde and a suitable reducing agent such as NaCNBH₃ or Na(OAc)₃BH in a suitable solvent such as methanol or dichloromethane. An alcohol of the formula 8-2 can be prepared by reducing an intermediate of the formula 8-1 with a reducing agent such as superhydride in a suitable solvent such as THF. An alcohol of the formula 8-2 can be oxidized to an aldehyde of the formula 8-3 with an oxidizing agent such as oxalyl chloride/DMSO in a suitable solvent such as dichloromethane at a temperature of about −78° C., with the later addition of a base such as triethylamine to neutralize the reaction mixture (Swern-type oxidation, see Mancuso, A. J., Swern, D., Synthesis, 1981, pp. 165-185). Compounds of formula 8-5 can be prepared by treating an aldehyde of formula 8-3 with an amine of formula 8-4 in the presence of a suitable reducing agent which include alkali metal borohydrides and cyanoborohydrides. The preferred reducing agent is sodium cyanoborohydride. Sodium borohydride and sodium triacetoxyborohydride may also be used. For a general review of reductive aminations see R. F. Borch, Aldrichimica Acta, 8, 3-10 (1975). Removal of the benzyl group to give 8-6 can be accomplished by a number of reductive methods including hydrogenation in the presence of platinum or palladium catalyst in a protic solvent such as methanol. Cyclization of a diamine of formula 8-6 with CDl or other phosgene equivalents generates a compound of formula 8-7. Removal of the protecting group, as described above, transforms 8-7 into 8-8.

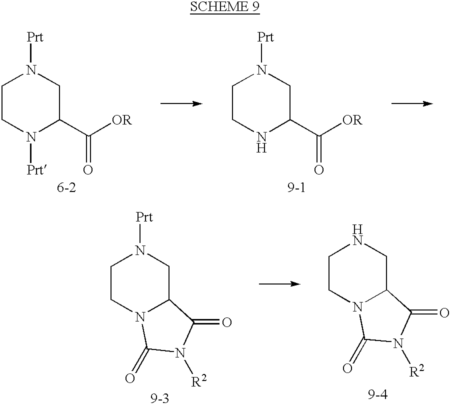

As illustrated in Scheme 9, an intermediate hydantoin of formula 9-4 can be prepared in three steps. An ester of formula 9-1, prepared by cleavage of Prt′ from 6-2, can be acylated with an acyl imidizolide of formula 7-2, an isocyanate of formula 7-3, or phosgene (or other phosgene equivalent) followed by an amine of formula 7-4 in the presence of a suitable base such as triethylamine. Transformation of 9-3 to 9-4 can be accomplished by removal of the protecting group Prt as described above.

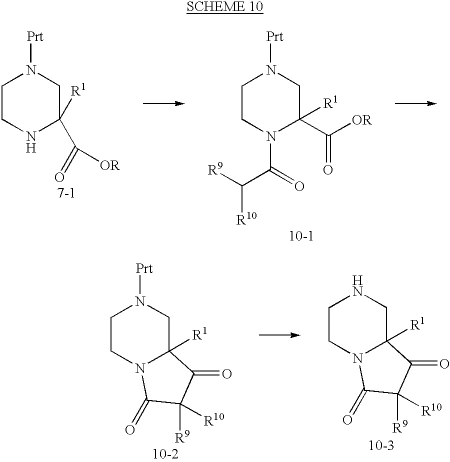

Intermediates of formula 10-1 can be prepared by treating a compound of formula 7-1 with an acyl chloride or other activated carboxylic acid derivative and a suitable base, such as TEA or N,N-diisopropylethylamine. Cyclization of a compound of formula 10-1 occurs upon treating 10-1 with a strong base such as LHMDS at a suitable temperature, about −78° C. to 40° C., to produce an intermediate of formula 10-2. When R⁹ and/or R¹⁰ is H, 10-2 may be alkylated with a reagent such as methyl iodide in the presence of a base like NaH to give 10-2 where R⁹ and R¹⁰ are not H. Removal of the protecting group, as described above, transforms 10-2 to 10-3.

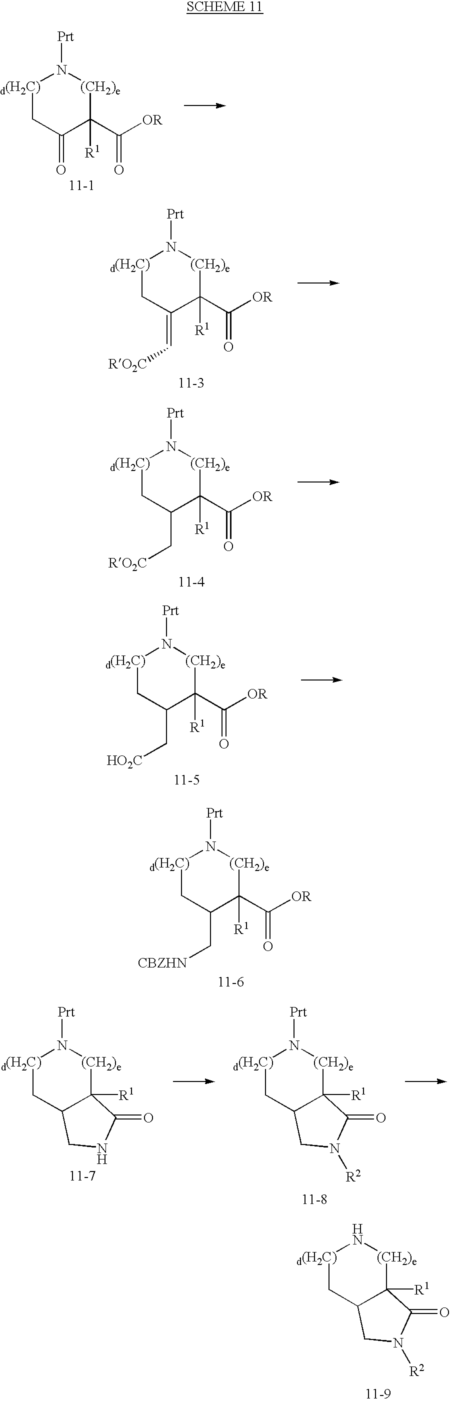

Intermediate α,β-unsaturated esters of formula 11-3 (R is an alkyl group) can be prepared by olefinating 11-1 with a reagent such as the anion generated upon treating trimethylphosphonoacetate with a strong base such as potassium tert-butoxide in a suitable solvent such as THF. Catalytic hydrogenation, such as with Pd on carbon in the presence of hydrogen, preferably at 1-4 atmospheres, in a suitable solvent, such as ethyl acetate or methanol, reduces the double bond of 11-3 to produce 11-4. Selective hydrolysis of the less hindered ester group in 11-4 can be performed with a base such as an alkali metal hydroxide in an appropriate solvent, such as a mixture of water, methanol, and/or dioxane. A carboxylic acid of formula 11-5, thus produced can be transformed to 11-6 by converting 11-5 to an acyl azide, such as with DPPA and TEA in benzene, followed by rearrangement to an isocyanate by heating to reflux in a solvent such as benzene, which is then reacted with benzyl alcohol to form 11-6. A lactam of formula 11-7 can be prepared by removal of the CBZ protecting group from the amine in 11-6, followed by cyclization of the amine with the adjacent ester group. Deprotection of this material provides 11-9, R²=H. Alternatively, amide 11-7 can be alkylated by deprotonation with a strong base such as sodium hydride, LHMDS, or KHMDS in a suitable solvent such as DMF or THF followed by treatment with an alkylating agent such as an alkyl halide, mesylate or tosylate. The product, 11-8, may then be deprotected, as described above, to provide 11-9. One skilled in the art will recognize that substitution next to the lactam nitrogen could have been introduced by alkylating ester 11-4 or by olefinating 11-1 to give a tetra-substituted olefin analogous to 11-3.

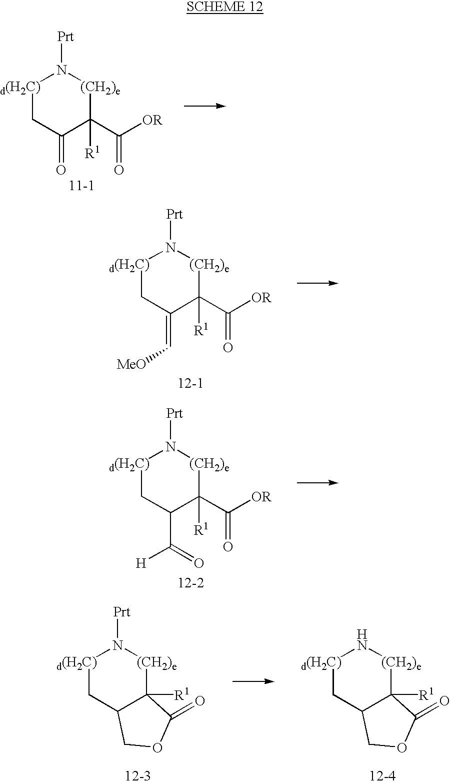

Intermediate enol ethers of formula 12-1 can be prepared by treating 11-1 (R is an alkyl group) with a reagent, such as methoxymethyl triphenylphosphonium chloride and a strong base, such as potassium tert-butoxide, in a suitable solvent such as THF. Hydrolysis of an enol ether of formula 12-1 under acidic conditions produces aldehyde 12-2. Reduction of the aldehyde group to an alcohol, for example with sodium borohydride in methanol, followed by cyclization converts 12-2 to a lactone of formula 12-3. Deprotection of the nitrogen, as described above, affords 12-4. One skilled in the art will recognize that an R^{1A} substituent could have been introduced by alkylating aldehyde 12-2. In addition, substitution next to the lactone oxygen (R⁹/R¹⁰) could be introduced by olefinating 11-1 to give a tetra-substituted olefin and by treating the latter ketone or aldehyde (12-2) with an alkyl metal such as a Grignard reagent.

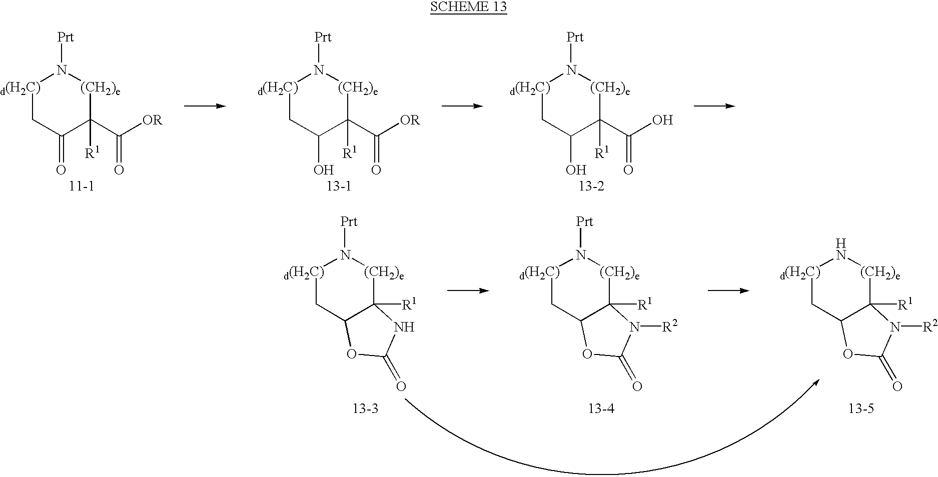

Reduction of the ketone in 11-1 (R is an alkyl group) to an alcohol with a suitable reducing reagent, such as with sodium borohydride in methanol, converts 11-1 to 13-1. Hydrolysis of the ester group in 13-1 according to the method discussed in Scheme 11 produces acid 13-2. Transformation of 13-2 to 13-3 can be achieved by converting 13-2 to acyl azides, for instance with DPPA and TEA in a solvent such as benzene, followed by rearrangement to isocyanates, which then react intramolecularly with the adjacent alcohol to form carbamate 13-3. Deprotection of 13-3 as described above provides 13-5 where R² is H. Alternatively, carbamate 13-3 can be alkylated by deprotonation with a strong base such as sodium hydride, LHMDS, or KHMDS in a suitable solvent such as DMF or THF followed by treatment with an alkylating agent such as an alkyl halide (R²-halide), mesylate or tosylate. Removal of the protecting group, as described above, transforms 13-4 to 13-5. One skilled in the art will recognize that an R^{1A} substituent could have been introduced by treating ketone 11-1 with an alkyl metal reagent, such as methyl magnesium bromide, at a suitable temperature for a Grignard reaction.

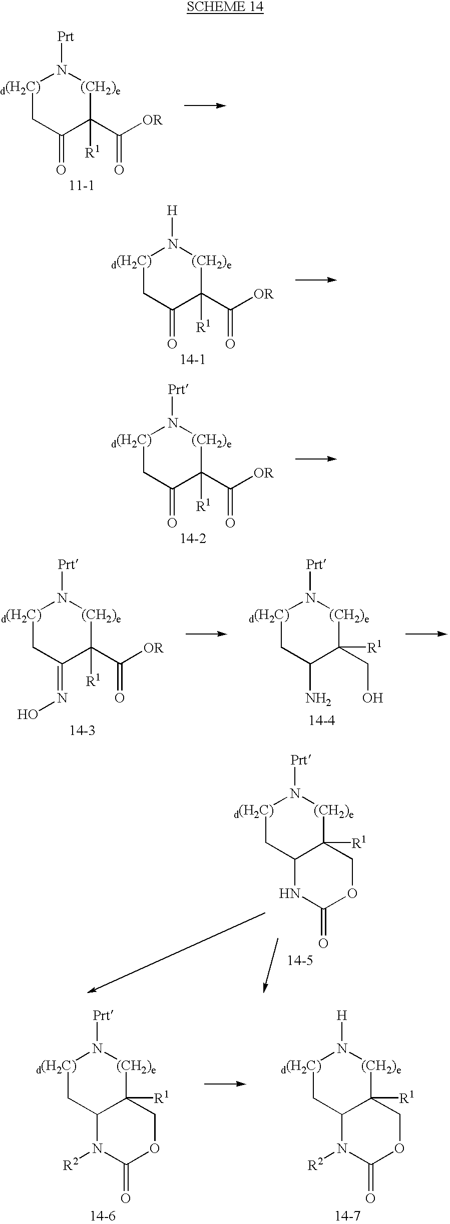

Removal of the carbamate protecting group, Prt, from 11-1 (R is an alkyl group) produces 14-1. Reprotection, such as with a benzyl group gives 14-2. Treating 14-2 with hydroxylamine yields an oxime of formula 14-3. The oxime and ester groups in 14-3 can be reduced to an amine and alcohol, respectively, to form 14-4 with a suitable reducing reagent, such as with LAH in THF. Transformation of 14-4 to a carbamate of formula 14-5 can be achieved by reaction of 14-4 with CDl or another phosgene equivalent in the presence of a base like TEA and solvent such as DME. Deprotection of 14-5 produces 14-7 where R² is H. Alternatively, alkylation of the carbamate as described above (Scheme 13) affords 14-6, which can be deprotected, as described above, to give 14-7.

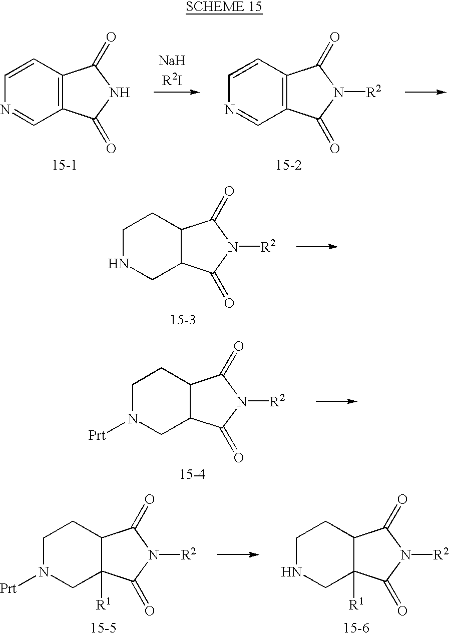

Treating 15-1 with a strong base such as sodium hydride in a suitable solvent such as DMF, followed by treatment with an alkylating agent, such as an alkyl halide, mesylate or tosylate, produces an N-substituted imide of formula 15-2. Reduction of the pyridine ring by catalytic hydrogenation, such as with Pd on carbon in an ethanolic HCl solution converts 15-2 to 15-3. Protection of the nitrogen, such as with a benzyl group, gives 15-4. A compound of the formula 15-5 can be generated upon deprotonation of 15-4 with a suitable strong base such as LHMDS in a solvent such as THF at a temperature of about −78 ° C., followed by alkylation with an electrophile such as an alkyl halide such as benzyl bromide. Cleavage of the protecting group, as described above, then gives 15-6.

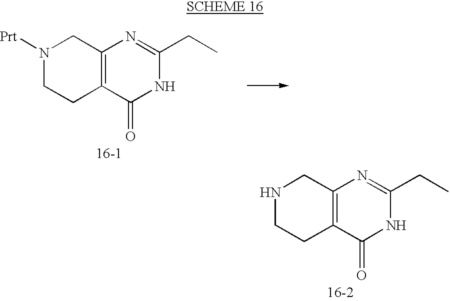

Deprotection of 16-1 as described above produces 16-2.

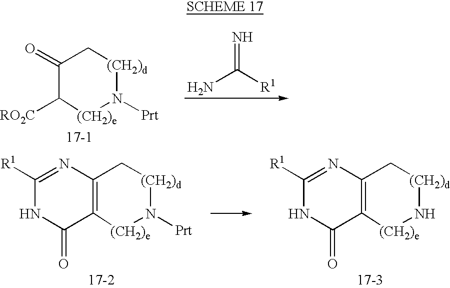

Condensation of 17-1 (R is an alkyl group) with an amidine in a solvent such as ethanol at an elevated temperature, preferably refluxing solvent, produces a heterocyclic intermediate of formula 17-2. Deprotection of 17-2, as described above, gives an intermediate of formula 17-3.

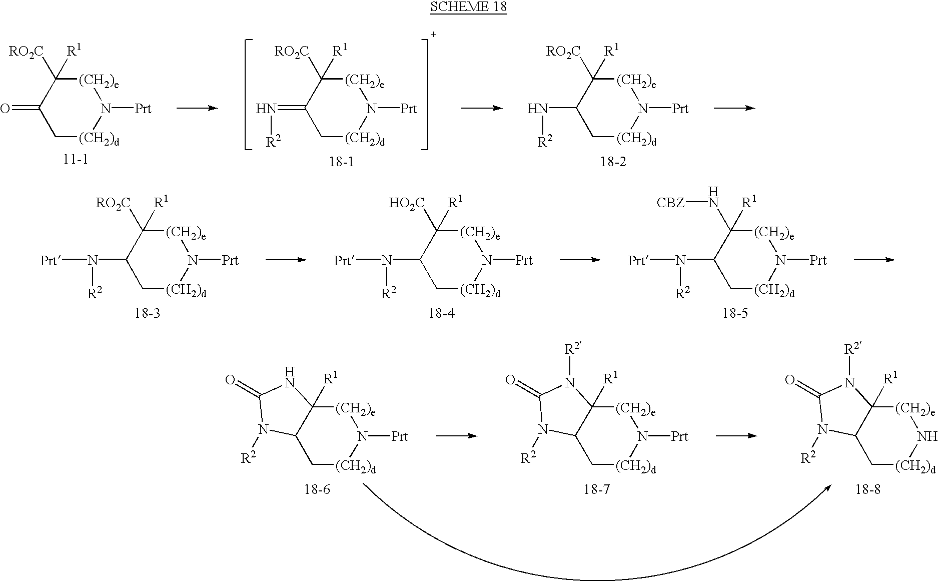

An intermediate amine of formula 18-2 can be prepared from a ketone of formula 11-1 (R is an alkyl group) by reductive amination as described above (see Scheme 8). Protection of the secondary amine in 18-2 produces 18-3. Intermediate carboxylic acids of formula 18-4 can be prepared by hydrolysis of the ester group of formula 18-3 (see Scheme 11). Transformation of 18-4 to 18-5 can be achieved through an intermediate acyl azide as described above (see Scheme 11). Cyclization of an intermediate of formula 18-5 at a suitable temperature after removing Prt′ yields an intermediate urea of formula 18-6. Deprotection of 18-6 provides 18-8 where R^{2′} is H. Alternatively, urea 18-6 can be alkylated by deprotonation with a strong base such as sodium hydride, LHMDS, or KHMDS in a suitable solvent such as DMF or THF followed by treatment with an alkylating agent such as an alkyl halide, mesylate or tosylate. Removal of the protecting group transforms 18-7 to 18-8 where R² and R^{2′} are each alkyl.

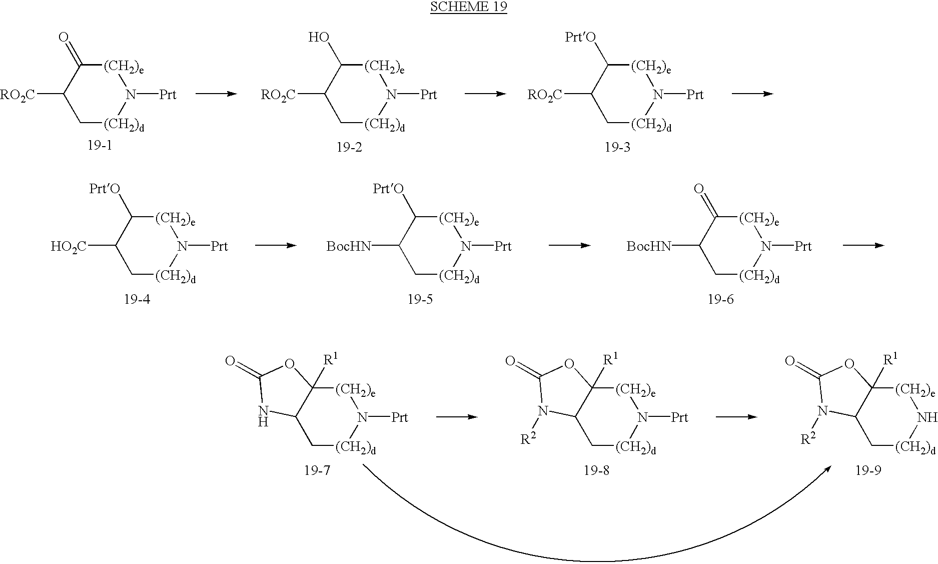

As illustrated in Scheme 19, reduction of a ketoester of formula 19-1, such as with sodium borohydride in methanol, preferably at 0 ° C., produces an alcohol of formula 19-2. An intermediate of formula 19-3 can be prepared by protection of the hydroxyl group in an intermediate of formula 19-2 with a suitable protecting group, such as forming a tetrahydropyranyl acetal or silyl ether. Transformation of the ester of formula 19-3 to amide 19-5 can be achieved as described above (see Scheme 11). Deprotection of the hydroxy group of 19-5 yields the free alcohol intermediate, which can be oxidized to an intermediate ketone of formula 19-6 with a suitable oxidizing agent, such as pyridinium chlorochromate or a Swern-type reagent (see Scheme 8).

Transformation of 19-6 to a cyclized carbamate of formula 19-7 can be achieved by treating 19-6 with an alkyl metal, such as a Grignard reagent, in a suitable solvent such as THF, followed by cyclization. Removal of the protecting group then yields 19-9 wherein R² is H. Alternatively, the carbamate of 19-7 may be alkylated as described above (see Scheme 13) to afford 19-8, which can then be deprotected to provide 19-9. Those skilled in the art will recognize that an R^{1A} substituent could have been introduced by alkylating ketoester 19-1.

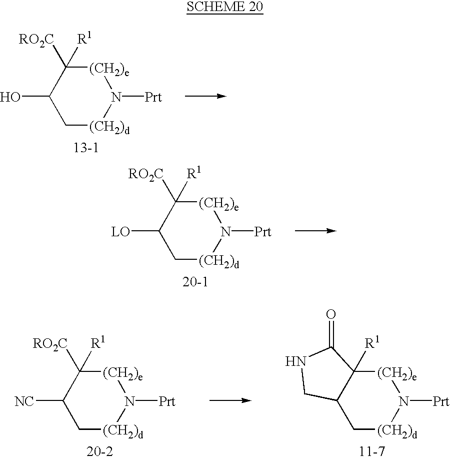

An alternate synthesis of lactam 11-7 is illustrated in Scheme 20. An alcohol of formula 13-1 can be converted to an intermediate nitrile of formula 20-2 by first activating the hydroxyl of 13-1 (R is an alkyl group), such as with methanesulfonyl chloride or methanesulfonic acid in a suitable solvent, such as methylene chloride in the presence of an amine base. Subsequent reaction of 20-1 (LO- is an activated hydroxyl) with a cyanide salt, such as potassium cyanide, then yields an intermediate nitrile of formula 20-2, which can be transformed to 11-7 by catalytic hydrogenation of the nitrile to amine, which then reacts with the ester group to form lactam (11-7). Those skilled in the art will recognize that an R^{1A} substituent could be introduced by alkylating nitrile 20-2.

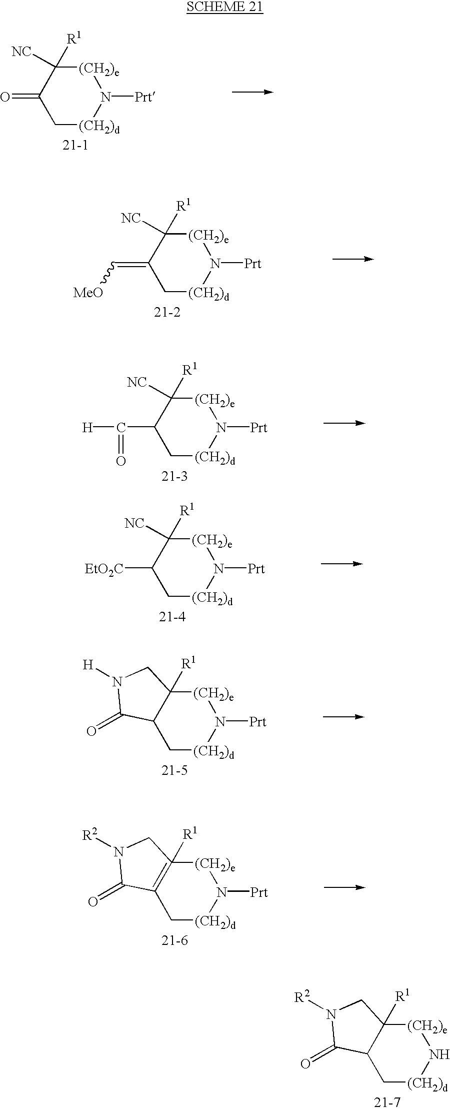

Nitriles of formula 21-1 can be prepared from esters, acid halides and acids of formula 11-1 by a variety of known methods (for examples, see R. Larock pages 976, 980 and 988 in Comprehensive Organic Transformations: A Guide to Functional Group Preparations, VCH Publishers, 1989).

Homologation of ketones of formula 21-1 to provide 21-3 as described above (Scheme 12) yields an aldehyde of formula 21-3. Oxidation of the aldehyde group in 21-3, such as with sodium hypochlorite, provides an acid which can be esterified to give 21-4 by a number of methods described above (Scheme 6). Reduction of the nitrile group in a compound of formula 21-4, such as by catalytic hydrogenation over Pd on carbon, gives an amine which will cyclize to give a lactam of formula 21-5. Deprotection of 21-5 yields 21-7, R² is H. Alternatively, alkylation of the amide of formula 21-5 as described above (Scheme 11) yields an N-substituted amide of formula 21-6, which can be deprotected to provide 21-7. Those skilled in the art will recognize that an R^{1A} substituent could have been introduced by alkylating ester 21-4.

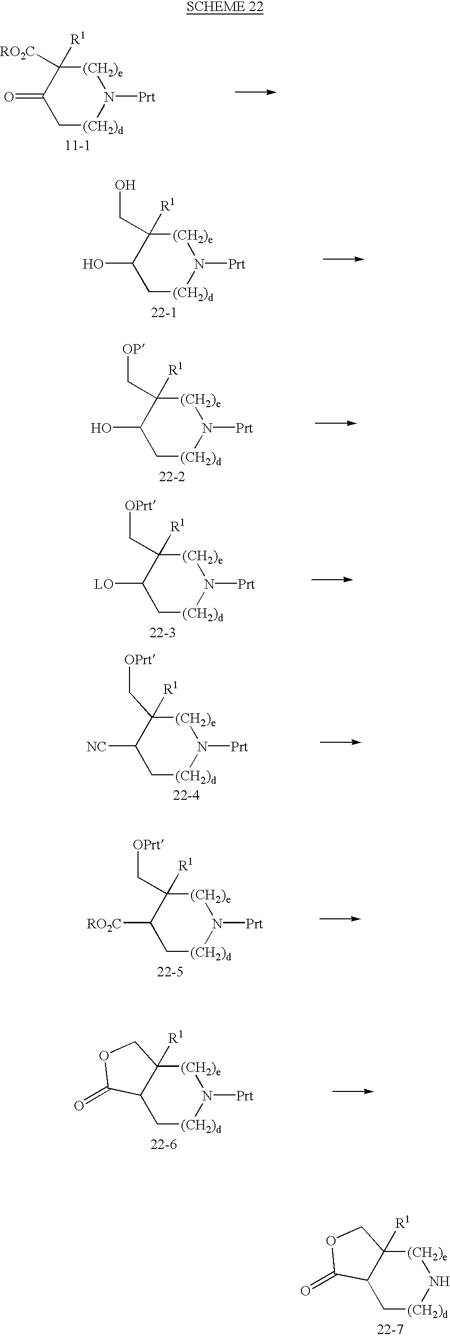

Intermediate alcohols of formula 22-1 can be prepared by reducing the ketone and ester groups of 11-1 (R is an alkyl group), such as with a metal borohydride or lithium aluminum hydride in a suitable solvent such as THF. Selective protection of the primary hydroxyl group of the intermediate of formula 22-1 with a suitable protecting group, such as a trialkylsilyl ether or pivaloyl ester gives a secondary alcohol of formula 22-2. An intermediate nitrile of formula 22-4 can be prepared from the alcohol of formula 22-2 by methods described above (see Scheme 20). An intermediate nitrile of formula 22-4 can be transformed to an ester of formula 22-5 by alcoholysis of nitrile 22-4, for instance with aqueous HCl or sodium hydroxide in ethanol. Removal of the alcohol protecting group and reaction of the hydroxyl group with the adjacent ester group in 22-5 forms a lactone of formula 22-6. Deprotection as described above yields 22-7. Those skilled in the art will recognize that an R^{1A} substituent could have been introduced by treating ketone 11-1 with the appropriate alkyl metal reagent. Substitution (R⁹, R¹⁰) adjacent to the lactone oxygen could then be introduced by treating the ester with the appropriate alkyl metal reagent (the ketone would have to be reduced if R^{1A} is not O).

[chemical structure]

Intermediate (α,β-unsaturated nitriles of formula 23-1 can be prepared by olefinating 11-1 (R is an alkyl group) with a reagent such as cyanomethyltriphenylphosphonium chloride and a strong base, such as KHMDS, in a suitable solvent, such as THF. Reduction of the double bond in 23-1, such as with sodium borohydride in pyridine, produces nitrile 23-2. The ester group of formula 23-2 can then be transformed to a carbamate of formula 234 by methods described above (see Scheme 11). Alcoholysis of the nitrile of 23-4 in an alcoholic solvent under acidic condition produces an ester of formula 23-5. A lactam of formula 23-6 can be prepared by removal of the CBZ protecting group, followed by cyclization of the amine with the adjacent ester group. Deprotection at this stage provides 23-8, R² is H. Alternatively, alkylation of the amide (according to Scheme 11) provides an N-substituted lactam, which can be converted to 23-8 by deprotection as described above. One skilled in the art will recognize that an R^{1A} substituent could have been introduced by conjugate addition to the unsaturated nitrile (23-1), such as with an alkyl cuprate. In addition, R⁹, R¹⁰ substituents can be introduced next to the lactam carbonyl by alkylating nitrile 23-2.

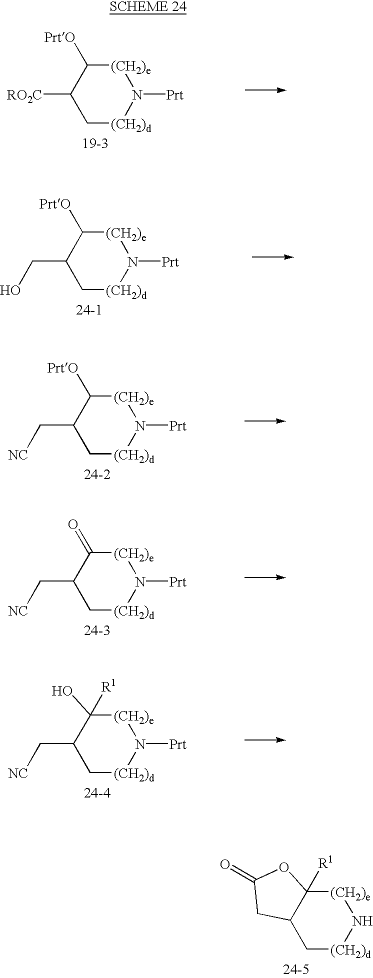

As illustrated in Scheme 24, an alcohol of formula 24-1 can be prepared from 19-3 (R is an alkyl group) by reduction of the ester with a reducing reagent such as lithium borohydride in a solvent such as THF. A nitrile of formula 24-2 can be prepared from the alcohols of formula 24-1 by methods described above (see Scheme 20). Deprotection of the alcohol of 24-2 followed by oxidation of the hydroxyl as previously described (see Scheme 19) produces a ketone 24-3. Treating 24-3 with an alkyl metal such as a Grignard reagent in a suitable solvent such as THF gives an intermediate of formula 24-4. The cyano group of 24-4 can then be converted to an ester by alcoholysis as described above (Scheme 22). Reaction of the tertiary alcohol with the neighboring ester forms a lactone which can then be deprotected to give 24-5. One skilled in the art will recognize that an R^{1A} substituent could be introduced by alkylating ester 19-3. In addition, R⁹, R¹⁰ substituents could be introduced adjacent to the lactone carbonyl by alkylation before final deprotection.

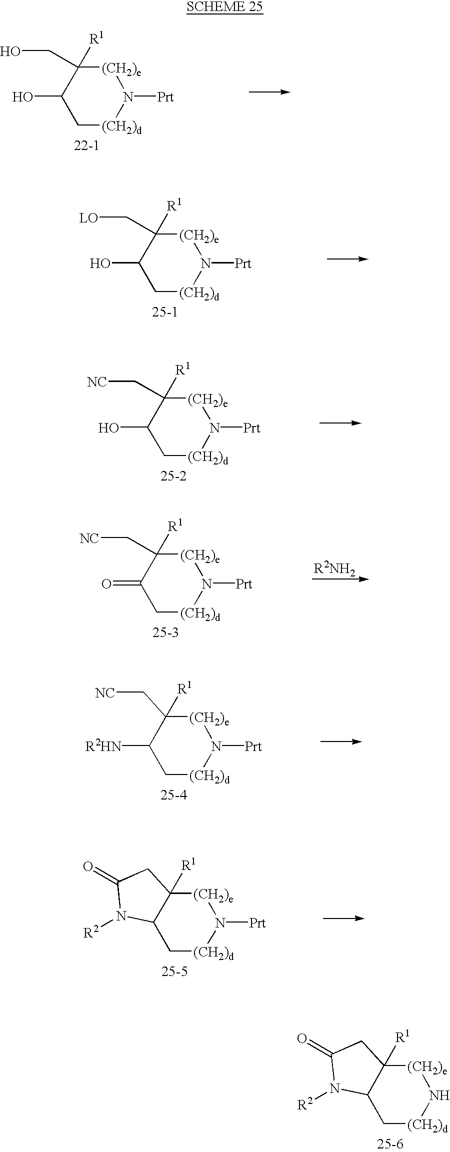

Intermediate of formula 25-1 (LO- is an activated hydroxyl) can be prepared by selective activation of the primary hydroxyl, for instance by tosylation of the less hindered hydroxyl group of 20-1 with tosyl chloride in a suitable solvent. Treating 25-1 with a reagent such as potassium cyanide in a suitable solvent produces a nitrile of formula 25-2. Oxidation of the alcohol (see Scheme 19) of formula 25-2 gives a ketone of formula 25-3. Transformation of 25-3 to 254 can be achieved by reductive amination as was described above (see Scheme 8). The cyano amine of formula 25-4 can be converted to a lactam of formula 25-5 by treating 254 with a strong acid or base in a protic solvent such as ethanol. Removal of the protecting group on the secondary nitrogen can then provide lactam 25-6. One skilled in the art will recognize that R⁹, R¹⁰ substituents could be introduced by alkylation of lactam 25-5.

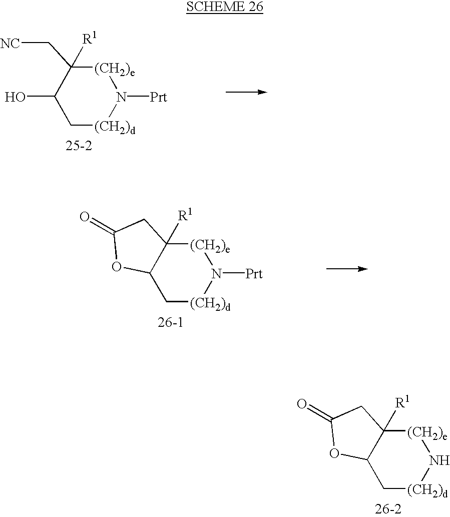

A lactone of formula 26-1 can be prepared by treating a cyano alcohol of formula 25-2 with a strong acid such as HCl, or a strong base such as NaOH, in a protic solvent such as EtOH. Deprotection, as described above, of the secondary amine of formula 26-1 gives 26-2. One skilled in the art will recognize that R⁹, R¹⁰ substituents can be introduced by alkylation of lactone 26-1.

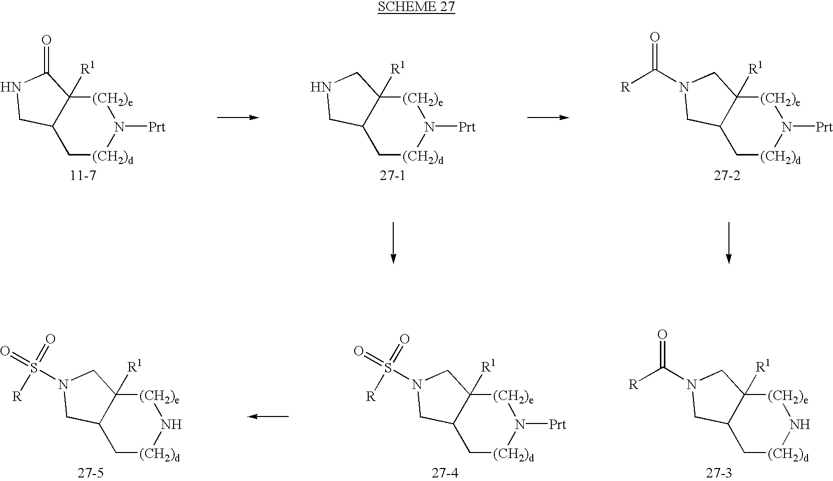

Intermediates of formula 27-1 can be prepared by reducing a lactam of formula 11-7 to a pyrrolidine with a suitable reducing reagent such as borane or lithium aluminum hydride in a suitable solvent such as THF . Treating 27-1 with an acyl chloride of formula RCOCI (where R is an alkyl group) in a suitable solvent produces an intermediate amide of formula 27-2. Removal of the protecting group of the amide of formula 27-2 by the method described previously gives an amide of formula 27-3.

A sulfonamide of formula 27-5 can be prepared by treating 27-1 with a sulfonyl halide such as tosyl chloride in the presence of a base such as pyridine to yield 27-4, followed by removal of the protecting group as previously described.

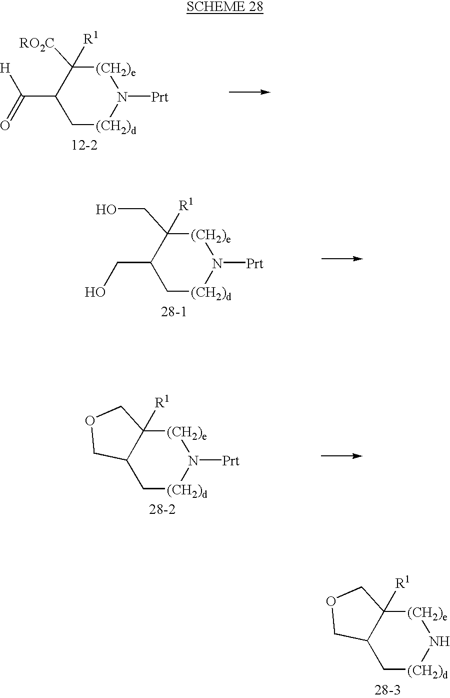

Intermediate diols of formula 28-1 (R is an alkyl group) can be prepared by treating 12-2 with a suitable reducing agent, such as lithium borohydride, in an appropriate solvent, such as THF. Methods for converting diol 28-1 to furan 28-2 include dehydration under acidic conditions, dehydration with a reagent such as Ph₃P(OEt)₂, or reaction with a reagent such as toluenesulfonylchloride in the presence of a base followed by displacement of the activated alcohol with the remaining hydroxyl group. Removal of the protecting group from 28-2 subsequently forms a compound of formula 28-3. One skilled in the art will recognize that an R^{1A} substituent can be added by alkylating aldehyde 12-2. In addition, R⁹, R¹⁰ substituents can be introduced by treating 12-2 with an alkyl metal reagent.

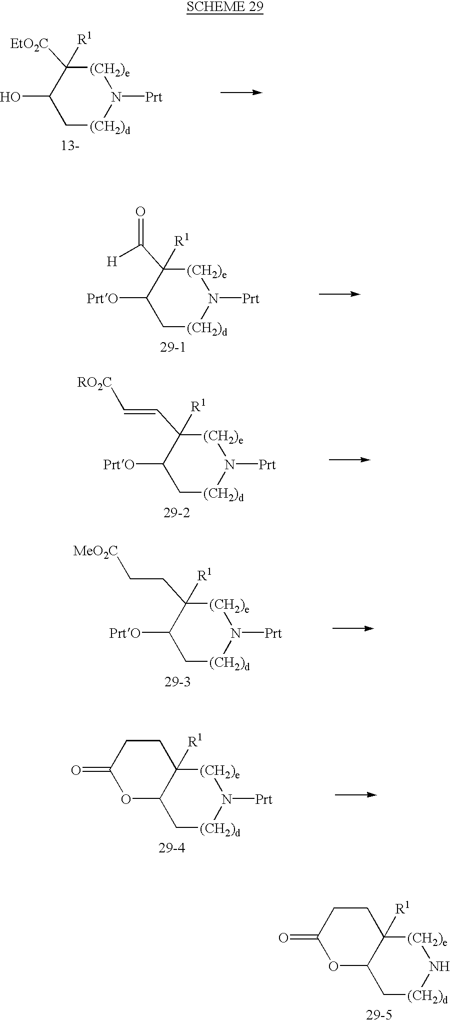

Intermediate aldehydes of formula 29-1 can be prepared by protecting the secondary alcohol of 13-1 such as with a silyl ether, followed by reduction of the ester with a reducing reagent such as diisobutylaluminum hydride at −78 ° C. in a suitable solvent. Alternatively, 13-1 can be reduced to the primary alcohol with a reagent such as lithium borohydride, and then oxidized to the aldehyde with a variety of reagents described above (see Scheme 8). Homologation of aldehydes of formula 29-1 to saturated esters of formula 29-3 can be performed as previously described (see similar homologation of ketones in Scheme 11). Deprotection of the secondary alcohol of 29-3, followed by cyclization produces lactones of formula 29-4. Deprotection of 29-4 will then give 29-5. An R⁹ substituent β to the lactone carbonyl may be introduced by conjugate addition to unsaturated ester 29-2, such as with an alkyl cuprate. In addition, R⁹, R¹⁰ substituents could be introduced next to the lactone carbonyl by alkylating lactone 29-4.

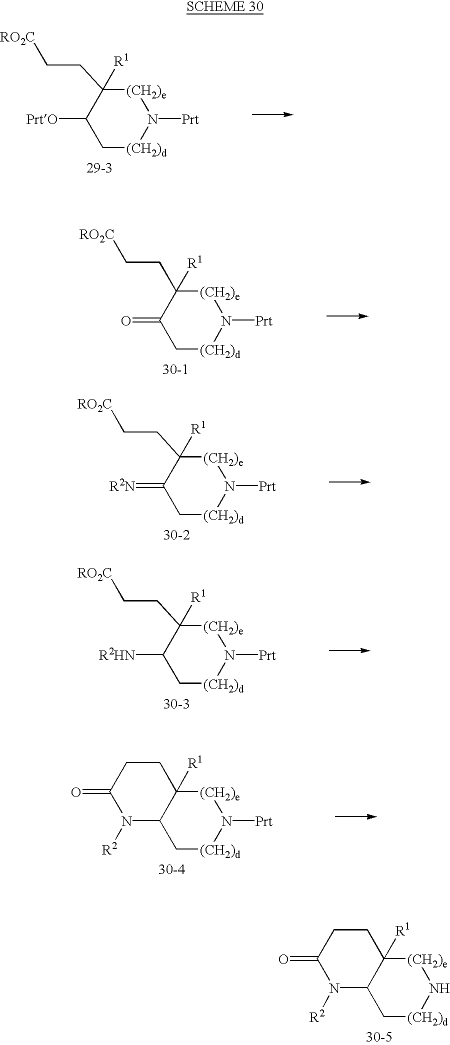

Intermediate ketones of formula 30-1 can be prepared by deprotecting the secondary hydroxyl of 29-3 (R is an alkyl group), followed by oxidation of the alcohol to a ketone (see Scheme 19). Reductive amination of 30-1 with a primary amine as previously described (see Scheme 8) produces intermediate 30-3. Cyclization of 30-3 at a suitable temperature yields a lactam of formula 30-4, which can be deprotected to give 30-5. One skilled in the art will recognize that R⁹, R¹3 substituents can be introduced by alkylation of lactam 30-4.

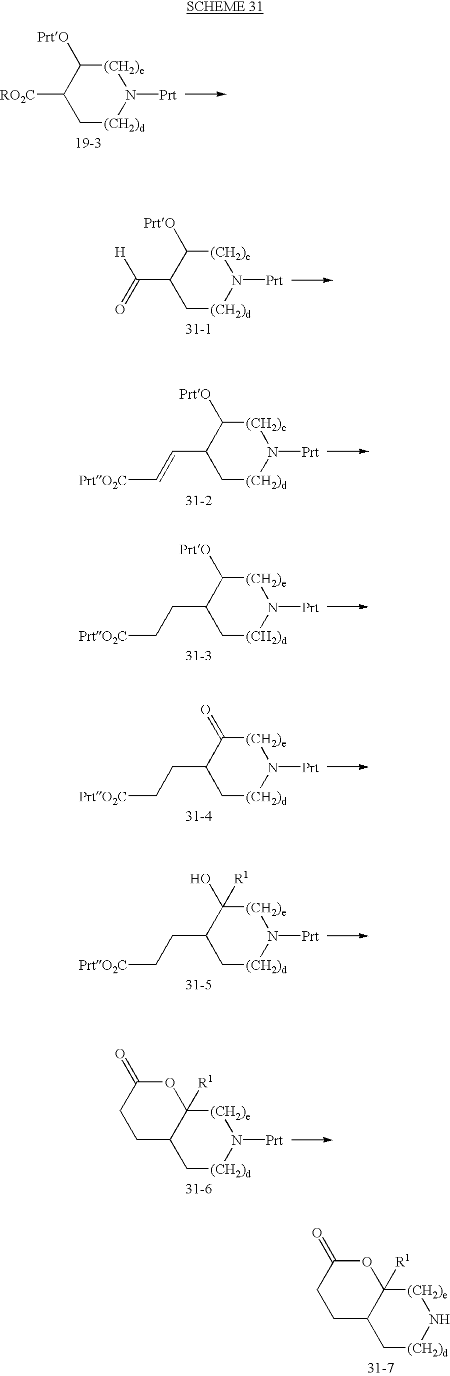

Homologation of 19-3 (R is an alkyl group) to an ester of formula 31-3 can be performed analogously to routes described above (see Scheme 29). Removal of Prt′ of 31-3 gives a secondary alcohol which can be oxidized as was previously described (see Scheme 19) to produce a ketone of formula 31-4. Treating 31-4 with an alkyl metal reagent, such as a Grignard reagent, in a suitable solvent produces intermediate 31-5, which can be cyclized to form lactone 31-6. Removal of the protecting group then produces 31-7. One skilled in the art will recognize that an R^{1A} substituent may be introduced by alkylation of ester 19-3. A substituent β to the lactone carbonyl may be introduced by conjugate addition to unsaturated ester 31-2, such as with an alkyl cuprate. Also, R⁹, R¹⁰ substituents can be introduced next to the lactone by alkylation of 31-6.

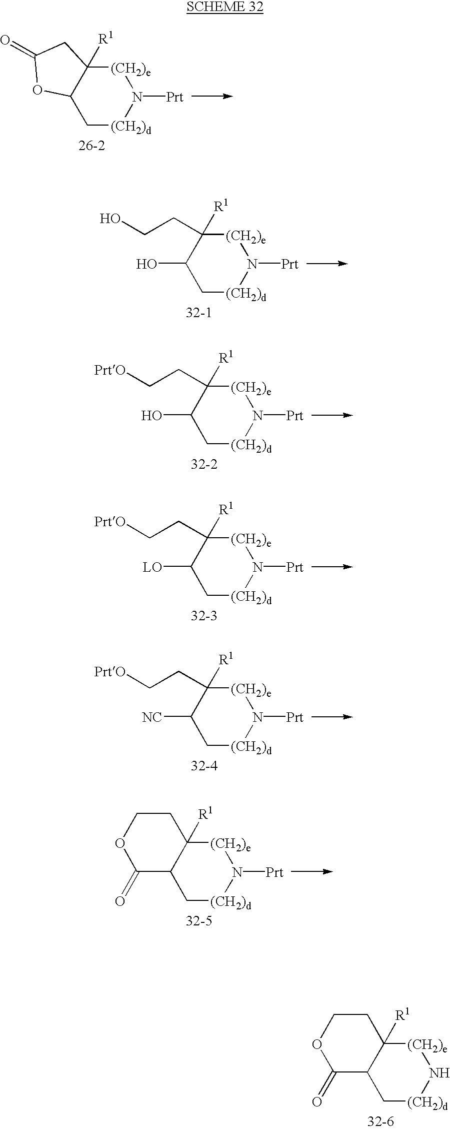

Intermediate diols of formula 32-1 can be prepared by reducing the lactone group of 26-2 with a reagent such as lithium aluminum hydride in a suitable solvent such as THF at a suitable temperature. Selective protection at the less hindered hydroxy group of 32-1, such as with t-butyldimethylsilyl chloride using triethylamine in the presence of DMAP in a solvent such as dichloromethane, produces alcohol 32-2. Conversion of alcohol 32-2 to a nitrile of formula 324 may be accomplished as described above (LO- is an activated hydroxyl group) (see Scheme 20). Alcoholysis of the cyano group of formula 32-4 (see Scheme 22), deprotection of the alcohol, and subsequent lactonization forms lactones of formula 32-5. Deprotection of an amine of formula 32-5 gives a lactone of formula 32-6. One skilled in the art will recognize that R⁹, R¹⁰ substituents can be introduced β- to the ring oxygen in lactone 32-6 by alkylating lactone 26-2. Substitution β to the lactone ring oxygen may be introduced by treating 26-2 with an alkyl metal reagent.

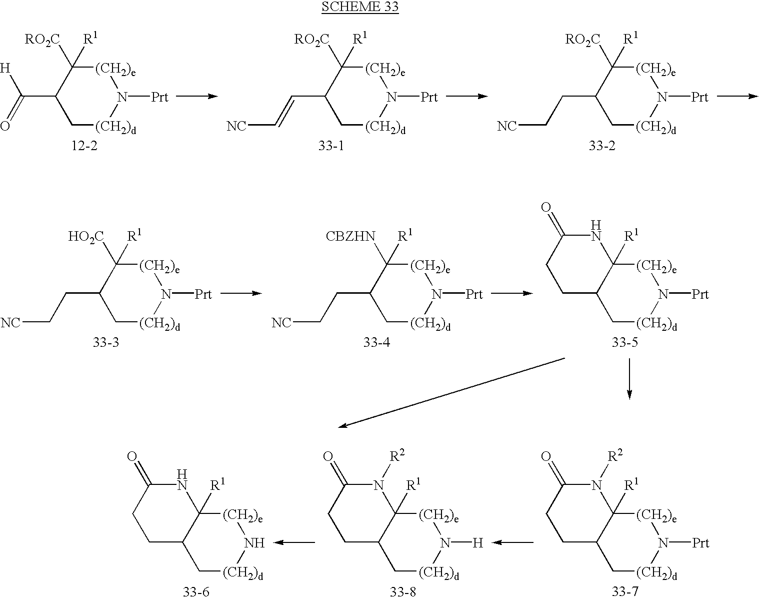

Intermediate nitriles of formula 33-2 can be prepared by homologating 12-2 (R is an alkyl group), analogous to the ketone homologation described in Scheme 23. Conversion of ester 33-2 to carbamates of formula 33-4 can be accomplished as described above (see Scheme 11). Alcoholysis of the cyano group of 33-4 as described above (see Scheme 22) and removal of the CBZ protecting group, followed by cyclization of the amine with the adjacent ester group produces a lactam of formula 33-5. Deprotection of 33-5 gives the lactam of formula 33-6.

Alternatively, alkylation of 33-5 in the usual fashion (see Scheme 11) gives 33-7, which can be deprotected to give 33-8. One skilled in the art will recognize that an R^{1A} substituent may be introduced by alkylating aldehyde 12-2. An R⁹ substituent may be introduced by conjugate addition to the unsaturated nitrile (33-1). R⁹, R¹⁰ substitution can be introduced next to the lactam by alkylation of 33-7.

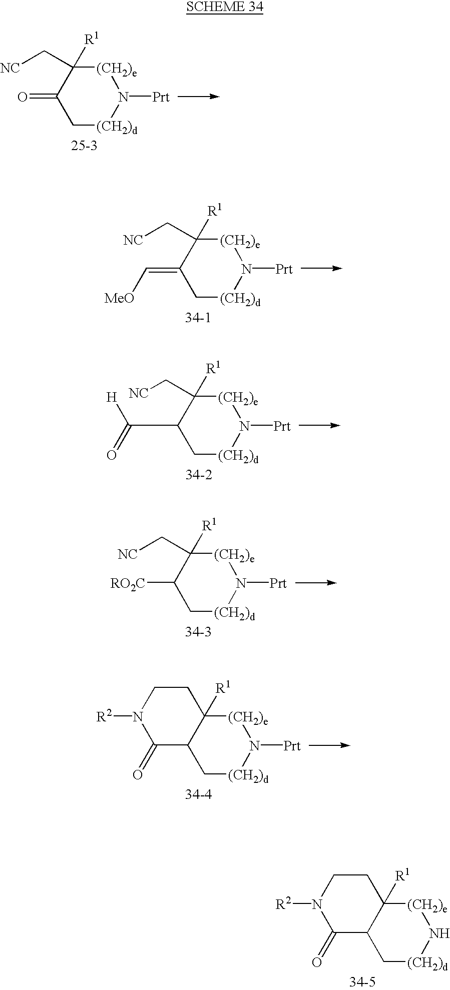

The homologation of 25-3 to give a lactam of formula 34-5 can be analogously performed according to the procedures described in Scheme 21. One skilled in the art will recognize that an R^{1A} substituent may be introduced by alkylating 34-4 (R is an alkyl group). R⁹, R¹⁰ substitution may be introduced by alkylating nitrile 34-1.

[chemical structure]

As illustrated in Scheme 35, catalytic hydrogenation of a nitrile of formula 23-2 (R is an alkyl group) gives an amine, followed by cyclization of the amine with the adjacent ester group to give lactams of formula 35-1. Deprotection of 35-1 gives 35-3, R² is H. Alternatively, alkylation of lactam 35-1 as described above (see Scheme 11) provides N-substituted amides of formula 35-2. Deprotection of 35-2 affords 35-3. One skilled in the art will recognize that an R^{1A} substituent may be introduced by conjugate addition to the unsaturated nitrile.

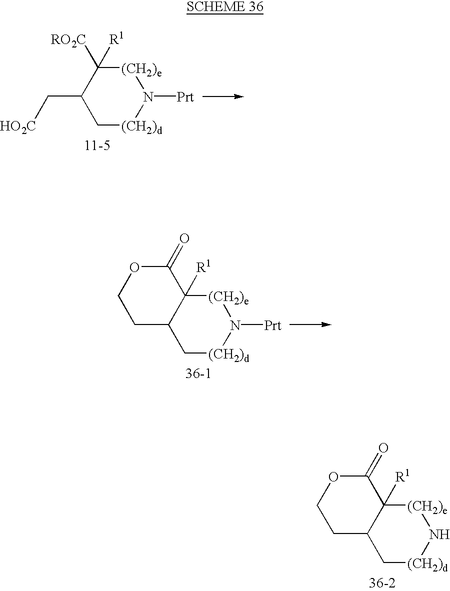

As illustrated in Scheme 36, selective reduction of the carboxylic acid group of 11-5 to an alcohol, such as by treating 11-5 (R is an alkyl group) with borane in a suitable solvent, followed by cyclization of the alcohol and ester produces a lactone of the formula 36-1. Deprotection of 36-1 then gives 36-2.

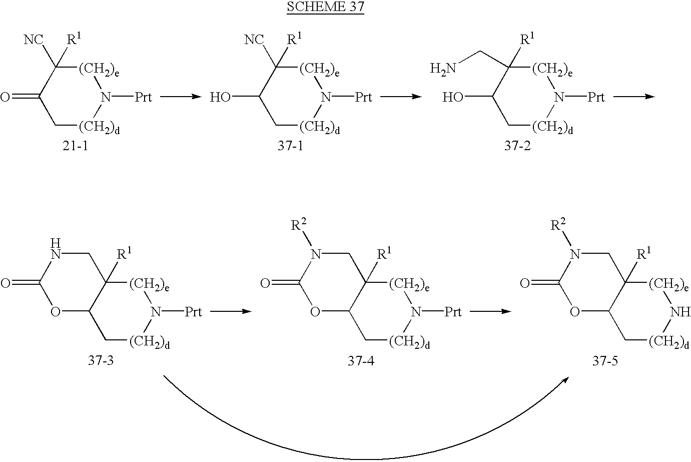

Intermediate alcohols of formula 37-1 can be prepared by reducing the ketone of 21-1, such as with sodium borohydride in a solvent such as methanol at a temperature of about 0° C. Reduction of the cyano group to an amine, such as by catalytic hydrogenation, affords aminoalcohol 37-2. Treating 37-2 with a reagent like CDl or other phosgene equivalent in the presence of a base like TEA (see Scheme 14) produces a cyclized carbamate of formula 37-3. Deprotection of 37-3 then gives 37-5, R² is H. Alternatively, 37-3 may be alkylated as described above (see Scheme 13) to give an N-substituted carbamate of formula 37-4, which is deprotected to give 37-5. One skilled in the art will recognize that an R^{1A} substituent may be introduced by addition to ketone 21-1.

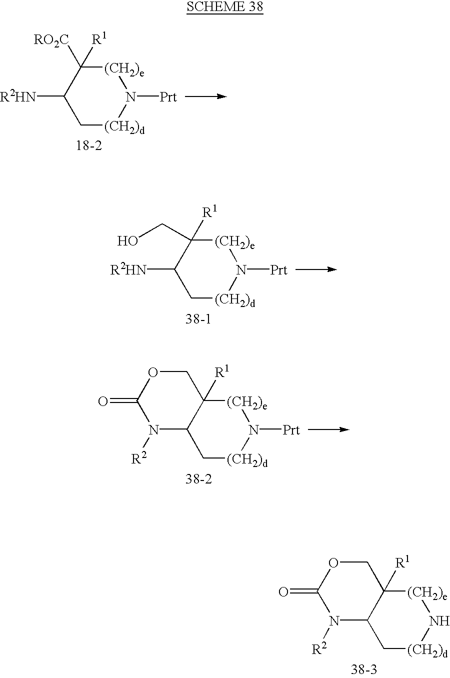

Intermediate aminoalcohols of formula 38-1 can be prepared by reducing an ester of formula 18-2 (R is an alkyl group), such as with lithium borohydride. Treating 38-1 with a phosgene equivalent as described in Scheme 14 produces a cyclized carbamate of formula 38-2. Deprotection subsequently provides 38-3.

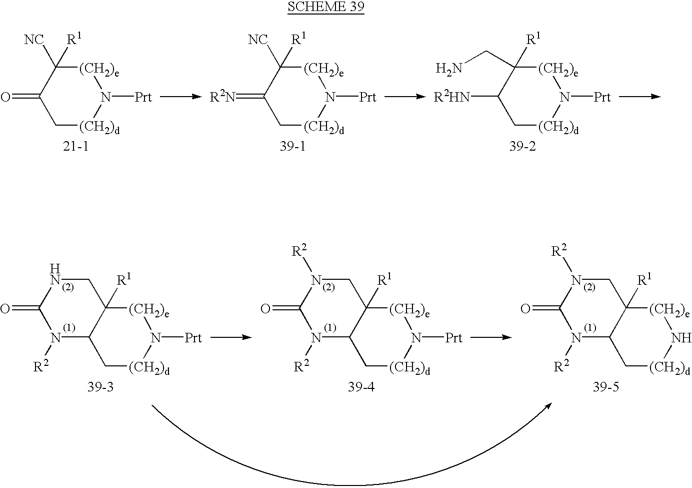

Intermediate imines of formula 39-1 can be prepared by condensing the ketone of 21-1 with a primary amine under dehydrating conditions, such as azeotropic distillation using a solvent like benzene. Catalytic hydrogenation to reduce the nitrile and imine converts 39-1 to 39-2. Treating 39-2 with a reagent like CDl, phosgene, or triphosgene in the presence of a base like TEA produces the cyclized and N-substituted ureas of formula 39-3. Deprotection of this material provides 39-5 where the R² attached to the (2)-nitrogen is H. Alkylation of 39-3, such as with sodium 5 hydride and an alkyl halide produces the N,N′-substituted ureas of formula 39-4, which can be deprotected to provide 39-5 where the R² attached to the (2)-nitrogen is an alkyl group.

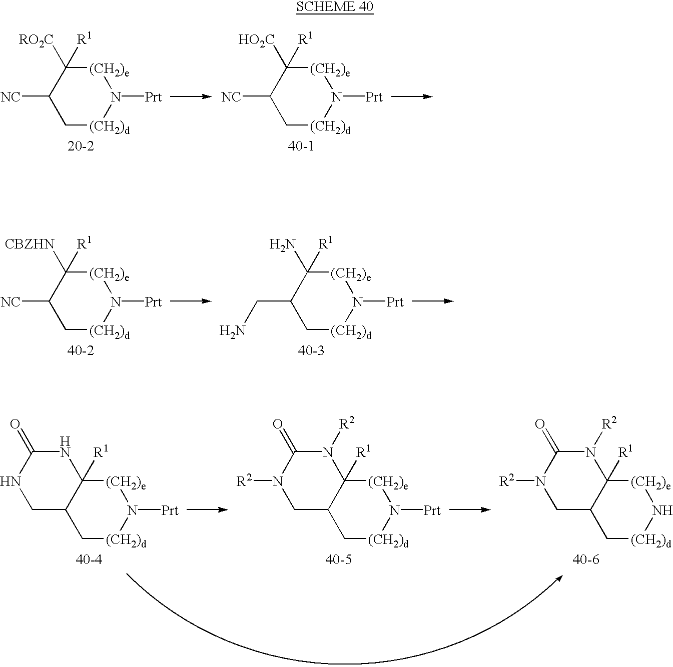

As illustrated in Scheme 40, ester 20-2 (R is an alkyl group) can be converted to carbamate 40-2 as described above (see Scheme 1 1). Catalytic hydrogenation of 40-2 will reduce the nitrile and cleave the CBZ group to provide a diamine of formula 40-3. Acylating 40-3 with a reagent such as CDl, phosgene, or triphosgene in the presence of a base like TEA produces the cyclized ureas of formula 40-4. Deprotection at this stage provides 40-6 where each R ² is H. Alternatively, alkylation of 40-4, such as by deprotonation with a strong base like sodium hydride followed by reaction with an alkylating reagent like an alkyl halide, tosylate or mesylate produces the N,N′-substituted ureas of formula 40-5. Deprotection then provides 40-6 where each R² is alkyl. One skilled in the art will recognize that an R^{1A} substituent may be introduced by alkylation of nitrile 20-2.

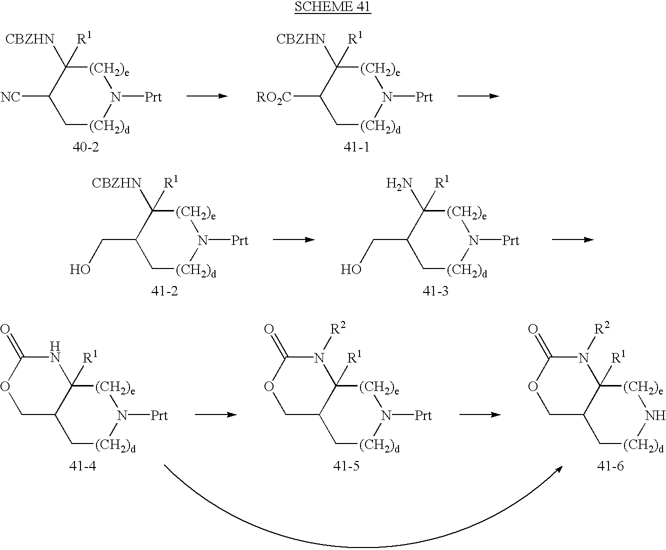

Intermediate esters of formula 41-1 (R is an alkyl group) can be prepared by alcoholysis of the cyano group in 40-2 with ethanolic HCl. Reducing the ester group in 41-1, such as with lithium borohydride in THF produces an alcohol of formula 41-2. Catalytic hydrogenation to remove the CBZ group to yield an amine as previously described converts 41-2 to 41-3. Treating 41-3 with a reagent like CDl or other phosgene equivalent in the presence of a base like TEA produces a carbamate of formula 41-4. Deprotection at this stage provides 41-6 where R²is H. Alternatively, transformation of 41-4 to N-substituted carbamates of formula 41-5 can be achieved by deprotonating 41-4 with a strong base such as sodium hydride in a solvent like DMF, followed by alkylation with a reagent such as an alkyl halide, tosylate or mesylate. Deprotection then converts 41-5 to 41-6 where R² is alkyl.

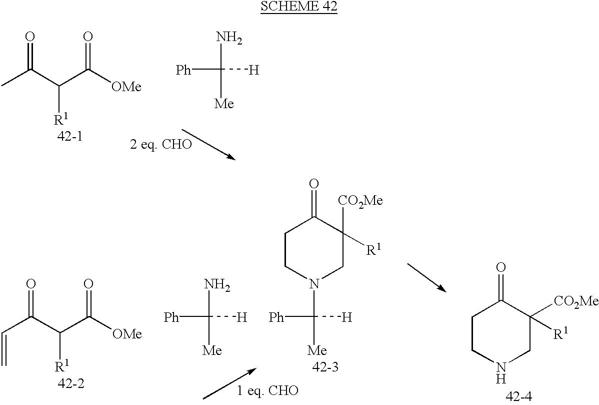

Reaction of a ketoester of formula 42-1 with a chiral amine such as alpha-methylbenzylamine with a suitable aldehyde such as formaldehyde, or reaction of a vinyl ketoester of formula 42-2 with a chiral amine such as alpha-methylbenzylamine with a suitable aldehyde such as formaldehyde, affords a compound of formula 42-3 via a double Mannich reaction. Compound 42-3 is equivalent to 11-1 where d and e are 1, and may be deprotected with a suitable catalyst such as palladium in the presence of hydrogen to give 42-4. In addition, 42-3 could be isolated as a single diastereomer (by selective cyclization or separation of diastereomers), thereby providing 42-4 as a single enantiomer.

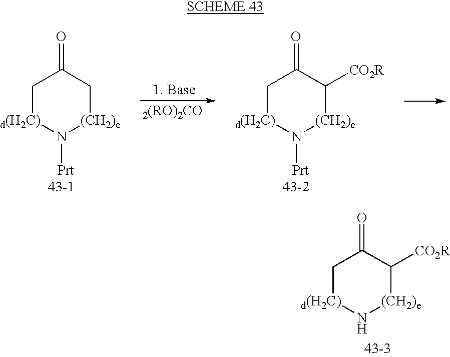

Treatment of a compound of formula 43-1 with a base such as sodium hydride in a solvent such as DMF followed by treatment with diethylcarbonate generates the ethyl ester of compound 43-2 (R is an alkyl group). Deprotection of the amine transforms 43-2 into 43-3. It will be recognized by one skilled in the art that 19-1 is equivalent to 43-3.

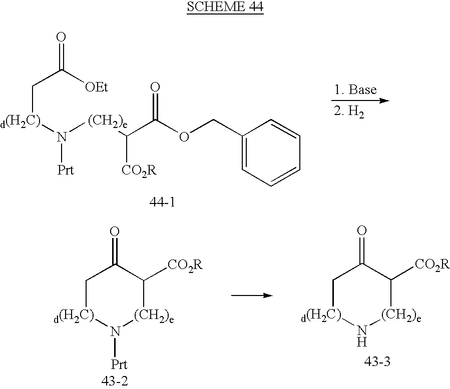

Treatment of a malonic ester of formula 44-1 (R is an alkyl group) with a base such as sodium hydride in a solvent such as DMF and subsequent hydrogenolysis of the benzyl group with hydrogen and a catalyst such as palladium in a suitable solvent such as methanol produces the ester of formula 43-2. Deprotection of the amine generates compounds of formula 43-3. It will be recognized by one skilled in the art that 19-1 is equivalent to 43-3.

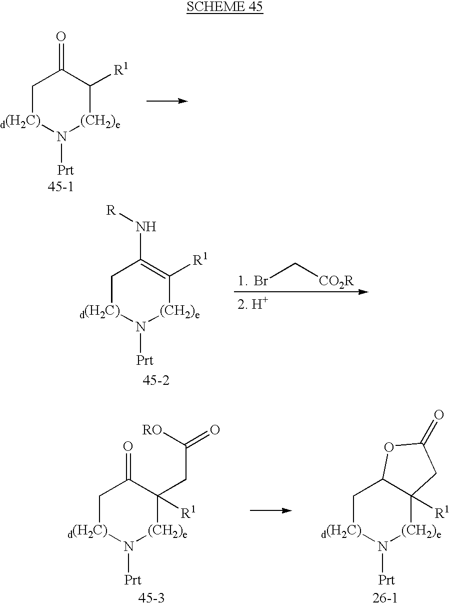

Treatment of a ketone of formula 45-1 with a secondary amine such as piperidine in a suitable solvent such as benzene with removal of water affords an enamine of formula 45-2 (each R is an alkyl group). Alkylation of the enamine with an alpha-haloester such as ethylbromoacetate in a suitable solvent such as benzene or THF using a suitable base such as LDA or NaN(SiMe₃)₂ affords a ketoester of formula 45-3. Reduction with a mild reducing agent such as sodium borohydride in methanol and subsequent cyclization then affords 26-1.

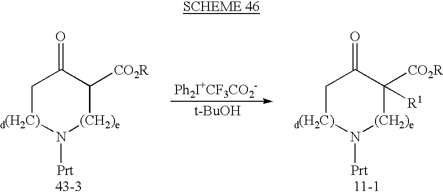

Treatment of a ketoester of formula 43-3 (R is an alkyl group) with an iodonium salt such as diphenyliodonium trifluoroacetate in a suitable solvent such as t-butanol generates a ketoester of formula 11-1 where R¹ is phenyl. See Synthesis, (9), 1984 p. 709 for a detailed description.

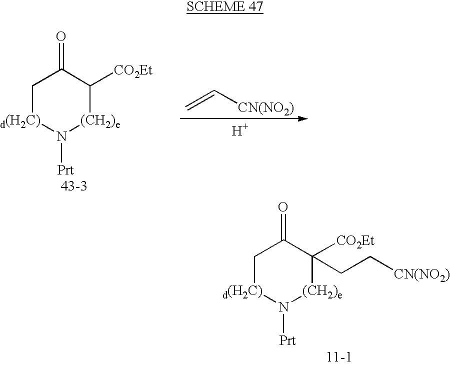

Treatment of a ketoester of formula 43-3 with an olefin such as acrylonitrile or nitroethylene generates a ketoester of formula 11-1 where R¹ is CH₂CH₂CN or R¹ is CH₂CH₂NO₂.

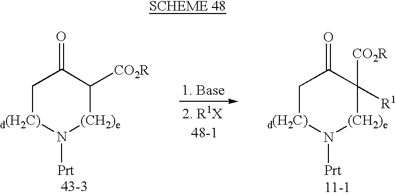

Treatment of an ester of formula 43-3 (R is an alkyl group) with a base such as sodium hydride in a solvent such as DMF followed by an alkyl halide 48-1 generates a compound of formula 11-1 as illustrated in Scheme 48.

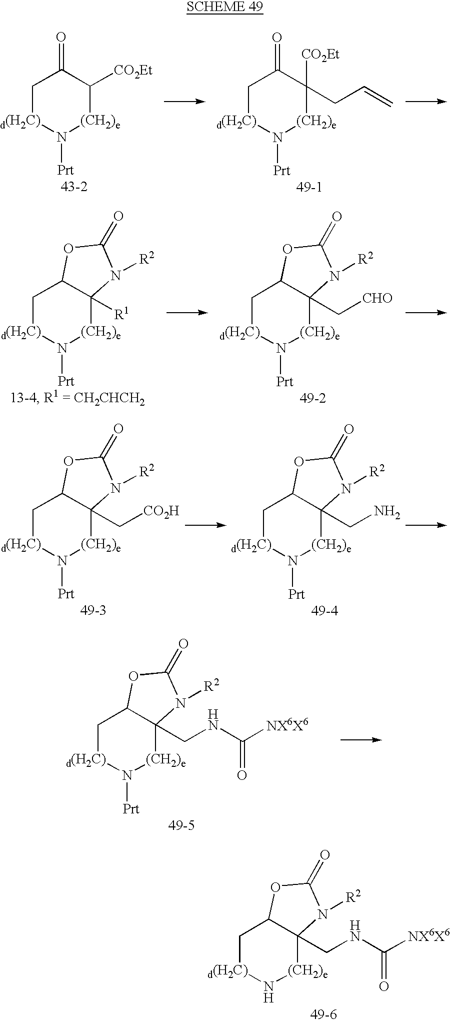

Treatment of a ketoester of formula 43-2 with allyl bromide and a suitable base such as sodium hydride in a suitable solvent such as DMF affords a ketoester of formula 49-1 (11-1, R² is allyl). Compound 49-1 may then be converted to 13-4 as described in Scheme 13. Ozonolysis of 13-4 in a suitable solvent such as methylene chloride followed by treatment with a reducing agent such as dimethylsulfide affords an aldehyde of formula 49-2. Oxidation of 49-2 affords a carboxylic acid of formula 49-3. Curtius rearrangement of 49-3, followed by hydrolysis of the intermediate isocyanate affords a primary amine of formula 49-4. Treatment of a compound of formula 49-4 with an isocyanate or carbamate affords a urea of formula 49-5. Deprotection of the nitrogen affords compounds of formula 49-6 (e.g., 13-5, wherein R¹ is CH₂NHCONX⁶X⁶). Those skilled in the art will recognize that other heterocycles, prepared in previous schemes, could be transformed analogously to the conversion of 13-4 to 49-6.

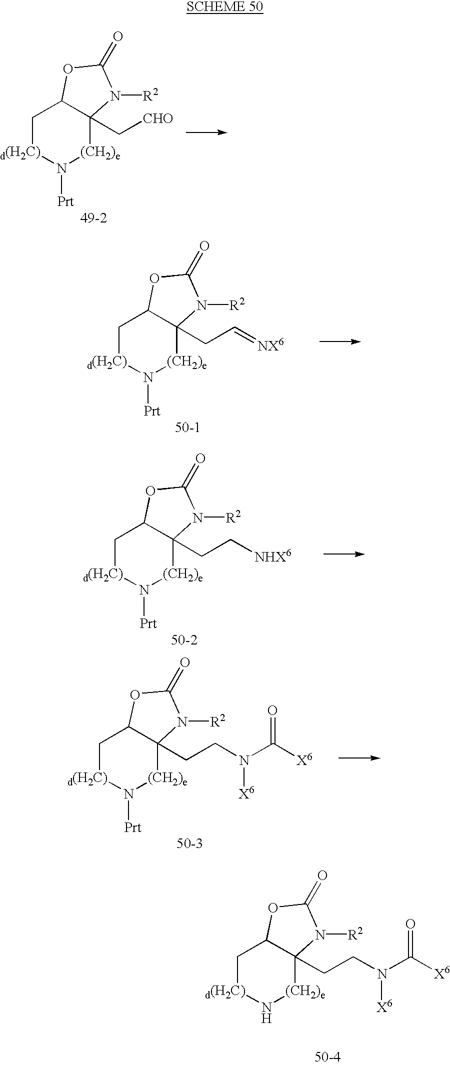

Treatment of a compound of formula 49-2 with a primary amine of formula HNX⁶ affords an imine of formula 50-1. Reduction of a compound of formula 50-1 affords a compound of formula 50-2. Treatment of a compound of formula 50-2 with an acylating agent affords a compound of formula 50-3. Deprotection of the nitrogen affords compounds of formula 50-4 (13-5, R¹ is CH₂CH₂NX⁶COX⁶). Those skilled in the art will recognize that other heterocycles, prepared in previous schemes, could be transformed in a manner analogous to the conversion of 49-2 to 50-4.

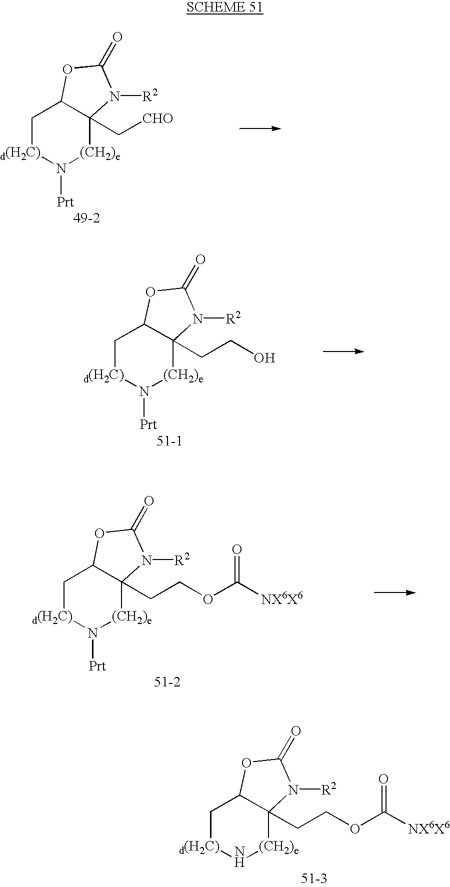

Treatment of a compound of formula 49-2 with a reducing agent such as sodium borohydride affords a compound of formula 51-1. Reaction of 51-1 with an acylating agent such as an isocyanate or carbamate affords compounds of formula 51-2. Deprotection of the nitrogen affords compounds of formula 51-3. Those skilled in the art will recognize that other heterocycles, prepared in previous schemes, could be transformed in a manner analogous to the conversion of 49-2 to 51-3.

[chemical structure]

Treatment of a compound of formula 51-1 with a phosphine such as triphenyl phosphine and an azo compound such as diethylazodicarboxylate and an oxindole affords a compound of formula 52-1. Deprotection of the nitrogen affords the compound of formula 52-3. Those skilled in the art will recognize that other heterocycles, prepared in previous schemes, could be transformed in a manner analogous to the conversion of 49-2 to 52-3.

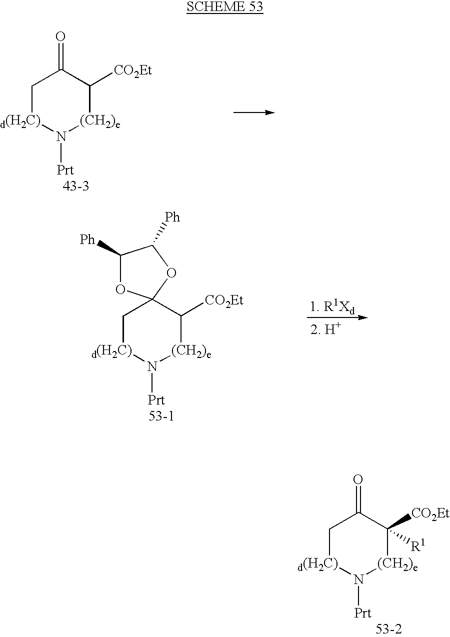

Treatment of a ketoester of formula 43-3 with a chiral diol and acid catalyst with removal of water in a suitable solvent such as benzene affords a chiral ketal like formula 53-1. Alkylation of 53-1 with an alkyl halide in the presence of a base such as LDA followed by acid-catalyzed hydrolysis of the ketal affords chiral ketoesters of formula 53-2. Ketoester 53-2 is a single enantiomer of 11-1 and may be homologated in a similar fashion to give various heterocycles.

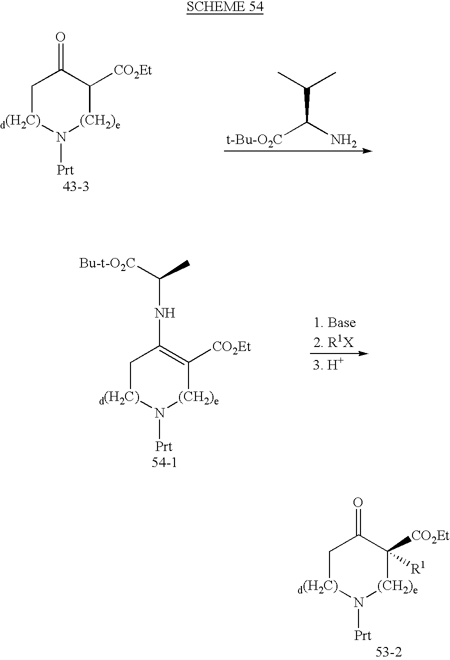

Treatment of a ketoester of formula 43-3 with a chiral amino acid ester such as valine t-butyl ester affords a chiral enamine of formula 54-1. Alkylation of 54-1 with an alkyl halide in the presence of a base such as LDA followed by acid-catalyzed hydrolysis of the enamine affords chiral ketoesters of formula 53-2.

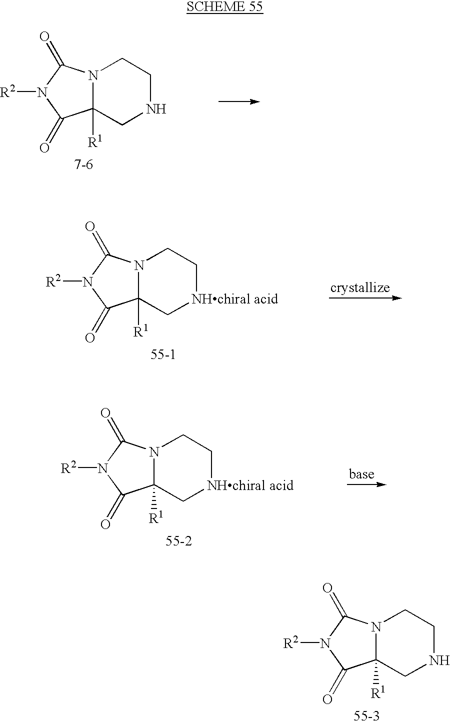

Salt formation of 7-6 with a chiral acid affords a mixture of diastereomeric salts of formula 55-1. Crystallization of the diastereomeric salts affords the acid salt of chiral compounds of formula 55-2. Decomposition of the salt 55-2 with base liberates chiral compounds of formula 55-3. This resolution scheme could be applied to the resolution of other HET-bicyclic compounds described above.

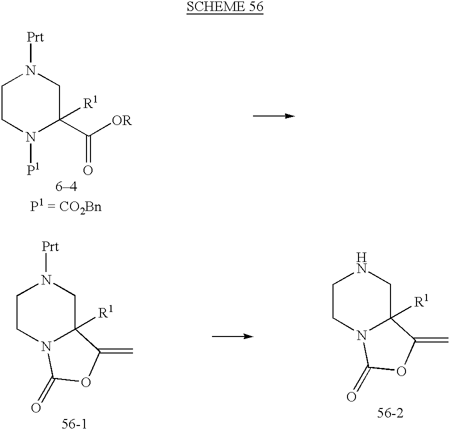

As illustrated in Scheme 56, treatment of 6-4 (P¹ is CO₂Bn) with an alkyl metal reagent like methyl magnesium bromide affords 56-1. Deprotection as usual then affords 56-2.

[chemical structure]

Compounds of formula 57-3 can be prepared from known phthalic or homophthalic anhydrides by methods previously described by Welch, Willard M. (J.Org.Chem 47; 5; 1982; 886-888. J.Org.Chem.; 47; 5; 1982; 886-888) or Machida, Minoru et al. (Heterocycles; 14; 9; 1980; 1255-1258). Alternatively, the analogous phthalimides or homophthalimides of formula 57-1 can be treated with the appropriate hydride reagent (e.g., NaBH₄) or organometallic reagent (e.g., methyl Grignard), followed by treatment with sodium or potassium cyanide to produce an intermediate of the formula 57-2. Compounds of formula 57-2 can be converted to compounds of formula 57-3 as previously described by Welch, Willard M. (J.Org.Chem 47; 5; 1982; 886-888).

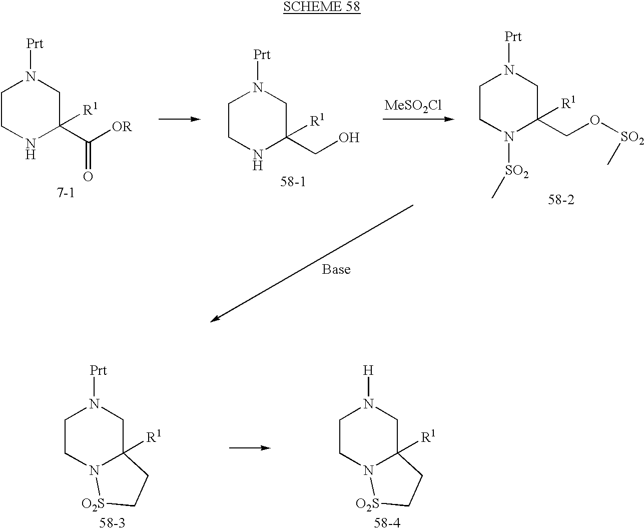

As illustrated in Scheme 58, intermediates of formula 58-4 can be prepared in four steps from compounds of formula 7-1. Compounds of formula 7-1 are treated with a suitable reducing agent such as Super Hydride® in a suitable solvent, preferably THF at a temperature of −20 to 50° C., preferably at around 25° C. to give compounds of formula 58-1. Amino alcohols of formula 58-1 are then treated with at least two equivalents of methanesulfonyl chloride and at least two equivalents of a suitable base, preferably pyridine in a suitable solvent, preferably pyridine at a temperature of −20 to 50° C. preferably around 25° C. to give intermediates of formula 58-2. Treatment of 58-2 with a strong base, preferably sec-butyllithium at a temperature of around −78° C. followed by warming to a temperature of around 25° C. affords intermediates of formula 58-3. Removal of the protecting group as described above, transforms 58-3 into 584.

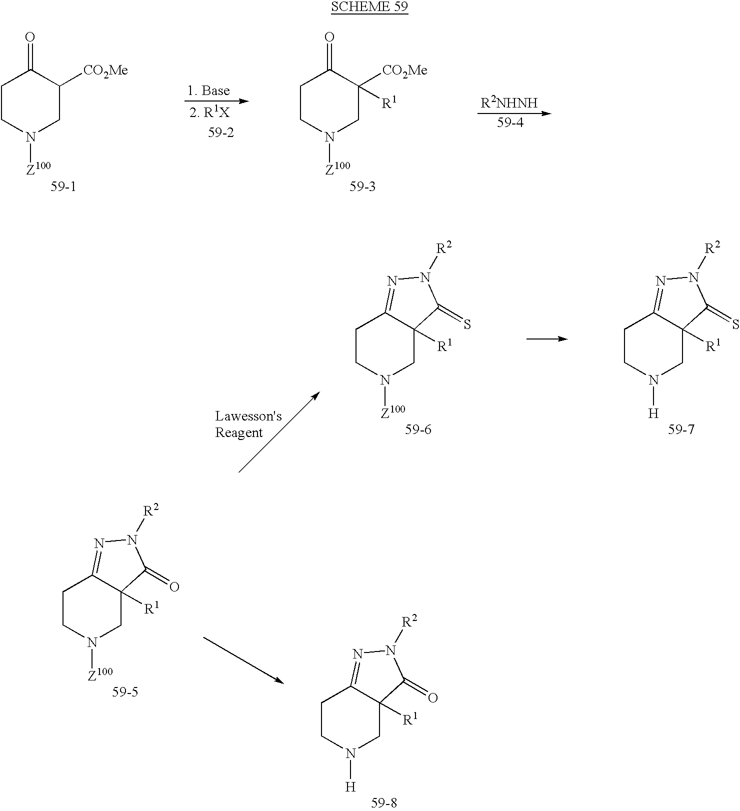

As illustrated in Scheme 59, treatment of an ester of formula 59-1 with a base such as sodium hydride in a solvent such as DMF followed by an alkyl halide 59-2 generates a compound of formula 59-3. Treating a compound of formula 59-3 with a hydrazine of formula 59-4 such as hydrazine or methyl-hydrazine in a solvent such as refluxing ethanol, followed by concentration and heating the residue in toluene at temperatures at or near reflux results in a compound of formula 59-5. Alternatively, 59-3 can be treated with a salt of a hydrazine in the presence of sodium acetate in refluxing ethanol to give 59-5. Deprotection of the amine generates a compound of formula 59-8. Thioamides of formula 59-6 can be formed by treating 59-5 with Lawesson's reagent in refluxing toluene or benzene. Removal of the protecting group transforms 59-6 into 59-7.

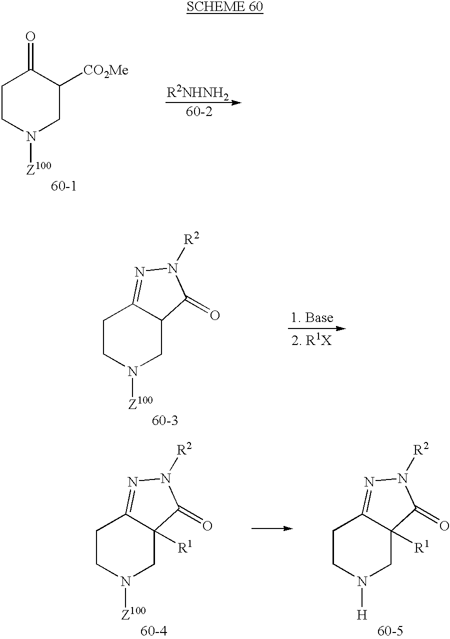

As illustrated in Scheme 60, treatment of a compound of formula 60-1 with a hydrazine of formula 60-2 in a solvent such as refluxing ethanol, followed by concentration and heating the residue in toluene at temperatures at or near reflux results in compounds of formula 60-3. Alternatively, 60-1 can be treated with a salt of a hydrazine in the presence of sodium acetate in refluxing ethanol to give 60-3. The amide of formula 60-3 can be treated with a base such as sodium hydride in a solvent such as DMF followed by an alkyl halide to give 604. Deprotection of the amine generates a compound of formula 60-5.

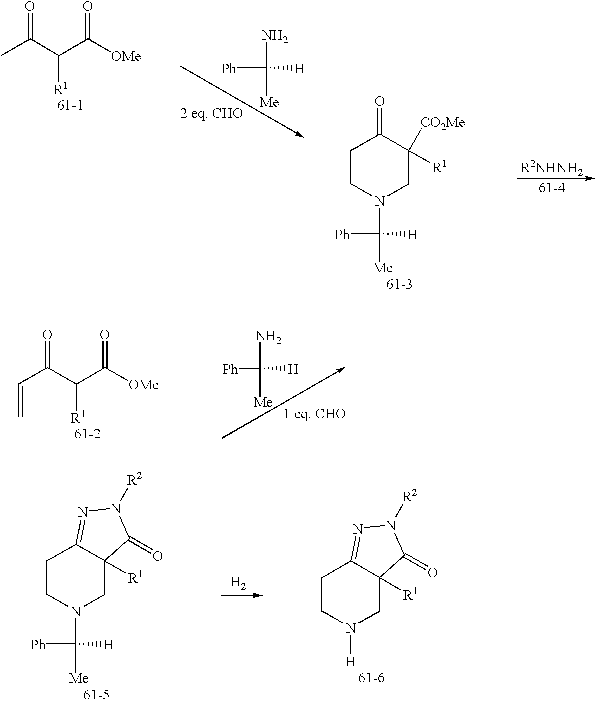

As illustrated in Scheme 61, reaction of a ketoester of formula 61-1 with a chiral amine such as alpha-methylbenzylamine with a suitable aldehyde such as formaldehyde, or reaction of a vinyl ketoester of formula 61-2 with a chiral amine such as alpha-methylbenzylamine with a suitable aldehyde such as formaldehyde, affords a compound of formula 61-3 via a double Mannich reaction. Reaction of 61-3 with a hydrazine generates a chiral compound of formula 61-5. Deprotection of the nitrogen with hydrogen and a suitable catalyst such as palladium affords compounds of formula 61-6.

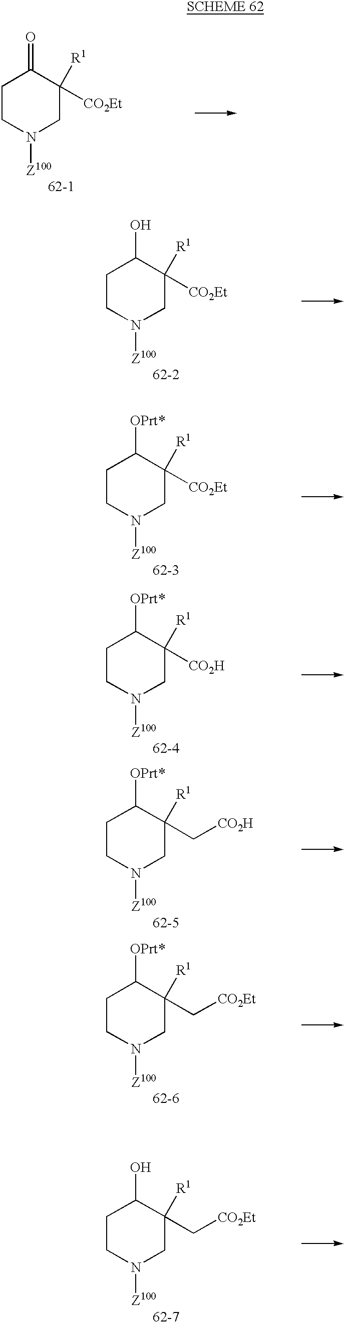

As illustrated in Scheme 62, treatment of a compound of formula 62-1 with a reducing agent such as sodium borohydride and protection of the nitrogen affords a compound of formula 62-2. Protection of the alcohol affords 62-3. Saponification of the ester affords a compound of formula 62-4. Reaction of 62-4 with thionyl chloride followed by treatment with diazomethane affords the homologated acid of formula 62-5. Esterification of 62-5 affords a compound of formula 62-6, which is 0-deprotected to give 62-7. Oxidation of 62-7 affords a ketone of formula 62-8. Reaction of 62-8 with a hydrazine, followed by nitrogen deprotection affords a compound of formula 62-9.

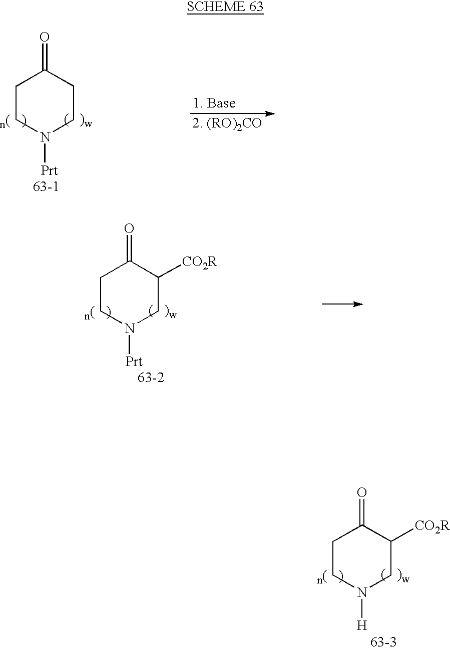

As illustrated in Scheme 63, treatment of a compound of formula 63-1 with a base such as sodium hydride in a solvent such as DMF followed by treatment with diethylcarbonate generates the ethyl ester of compound 63-2. Deprotection of the amine transforms 63-2 into 63-3.

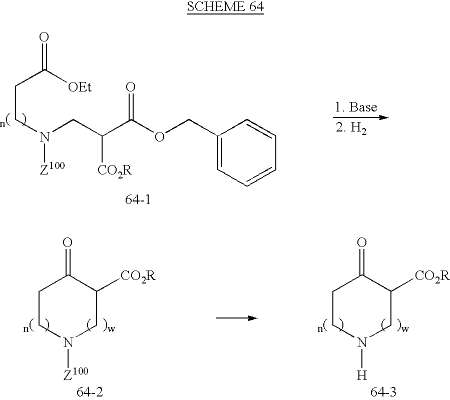

As illustrated in Scheme 64, treatment of a malonic ester of formula 64-1 with a base such as sodium hydride in a solvent such as DMF and subsequent hydrogenolysis of the benzyl group with hydrogen and a catalyst such as palladium in a suitable solvent such as methanol produces the ester of formula 64-2. Deprotection of the amine generates compounds of formula 64-3.

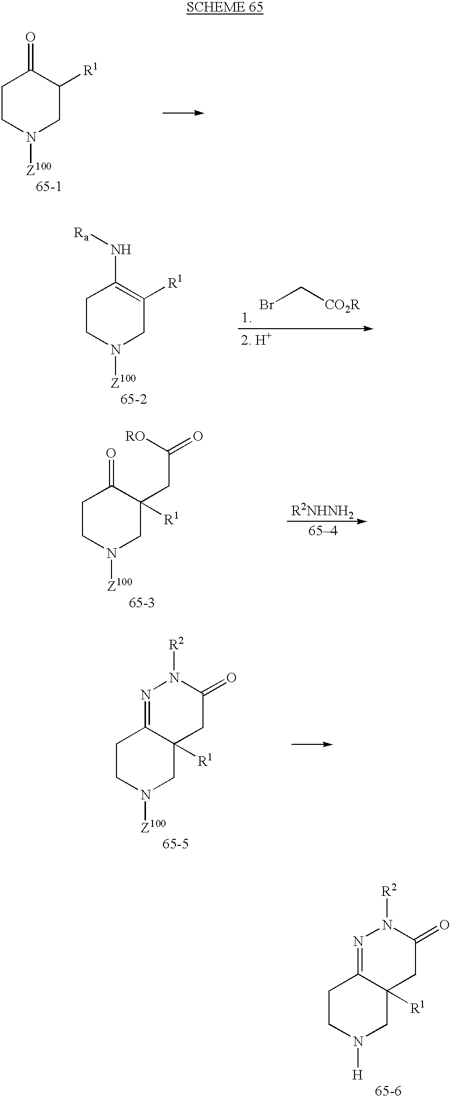

As illustrated in Scheme 65, treatment of a ketone of formula 65-1 with a secondary amine such as piperidine in a suitable solvent such as benzene with removal of water affords an enamine of formula 65-2. Alkylation of the enamine with an alpha-haloester such as ethylbromoacetate in a suitable solvent such as benzene or THF using a suitable base such as LDA or NaN(SiMe₃)₂ affords a ketoester of formula 65-3. Reaction with a hydrazine of formula 65-4 affords the compound of formula 65-5. Deprotection of the nitrogen affords compounds of formula 65-6.

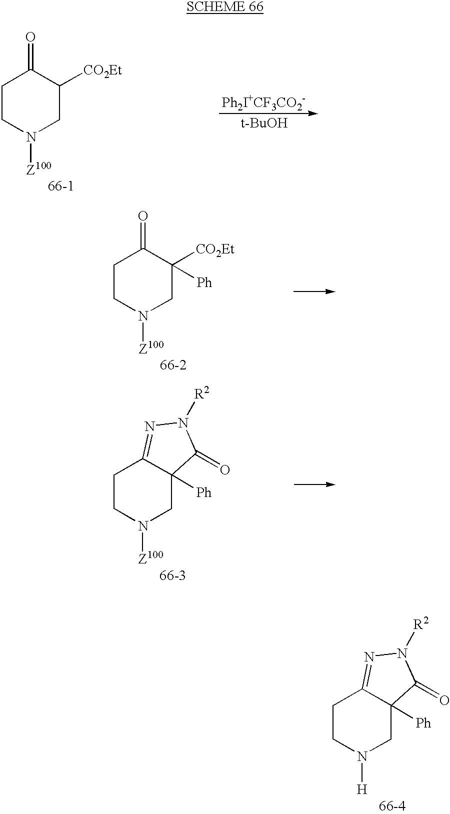

As illustrated in Scheme 66, treatment of a ketoester of formula 66-1 with an iodonium salt such as diphenyliodonium trifluoroacetate in a suitable solvent such as t-butanol generates a ketoester of formula 66-2. Reaction of 66-2 with a hydrazine generates a compound of formula 66-3. Deprotection of the nitrogen affords compounds of formula 66-4, see Synthesis, (9), 1984 p. 709 for a detailed description.

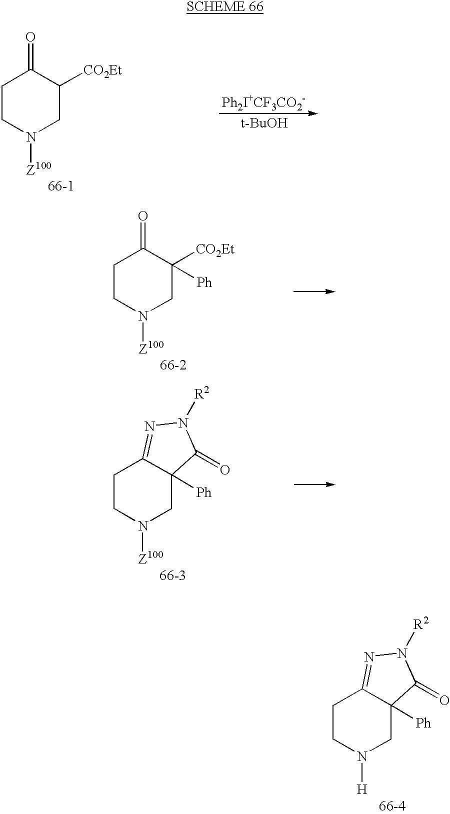

As illustrated in Scheme 66, treatment of a ketoester of formula 66-1 with an iodonium salt such as diphenyliodonium trifluoroacetate in a suitable solvent such as t-butanol generates a ketoester of formula 66-2. Reaction of 66-2 with a hydrazine generates a compound of formula 66-3. Deprotection of the nitrogen affords compounds of formula 66-4, see Synthesis, (9), 1984 p. 709 for a detailed description.

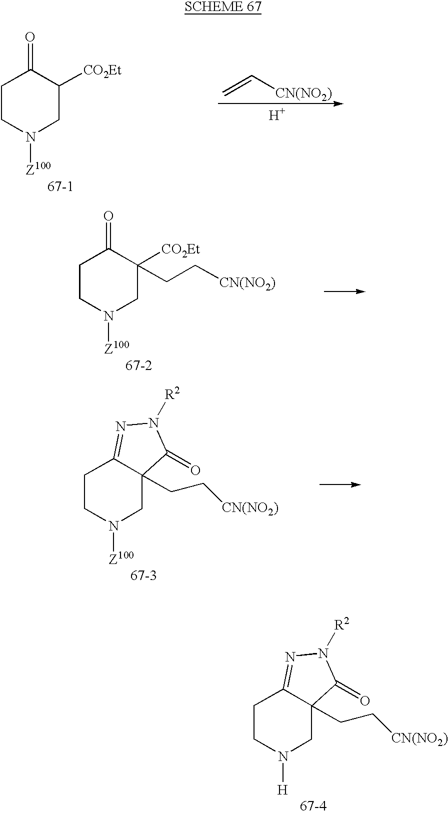

As illustrated in Scheme 67, treatment of a ketoester of formula 67-1 with an olefin such as acrylonitrile generates a ketoester of formula 67-2. Reaction of 67-2 with a hydrazine generates a compound of formula 67-3. Deprotection of the nitrogen affords compounds of formula 674.

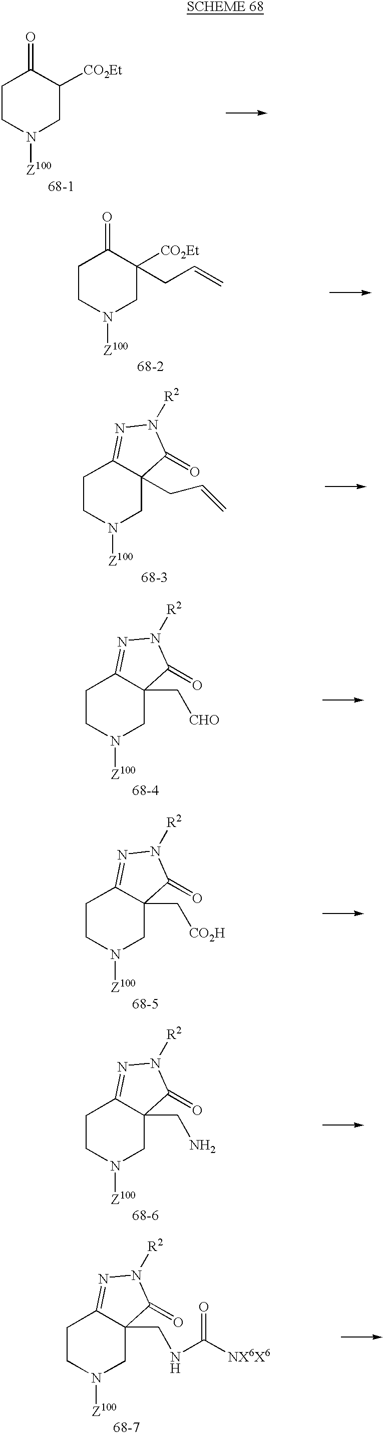

As illustrated in Scheme 68, treatment of a ketoester of formula 68-1 with allyl bromide and a suitable base such as sodium hydride in a suitable solvent such as DMF affords a ketoester of formula 68-2. Reaction of 68-2 with a hydrazine generates a compound of formula 68-3. Ozonolysis of 68-3 in a suitable solvent such as methylene chloride followed by treatment with a reducing agent such as dimethylsulfide affords an aldehyde of formula 68-4. Oxidation of 68-4 affords a carboxylic acid of formula 68-5. Curtius rearrangement of 68-5, followed by hydrolysis of the intermediate isocyanate affords a primary amine of formula 68-6. Treatment of a compound of formula 68-6 with an isocyanate or carbamate affords a urea of formula 68-7. Deprotection of the nitrogen affords compounds of formula 68-8.

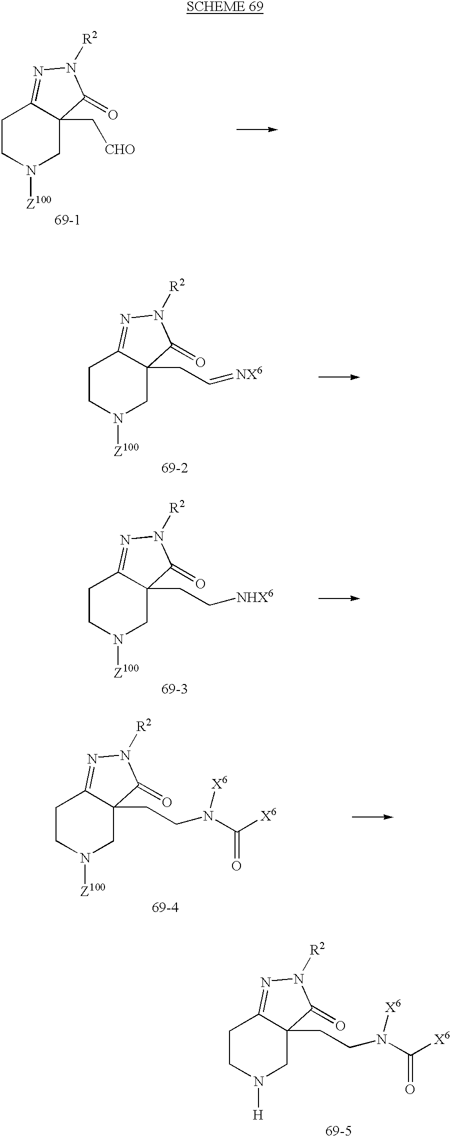

As illustrated in Scheme 69, treatment of a compound of formula 69-1 with a primary amine affords an imine of formula 69-2. Reduction of a compound of formula 69-2 affords a compound of formula 69-3. Treatment of a compound of formula 69-3 with an acylating agent affords a compound of formula 69-4. Deprotection of the nitrogen affords compounds of formula 69-5.

[chemical structure]

As illustrated in Scheme 70, treatment of a compound of formula 70-1 with a reducing agent such as sodium borohydride affords a compound of formula 70-2. Reaction of 70-2 with an acylating agent such as an isocyanate or carbamate affords compounds of formula 70-3. Deprotection of the nitrogen affords compounds of formula 70-4.

[chemical structure]

As illustrated in Scheme 71, treatment of a compound of formula 71-1 with a phosphine such as triphenyl phosphine and an azo compound such as diethylazodicarboxylate and an oxindole affords a compound of formula 71-2. Deprotection of the nitrogen affords the compound of formula 71-3.

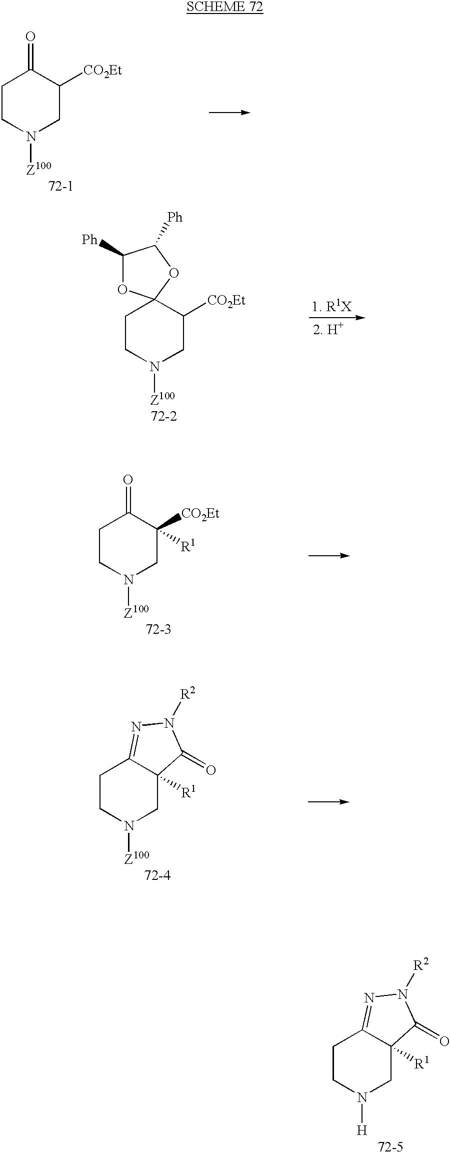

As illustrated in Scheme 72, treatment of a ketoester of formula 72-1 with a chiral diol and acid catalyst with removal of water in a suitable solvent such as benzene affords a chiral ketal of formula 72-2. Alkylation of 72-2 with an alkyl halide in the presence of a base such as LDA followed by acid-catalyzed hydrolysis of the ketal affords chiral ketoesters of formula 72-3. Reaction of 72-3 with a hydrazine generates chiral compounds of formula 72-4. Deprotection of the nitrogen affords compounds of formula 72-5.

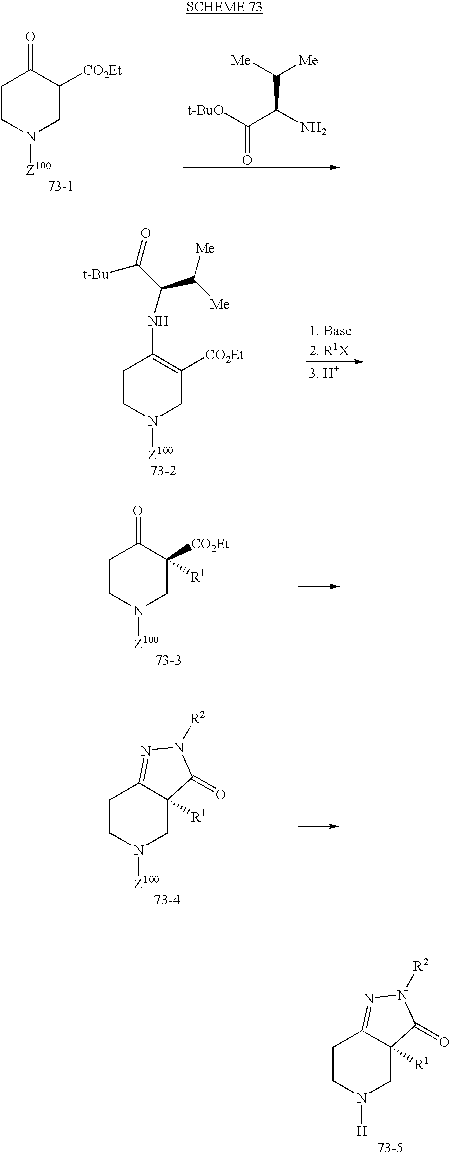

As illustrated in Scheme 73, treatment of a ketoester of formula 73-1 with a chiral amino acid ester such as valine t-butyl ester affords a chiral enamine of formula 73-2. Alkylation of 73-2 with an alkyl halide in the presence of a base such as LDA followed by acid-catalyzed hydrolysis of the enamine affords chiral ketoesters of formula 73-3. Reaction of 73-3 with a hydrazine generates chiral compounds of formula 73-4. Deprotection of the nitrogen affords compounds of formula 73-5.

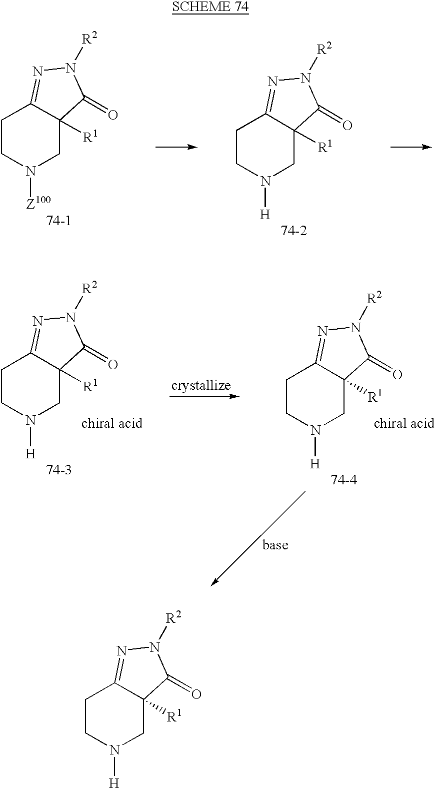

As illustrated in Scheme 21, deprotection of the nitrogen of 74-1 affords compounds of formula 74-2. Salt formation of 74-2 with a chiral acid affords a mixture of diastereomeric salts of formula 74-3. Crystallization of the diastereomeric salts affords the acid salt of chiral compounds of formula 74-4. Decomposition of the salt 74-4 with base liberates chiral compounds of formula 74-5.

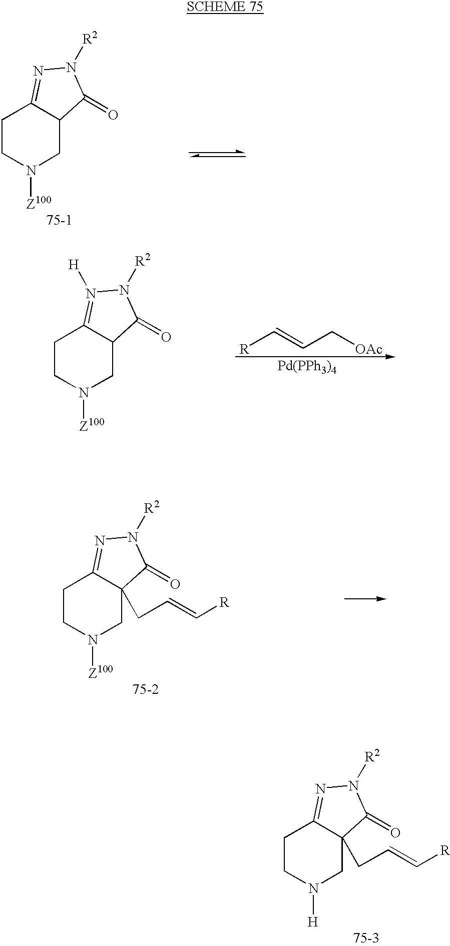

As illustrated in Scheme 75, alkylation of compounds of formula 75-1 with an allylic acetate in the presence of a suitable catalyst such as palladium tetrakis (triphenylphosphine) affords compounds of formula 75-2. Deprotection of the nitrogen affords compounds of formula 75-3, see Tetrahedron (50) p. 515, 1994 for a detailed discussion.

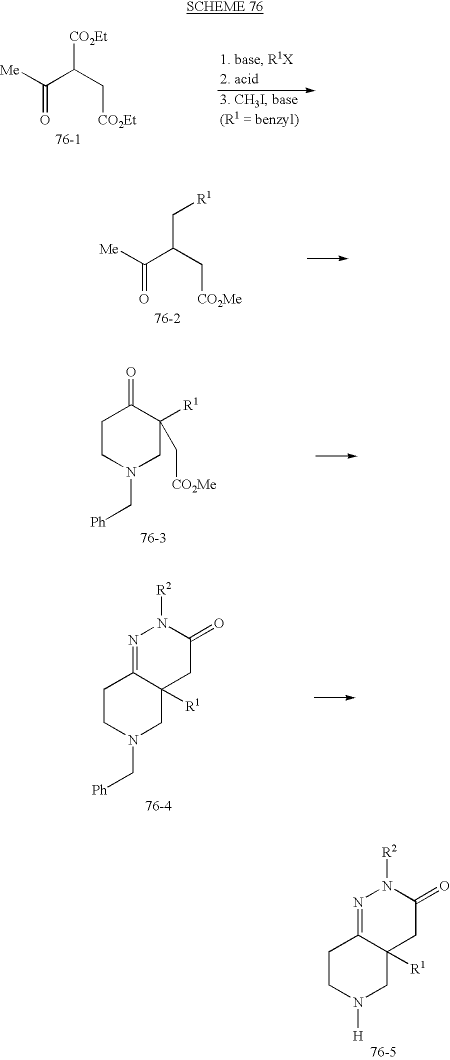

As illustrated in Scheme 76, treatment of a ketodiester of formula 76-1 with an alkyl halide in the presence of a base such as sodium hydride followed by acid-catalyzed hydrolysis and decarboxylation, followed by esterification with methyliodide and a suitable base affords a compound of formula 76-2. Reaction of a compound of formula 76-2 with a suitable aldehyde such as formaldehyde and benzylamine affords a compound of formula 76-3. Reaction of a compound of formula 76-3 with a hydrazine generates compounds of formula 76-4. Deprotection of the nitrogen affords compounds of formula 76-5.

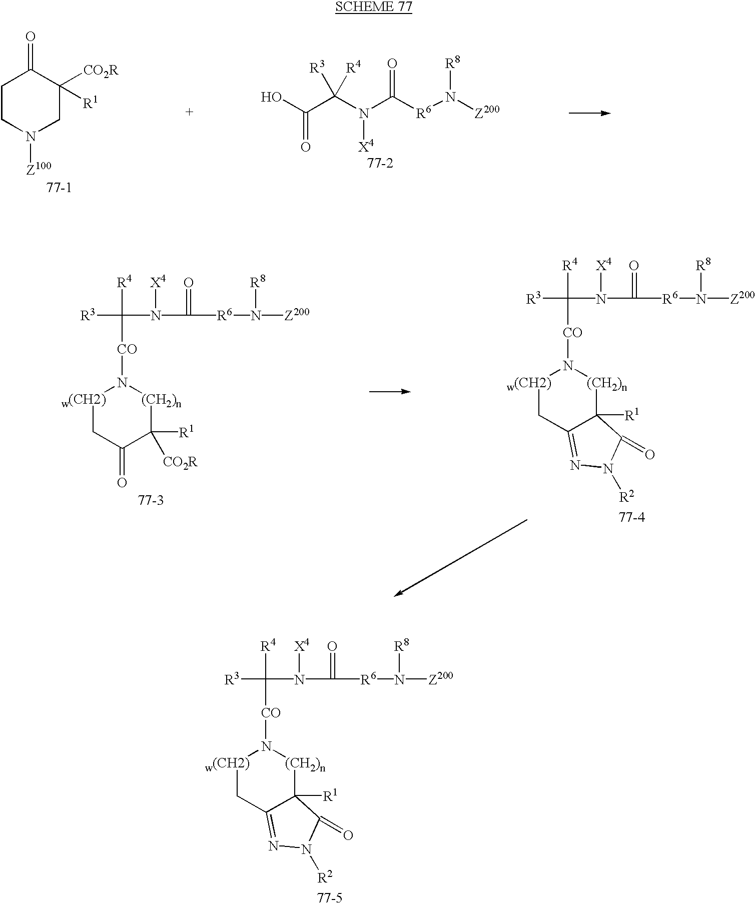

As illustrated in Scheme 77, treatment of an amine of formula 77-1 with an acid of formula 77-2 in an inert solvent such as dichloromethane or DMF by a coupling reagent such as EDC or DCC in the presence of HOBT affords compounds of formula 77-3. Reaction of compounds of formula 77-3 with a hydrazine generates compounds of formula 77-4. Deprotection of the nitrogen affords compounds of formula 77-5.

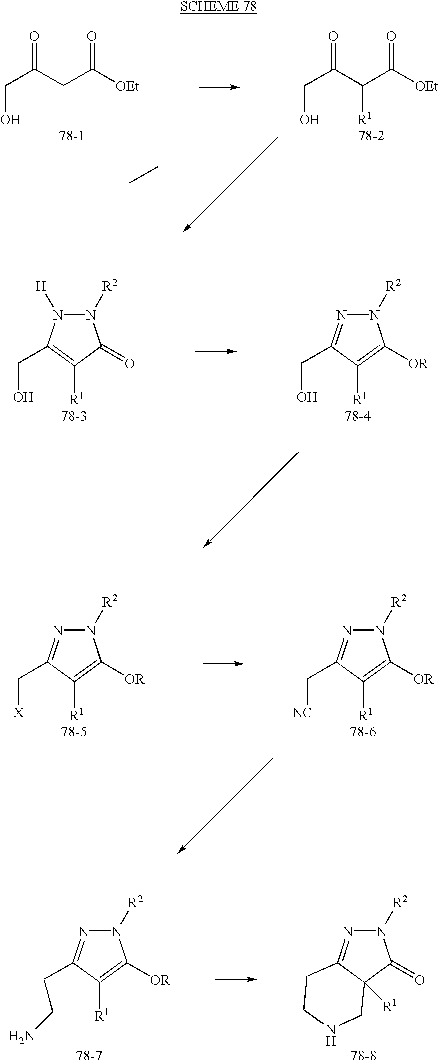

As illustrated in Scheme 78, treatment of a hydroxyacetoacetate ester of formula 78-1 with an alkyl halide in the presence of a suitable base such as sodium hydride affords compounds of formula 78-2. Reaction of 78-2 with a hydrazine generates compounds of formula 78-3. 0-Alkylation of the carbonyl oxygen of 78-3 affords 78-4 which is converted to the halide 78-5. Displacement of the halide X by cyanide ion affords the nitrile 78-6. Reduction of 78-6 gives the primary amine 78-7 which is deprotected and cyclized in the presence of formaldehyde to afford 78-8

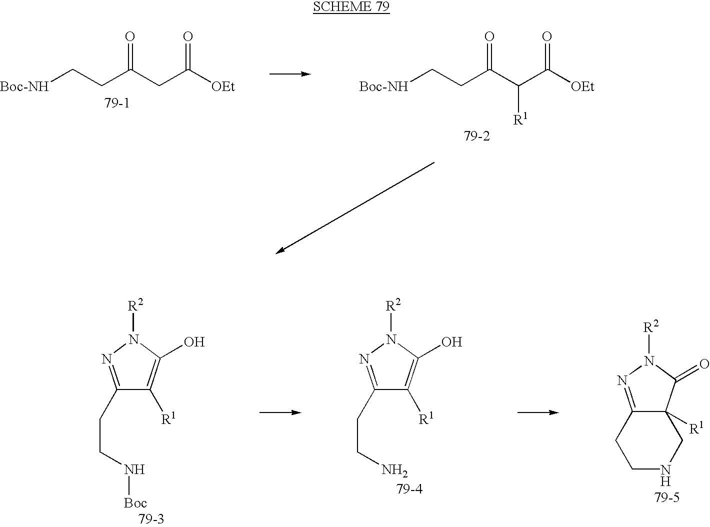

As illustrated in Scheme 79, treatment of a beta-keto-protected aminovalerate such as 79-1 with an alkyl halide in the presence of a suitable base such as sodium hydride affords compounds of formula 79-2. Reaction of compounds of formula 79-2 with a hydrazine generates compounds of formula 79-3. Deprotection of compounds of formula 99 affords primary amines of formula 79-4. Cyclization of compounds of formula 79-4 in the presence of formaldehyde affords compounds of formula 79-5.

[chemical structure]

As illustrated in Scheme 80, treatment of the amine of formula 80-1 with an acid such as 80-2 in the presence of EDC and HOAT in a suitable solvent provides keto-esters of formula 80-3. The keto-ester 80-3 can be treated with a salt of hydrazine in the presence of sodium acetate in refluxing ethanol to give hydrazines of formula 80-4. Deprotection under suitable conditions gives amines of formula 80-5. Coupling of intermediates of formula 80-5 to amino acids of formula 80-6 can be effected as described above to give intermediates of formula 80-7. Deprotection of amine 80-7 affords compounds of formula 80-8.

In the above structural formulae and throughout the instant application, the following terms have the indicated meanings unless expressly stated otherwise:

The alkyl groups are intended to include those alkyl groups of the designated length in either a straight or branched configuration which may optionally contain double or triple bonds. Exemplary of such alkyl groups are methyl, ethyl, propyl, isopropyl, butyl, sec-butyl, tertiary butyl, pentyl, isopentyl, hexyl, isohexyl, allyl, ethynyl, propenyl, butadienyl, hexenyl and the like.

When the definition C₀-alkyl occurs in the definition, it means a single covalent bond.

The alkoxy groups specified above are intended to include those alkoxy groups of the designated length in either a straight or branched configuration which may optionally contain double or triple bonds. Exemplary of such alkoxy groups are methoxy, ethoxy, propoxy, isopropoxy, butoxy, isobutoxy, tertiary butoxy, pentoxy, isopentoxy, hexoxy, isohexoxy, allyloxy, 2-propynyloxy, isobutenyloxy, hexenyloxy and the like.

The term “halogen” or “halo” is intended to include the halogen atoms fluorine, chlorine, bromine and iodine.

The term “halogenated alkyl” is intended to include an alkyl group as defined hereinabove substituted by one or more halogen atoms as defined hereinabove.

The term “halogenated cycloalkyl” is intended to include a cycloalkyl group substituted by one or more halogen atoms as defined hereinabove.

The term “aryl” is intended to include phenyl and naphthyl. The term “heteroaryl” is intended to include aromatic 5- and 6-membered rings with 1 to 4 heteroatoms or fused 5- and/or 6-membered bicyclic rings with 1 to 4 heteroatoms of nitrogen, sulfur or oxygen. Examples of such heterocyclic aromatic rings are pyridine, thiophene (also known as thienyl), furan, benzothiophene, tetrazole, indole, N-methylindole, dihydroindole, indazole, N-formylindole, benzimidazole, thiazole, pyrimidine, pyrrole, imidazole, oxazole, thiazole, pyrazole, purine, quinoline, isoquinoline, pyrazine, pyrimidine, triazine, pyridazine and thiodiazole.

The expression “prodrug” refers to compounds that are drug precursors which following administration, release the drug in vivo via some chemical or physiological process (e.g., a prodrug on being brought to the physiological pH is converted to the desired drug form). Exemplary prodrugs upon cleavage release the corresponding free acid, and such hydrolyzable ester-forming residues of the compounds of this invention include but are not limited to carboxylic acid substituents (e.g., when R¹ is -(CH₂)_{q}C(O)OX⁶ where X⁶ is hydrogen, or when R² or A¹ contains carboxylic acid) wherein the free hydrogen is replaced by (C₁-C₄)alkyl, (C₂-C₁₂)alkanoyloxymethyl, (C₄-Cg)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl) aminomethyl having from 3 to 9 carbon atoms, 1-(N-(alkoxycarbonyl)amino) ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂) alkyl and piperidino-, pyrrolidino- or morpholino(C₂-C₃)alkyl.

Other exemplary prodrugs release an alcohol of Formula I wherein the free hydrogen of the hydroxyl substituent (e.g., when R¹ contains hydroxyl) is replaced by (C₁-C₆)alkanoyloxymethyl, 1 -((C₁-C₆)alkanoyloxy)ethyl, 1-methyl-1 -((C₁-C₆)alka-noyloxy)ethyl, (C₁-C₆)alkoxycarbonyloxymethyl, N-(C₁-C₆)alkoxy-carbonylamino-methyl, succinoyl, (C₁-C₆)alkanoyl, α-amino(C₁-C₄)alkanoyl, arylacetyl and α-aminoacyl, or α-aminoacyl-α-aminoacyl wherein said α-aminoacyl moieties are independently any of the naturally occurring L-amino acids found in proteins, —P(O)(OH)₂, —P(O)(O(C₁-C₆)alkyl)₂ or glycosyl (the radical resulting from detachment of the hydroxyl of the hemiacetal of a carbohydrate).

Prodrugs of this invention where a carboxyl group in a carboxylic acid of Formula I is replaced by an ester may be prepared by combining the carboxylic acid with the appropriate alkyl halide in the presence of a base such as potassium carbonate in an inert solvent such as DMF at a temperature of about 0° C. to 100° C. for about 1 to about 24 hours. Alternatively, the acid is combined with the appropriate alcohol as solvent in the presence of a catalytic amount of acid such as concentrated sulfuric acid at a temperature of about 20° C. to 120° C., preferably at reflux, for about 1 hour to about 24 hours. Another method is the reaction of the acid in an inert solvent such as THF, with concomitant removal of the water being produced by physical (e.g., Dean Stark trap) or chemical (e.g., molecular sieves) means.

Prodrugs of this invention where an alcohol function has been derivatized as an ether may be prepared by combining the alcohol with the appropriate alkyl bromide or iodide in the presence of a base such as potassium carbonate in an inert solvent such as DMF at a temperature of about 0° C. to 100° C. for about 1 to about 24 hours. Alkanoylaminomethyl ethers may be obtained by reaction of the alcohol with a bis-(alkanoylamino)methane in the presence of a catalytic amount of acid in an inert solvent such as THF, according to a method described in U.S. Pat. No. 4,997,984. Alternatively, these compounds may be prepared by the methods described by Hoffman et al. in J. Org. Chem. 1994, 59, p. 3530.

Many protected amino acid derivatives are commercially available, where the protecting groups, Prt, Prt′ or Prt″, are, for example, BOC, CBZ, FMOC, benzyl or ethoxycarbonyl groups. Other protected amino acid derivatives can be prepared by literature methods well-known to one skilled in the art. Some substituted piperazines and piperidines are commercially available, and many other piperazines and 4-substituted piperidines are known in the literature. Various heterocyclic substituted piperidines and piperazines can be prepared following literature methods using derivatized heterocyclic intermediates. Alternatively, the heterocyclic rings of such compounds can be derivatized by standard means, such as coupling with CDI, hydrogenation of aromatic heterocycles, etc. as is well-known to those skilled in the art.

Certain of the above defined terms may occur more than once in the above formula and upon such occurrence each term shall be defined independently of the other.

The compounds of the instant invention all have at least one asymmetric center as noted by the asterisk in the structural Formula I. Additional asymmetric centers may be present on the molecule depending upon the nature of the various substituents on the molecule. Each such asymmetric center will produce two optical isomers and it is intended that all such optical isomers, as separated, pure or partially purified optical isomers, racemic mixtures or diastereomeric mixtures thereof, be included within the scope of the instant invention.

The instant compounds are generally isolated in the form of their pharmaceutically acceptable acid addition salts, such as the salts derived from using inorganic and organic acids. Examples of such acids are hydrochloric, nitric, sulfuric, phosphoric, formic, acetic, trifluoroacetic, propionic, maleic, succinic, D-tartaric, L-tartaric, malonic, methane sulfonic and the like. In addition, certain compounds containing an acidic function such as a carboxy can be isolated in the form of their inorganic salt in which the counter-ion can be selected from sodium, potassium, lithium, calcium, magnesium and the like, as well as from organic bases.

The pharmaceutically acceptable salts are formed by taking about 1 equivalent of a compound of Formula I and contacting it with about 1 equivalent of the appropriate corresponding acid of the salt which is desired. Work-up and isolation of the resulting salt is well-known to those of ordinary skill in the art.

It will be recognized that the compounds of Formula I of this invention can exist in radiolabelled form, i.e., said compounds may contain one or more atoms containing an atomic mass or mass number different from the atomic mass or mass number ordinarily found in nature. Radioisotopes of hydrogen, carbon, phosphorous, fluorine and chlorine include ³H, ¹⁴C, ³²p, 35S, ¹⁸F and ³⁶Cl, respectively. Compounds of Formula I of this invention which contain those radioisotopes and/or other radioisotopes of other atoms are within the scope of this invention. Tritiated, i.e., ³H, and carbon-14, i.e., ¹⁴C, radioisotopes are particularly preferred for their ease of preparation and detectability. Radiolabelled compounds of Formula I of this invention can generally be prepared of methods well known to those skilled in the art. Conveniently, such radiolabelled compounds can be prepared by carrying out the procedures disclosed in the above Schemes and/or in the Examples and Preparations below by substituting a readily available radiolabelled reagent for a non-radiolabelled reagent.

Biological Assays

A. MCR-4 Binding Assay

To prepare membranes for the MCR-4 binding assay, human embryonic kidney cells (HEK 293) that express human MCR-4 (obtained from University of Michigan School of Medicine) are grown in suspension culture in Dulbecco's Modified Eagles Medium (Gibco-BRL, #111995-065) containing 10% fetal bovine serum (certified, Gibco-BRL), penicillin G (10 units/ml), streptomycin sulfate (10 microgram/ml), and 0.6 g/l geneticin (Gibco-BRL). The cells are then separated from the culture medium by centrifugation at 1000×g for 10 minutes at 4° C. and resuspended in phosphate-buffered saline. The cells are then centrifuged at 1000×g for 10 minutes at 40C and then resuspended in ice cold Homogenization Buffer (HB=10 mM HEPES, pH 7.5, 1 mM EDTA, 1 mM EGTA and a 1:1000 dilution of protease inhibitors: Sigma # P-8340). The cells are then allowed to incubate on ice for 10 minutes, followed by homogenization on ice with 20 strokes of a Dounce homogenizer. The lysate is then centrifuged at 1000×g for 10 minutes at 4° C. The supernatant is transferred into new centrifuge tubes and pellet. is discarded. The supernatant is then centrifuged at 25,000×g for 25 minutes at 4° C. The supernatant is discarded and the cell pellet (containing plasma membrane) is resuspended in ice-cold HB, and subjected to two complete resuspension/centrifugation cycles. The final pellet is resuspended in HB at a membrane protein concentration between 1-5 mg/ml and aliquots are frozen at -70° C. for long term storage.

To measure the binding affinity of test agents at human MCR-4, 50 μl of binding buffer (BB=25 mM HEPES, pH 7.5, 1.5 mM CaCI, 1 mM MgSO4, 100 mM NaCl, 0.2% BSA, and protease inhibitors: Sigma catalogue #P-8340) is added into each well of a 96 well polypropylene plate (300ul Falcon). 50 μl of test agent is added in triplicate to the appropriate wells. Next 100 μl of ¹²⁵I-NDP-MSH (New England Nuclear, catalogue NEX 372) is added to a final concentration in each well of 50 pM, followed by 50 μl of MCR-4 membranes (0.5 ug of membrane protein/well). The plates are placed on a plate shaker (Lab line Instruments, Inc.) in an incubator at 37° C. The binding reaction is allowed to proceed for 1 hour. The plates are then removed from the shaker, and placed in a Packard harvester and the binding assay is aspirated onto Millipore 96 Well GF/C Filterplates (pre-soaked in a 0.5 % polyethylenimine/H₂O solution). The plate is then washed twice with 300 μI of ice cold wash buffer (25 mM HEPES, pH 7.5, 1.5 mM CaCl, 1 mM MgSO4, 100 mM NaCl). The filterplate is then dried for 20 minutes in a 42° C. oven. 30 μl of Wallac Supermix scintillation fluid is added to all wells. The radioactivity on each plate is measured using a Wallac Microbeta 96-well plate scintillation counter. The IC₅₀ for each compound is than determined by non-linear regression analysis using a software package (Prism by Graphpad).

Functional Assay: Functional cell based assays are developed to discriminate agonists and antagonists

Agonism: The functional (agonist) activity of test agents at MCR-4 is determined by measuring cAMP levels in CHO cells that have been engineered to express human MCR-4. CHO/MCR-4 cells are plated into 96-well plates (plating density=14,000 cells/well in DMEM/F12 medium (Gibco-BRL) containing 10% fetal bovine serum (Gibco-BRL), penicillin G (10 units/ml), streptomycin sulfate (10 microgram/ml) and geneticin (G418) at 400 microgram/ml). 24 hours after plating, the culture medium is changed to serum-free medium. 18 hours later, the functional assay is initiated by adding test agent from a DMSO stock (final DMSO concentration=0.5%) to the cells. Plates are incubated for 50 min at 37° C. The assay is terminated by aspiration of the medium, addition of 100 ul of 0.01 N HCl followed by incubation at room temperature for 20 minutes on a rotating platform. Each well is then neutralized by addition of 6 ul of 0.2N NaOH, and the plates are frozen plate at -20° C. Plates are then thawed and the cAMP concentration in the lysate is determined using the cAMP [¹²⁵I] Flashplate Assay (New England Nuclear) and a Wallac Microbeta 96-well plate scintillation counter. The level of cAMP in reponse to a test agent is calculated first as pmol/ml, corrected for basal cAMP, then expressed as a percentage of maximal alphaMSH (defined as the cAMP response to 1 uM alphaMSH). EC50s for test agents are then determined by non-linear regression analysis using the software package Prism by Graphpad.

Antagonism: To measure antagonism of an unknown compound, the above assay is followed except a 1 to 1000 nM alpha-MSH agonist challenge is added to the wells with the unknown compound. The level of cAMP is expressed as a percentage of the challenge alpha-MSH (1 to 1000 nM). IC50 for test compounds are determined by non-linear regression analysis using the software package Prism by Graphpad.

In Vivo Food Intake Models Induced food intake model: Wistar rats are fasted overnight and injected with a test compound intracerebroventicularly (2-6ul in 5 -10% DMSO), intraperitoneally, sub-cutaneously or oral gavage. Food intake is determined in home cages or using a computerized system (The computer system measures food changes through a balance system). Cumulative food intake and food intake intervals are taken 1, 2, 4, and 8 hour time points in home cages and in 5 minute intervals over 24 hours in the computerized system. Biochemicals parameters relating to obesity, including leptin, insulin, serum glucose, triglyceride, free fatty acid and cholesterol levels are determined.

24 hour food intake model: Free fed Wistar rats are injected with a test compound intracerebroventicularly (2-6ul in 5 -10% DMSO), intraperitoneally, sub-cutaneously or oral gavage, then placed in a computerized food intake system. Cumulative food intake and food intake intervals are in 5 minute intervals over the next 24 hours in the computerized system. Biochemicals parameters relating to obesity, including leptin, insulin, serum glucose, triglyceride, free fatty acid and cholesterol levels are determined.

In Vivo Thermogenesis Models

Whole body oxygen consumption is measured using an indirect calorimeter (Oxymax from Columbus Instruments, Columbus, Ohio) in Sprague Dawley rats. The rats (300-380 g body weight) are placed in calorimeter chambers and the chambers are placed in activity monitors. Basal pre-dose oxygen consumption and ambulatory activity are measured every 10 minutes for 2.5 to 3 hours. At the end of the basal pre-dosing period, the chambers are opened and the animals are administered a single dose of compound (the usual dose range is 0.001 to 100 mg/kg) by oral gavage (or other route of administration as specified, i.e. s.c., i.p., i.v., i.c.v.). Drugs are prepared in methylcellulose, water or other specified vehicle (examples include PEG400, propylene glycol or DMSO). Oxygen consumption and ambulatory activity are measured every 10 minutes for an additional 1-6 hours post-dosing.

The Oxymax calorimeter software calculates the oxygen consumption (ml/kg/h) based on the flow rate of air through the chambers and difference in oxygen content at inlet and output ports. The activity monitors have 15 infrared light beams spaced one inch apart on each axis, ambulatory activity is recorded when two consecutive beams are broken and the results are recorded as counts. Resting oxygen consumption, during pre- and post-dosing, is calculated by averaging the 10-min O2 consumption values, excluding periods of high ambulatory activity (ambulatory activity count>100) and excluding the first 5 values of the pre-dose period and the first value from the post-dose period. Change in oxygen consumption is reported as percent and is calculated by dividing the post-dosing resting oxygen consumption by the pre-dose oxygen consumption ×100.

Experiments will typically be done with n=4 rats and results reported are mean +/−SEM.

B. Rat Ex Copula Assay

Sexually mature male Caesarian Derived Sprague Dawley (CD) rats (over 60 days old) are used with the suspensory ligament surgically removed to prevent retraction of the penis back into the penile sheath during the ex copula evaluations. Animals receive food and water ad lib and are kept on a normal light/dark cycle. Studies are conducted during the light cycle.

a) Conditioning to Supine Restraint for Ex Copula Reflex Tests.

This conditioning takes ˜4 days. Day 1, the animals are placed in a darkened restrainer and left for 15 -30 minutes. Day 2, the animals are restrained in a supine position in the restrainer for 15 -30 minutes. Day 3, the animals are restrained in the supine position with the penile sheath retracted for 15 -30 minutes. Day 4, the animals are restrained in the supine position with the penile sheath retracted until penile responses are observed. Some animals require additional days of conditioning before they are completely acclimated to the procedures; non-responders are removed from further evaluation. After any handling or evaluation animals are given a treat to ensure positive reinforcement.

b) Ex Copula Reflex Tests.

Rats are generally restrained in a supine position with their anterior torso placed inside a cylinder of adequate size to allow for normal head and paw grooming. For a 400 -500 gram rat, the diameter of the cylinder is approximately 8 cm. The lower torso and hind limbs are restrained with a non-adhesive material (vetrap). An additional piece of vetrap with a hole in it, through which the glans penis will be passed, is fastened over the animal to maintain the preputial sheath in a retracted position. Penile responses will be observed, typically termed ex copula genital reflex tests. Typically, a series of penile erections will occur spontaneously within a few minutes after sheath retraction. The types of normal reflexogenic erectilve responses include elongation, engorgement, cup and flip. An elongation is classified as an extension of the penile body. Engorgement is a dilation of the glans penis. A cup is defined as an intense erection where the distal margin of the glans penis momentarily flares open to form a cup. A flip is dorsiflexion of the penile body.

Baseline and or vehicle evaluations are conducted to determine how and if an animal will respond. Some animals have a long duration until the first response while others are non-responders altogether. During this baseline evaluation latency to first response, number and type of responses are recorded. The testing time frame is 15 minutes after the first response.

After a minimum of 1 day between evaluations, these same animals are administered the test compound at 20 mg/kg and evaluated for penile reflexes. All evaluations are videotaped and scored later. Data are collected and analyzed using paired 2 tailed t-tests to compared baseline and/ or vehicle evaluations to drug treated evaluations for individual animals. Groups of a minimum of 4 animals are utilized to reduce variability.

Positive reference controls are included in each study to assure the validity of the study. Animals can be dosed by a number of routes of administration depending on the nature of the study to be performed. The routes of administration includes intravenous (IV), intraperitoneal (IP), subcutaneous (SC) and intracerebral ventricular (ICV).

C. Models of Female Sexual Dysfunction

Rodent assays relevant to female sexual receptivity include the behavioral model of lordosis and direct observations of copulatory activity. There is also a urethrogenital reflex model in anesthetized spinally transected rats for measuring orgasm in both male and female rats. These and other established animal models of female sexual dysfunction are described in McKenna, K. E. et al., A Model For The Study Of Sexual Function In Anesthetized Male And Female Rats, Am. J. Physiol. (Regulatory Integrative Comp. Physiol 30): R1276 -R1285, 1991; McKenna, K. E., et al., Modulation By Peripheral Serotonin Of The Threshold For Sexual Reflexes In Female Rats, Pharm. Bioch. Behav., 40:151-156, 1991; and Takahashi, L. K., et al., Dual Estradiol Action In The Diencephalon And The Regulation Of Sociosexual Behavior In Female Golden Hamsters, Brain Res., 359: 194 -207, 1985.

Utility

Compounds of formula I are melanocortin receptor agonists and as such are useful in the treatment, control or prevention of diseases, disorders or conditions responsive to the activation of one or more of the melanocortin receptors including, but are not limited to, MC-1, MC-2, MC-3, MC-4, or MC-5. Such diseases, disorders or conditions include, but are not limited to, obesity (by reducing appetite, increasing metabolic rate, reducing fat intake or reducing carbohydrate craving, diabetes mellitus (by enhancing glucose tolerance, decreasing insulin resistance), hypertension, hyperlipidemia, osteoarthritis, cancer, gall bladder disease, sleep apnea, depression, anxiety, compulsion, neuroses, insomnia/sleep disorder, substance abuse, pain, male and female sexual dysfunction (including impotence, loss of libido and erectile dysfunction), fever, inflammation, immune modulation, rheumatoid arthritis, skin tanning, acne and other skin disorders, neuroprotective and cognitive and memory enhancement including the treatment of Alzheimer's disease. Some compounds of formula I show highly specific activity toward the melanocortin4 receptor which makes them especially useful in the prevention and treatment of obesity, as well as male and female sexual dysfunction.

Administration and Dose Ranges

Any suitable route of administration may be employed for providing a mammal, especially a human with an effective dosage of a compound of the present invention. For example, oral, rectal, topical, parental, ocular, pulmonary, nasal, and the like may be employed. Dosage forms include tablets, troches, dispersions, suspensions, solutions, capsules, creams, ointments, aerosols, and the like. Preferably compounds of formula I are administered orally.

The effective dosage of active ingredient employed may vary depending on the particular compound employed, the mode of administration, the condition being treated and the severity of the condition being treated. Such dosage may be ascertained readily by a person skilled in the art.

When treating obesity, in conjunction with diabetes and/or hyperglycemia, or alone, generally satisfactory results are obtained when the compounds of the present invention are administered at a daily dosage of from 0.01 milligram to about 100 milligrams per kilogram of animal body weight, preferably given in a single dose or in divided doses two to six times a day, or in sustained release form. In the case of a 70 kg adult human, the total daily dose will generally be from about 0.7 milligrams to about 3500 milligrams. This dosage regimen may be adjusted to provide the optimal therapeutic response.

When treating diabetes mellitus and/or hyperglycemia, as well as other diseases or disorders for which compounds of formula I are useful, generally satisfactory results are obtained when the compounds of the present invention are administered at a daily dosage of from about 0.001 milligram to about 100 milligram per kilogram of animal body weight, preferably given in a single dose or in divided doses two to six times a day, or in sustained release form. In the case of a 70 kg adult human, the total daily dose will generally be from about 0.07 milligrams to about 350 milligrams. This dosage regimen may be adjusted to provide the optimal therapeutic response.

For the treatment of sexual dysfunction compounds of the present invention are given in a dose range of 0.001 milligram to about 100 milligram per kilogram of body weight, preferably as a singe dose orally or as a nasal spray.

Pharmaceutical Compositions

Another aspect of the present invention provides pharmaceutical compositions which comprises a compound of formula I and a pharmaceutically acceptable carrier. The pharmaceutical compositions of the present invention comprise a compound of formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (opthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media my be employed, such as, for example, water, glycols, oils, alcohols, flavoring agents, preservatives, coloring agents and the like in the case of oral liquid preparations such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol, (e.g. glycerol, propylene glycol and liquid polyethylene glycol), suitable mixtures thereof, and vegetable oils.

Combination Therapy

Compounds of formula I may be used in combination with other drugs that are used in the treatment/prevention/suppression or amelioration of the diseases or conditions for which compounds of formula I are useful. Such other drugs may be administered, by a route and in an amount commonly used therefor, contemporaneously or sequentially with a compound of formula I. When a compound of formula I is used contemporaneously with one or more other drugs, a pharmaceutical composition containing such other drugs in addition to the compound of formula I is preferred. Accordingly, the pharmaceutical compositions of the present invention include those that also contain one or more other active ingredients in addition to a compound of formula I. Examples of other active ingredients that may be combined with a compound of formula I, either administered separately or in the same pharmaceutical compositions, include, but are not limited to:

(a) insulin sensitizers including (i) PPARγ agonists such as the glitazones (e.g. troglitazone, pioglitazone, englitazone, MCC-555, BRL49653 and the like), and compounds disclosed in WO97/27857, 97/28115, 97/282137 and 97/27847; (ii) biguanides such as metformin and phenformin;

(b) insulin or insulin mimetics;

(c) sulfonylureas such as tolbutamide and glipizide;

(d) α-glucosidase inhibitors (such as acarbose),

(e) cholesterol lowering agents such as (i) HMG-CoA reductase inhibitors (lovastatin, simvastatin and pravastatin, fluvastatin, atorvastatin, and other statins), (ii) sequestrants (cholestyramine, colestipol and dialkylaminoalkyl derivatives of a cross-linked dextran), (ii) nicotinyl alcohol nicotinic acid or a salt thereof, (iii) proliferator-activater receptor α agonists such as fenofibric acid derivatives (gemfibrozil, clofibrat, fenofibrate and benzafibrate), (iv) inhibitors of cholesterol absorption for example beta-sitosterol and (acyl CoA:cholesterol acyltransferase) inhibitors for example melinamide, (v) probucol, (vi) vitamin E, and (vii) thyromimetics;

(f) PPARδ agonists such as those disclosed in WO97/28149;

(g) antiobesity compounds such as fenfluramine, dexfenfluramine, phentermine, sibutramine, orlistat, or β₃ adrenergic receptor agonists;

(h) feeding behavior modifying agents such as neuropeptide Y antagonists (e.g. neuropeptide Y5) such as those disclosed in WO 97/19682, WO 97/20820, WO 97/20821, WO 97/20822 and WO 97/20823;

(i) PPARα agonists such as described in WO 97/36579 by Glaxo;

(j) PPARγ antagonists as described in WO 97/10813;

(k) serotinin reuptake inhibitors such as fluoxetine and sertraline;

(I) growth hormone secretagogues such as MK-0677; and

(m) agents useful in the treatment of male and/or female sexual dysfunction such as phosphodiester V inhibitors such as \grave{s}ildenafil, and α-2 adrenergic receptor antagonists.

### Example 1

### 1,2,3,4-Tetrahydro-isoquinoline-(S)-3-carboxylic acid {(R)-1-(4-chloro-benzyl)-2-[1,3-dioxo-8a-pyridin-2-ylmethyl-2-(2,2,2-trifluoro-ethyl)-hexahydro-imidazo[5-a]pyrazin-7-yl]-2-oxo-ethyl}-amide

To a solution of N-Boc-L-Tic-OH (1g, 3.6 mmol) in CH2Cl2 (20 mL) was added triethyl amine ( 0.5 mL), EDC (726 mg, 3.8 mmol) and N-hydroxysuccinimide (437 mg, 3.8 mmol), respectively. The resulting solution was stirred at rt for 4 h, diluted with water (20 mL) and extracted with CH2Cl2 (3×20 mL). The combined extracts were washed with citric acid, saturated NaHCO3 and brine solutions, dried over MgSO4 and evaporated to give 1.18g 3,4-Dihydro-1H-isoquinoline-23-(S)-dicarboxylic acid 2-tert-butyl ester 3-(2,5-dioxo-pyrrolidin-1-yl) ester. To a solution of 3,4-Dihydro-1H-isoquinoline-23-(S)-dicarboxylic acid 2-tert-butyl ester 3-(2,5-dioxo-pyrrolidin-1-yl) ester (187 mg, 0.5 mmol) in CH2CI2 (10 mL) was added triethyl amine (0.13 mL) and D-para-chloro-phenylalanine (100 mg, 0.5 mmol). The resulting solution was stirred at rt overnight, diluted with water (20 mL) and extracted with CH2Cl2 (3×10 mL). The combined extracts were washed with citric acid and brine solutions, dried over MgSO4 and evaporated to give 134 mg 3-(S)-[(R)-1-Carboxy-2-(4-chloro-phenyl)-ethylcarbamoyl]-3,4-dihydro-1H-isoquinoline-2-carboxylic acid tert-butyl ester. To a solution of 3-(S)-[(R)-1-Carboxy-2-(4-chloro-phenyl)-ethylcarbamoyl]-3,4-dihydro-1 H-isoquinoline-2-carboxylic acid tert-butyl ester ( 23 mg, 0.05 mmol) in CH2Cl2 (5 mL) was added TEA (30 uL) and EDC (12 mg, 0.06 mmol). After stirring at 0 C for 15 min, 8a-Pyridin-2-ylmethyl-2-(2,2,2-trifluoro-ethyl)-tetrahydro-imidazo[1,5-a]pyrazine-1,3-dione (22 mg, 0.05 mmol) was added and the resulting solution was stirred for 5h, diluted with water (10 mL) and extracted with CH2Cl2 (3×10 mL). The combined extracts were washed with saturated NaHCO3 and brine solutions, dried over MgSO4 and evaporated. Crude oil was purified (SiO2 gel/ 4:1 EtOAc/hexanes) to deliver 10 mg (S)-3-{ (R)-1-(4-Chloro-benzyl)-2-[1,3-dioxo-8a-pyridin-2-ylmethyl-2-(2,2,2-trifluoro-ethyl)-hexahydro-imidazo[1,5-a]pyrazin-7-yl]-2-oxo-ethylcarbamoyl}-3,4-dihydro-1 H-isoquinoline-2-carboxylic acid tert-butyl ester. This product (8 mg) was dissolved into in EtOH (2 mL), treated with 0.25 mL conc HCL and stirred at 0 C for 0.5h. The solution was evaporated to dryness and the resulting residue was triturated with ether to give 6 mg of the HCl salt. . MS/+: 669.1; MS/−: 667.2

### Example 2

### 1,2,3,4-Tetrahydro-isoquinoline-(R)-3-carboxylic acid {(R)-1-(4-chloro-benzyl)-2-1,3-dioxo-8a-pyridin-2-ylmethyl-2-(2,2,2-trifluoro-ethyl)-hexahydro-imidazo[1,5-a]pyrazin-7-yl]-2-oxo-ethyl}-amide

To a solution of N-Boc-D-Tic-OH (277 mg, 1.0 mmol) in CH2Cl2 (10 mL) was added triethyl amine ( 0.26 mL), EDC (219 mg, 1.2 mmol) and N-hydroxysuccinimide (126 mg, 1.1 mmol), respectively. The resulting solution was stirred at rt overnight, diluted with water (10 mL) and extracted with CH2Cl2 (3×10 mL). The combined extracts were washed with citric acid, saturated NaHCO3 and brine solutions, dried over MgSO4 and evaporated to give 311 mg 3,4-Dihydro-1H-isoquinoline-2,3-(R)-dicarboxylic acid 2-tert-butyl ester 3-(2,5-dioxo-pyrrolidin-1-yl) ester. To a solution of 3,4-Dihydro-1H-isoquinoline-2,3-(R)-dicarboxylic acid 2-tert-butyl ester 3-(2,5-dioxo-pyrrolidin-1-yl) ester (187 mg, 0.5 mmol) in CH2Cl2 (10 mL) was added triethyl amine (0.13 mL) and D-para-chloro-phenylalanine (100 mg, 0.5 mmol). The resulting solution was stirred at rt overnight, diluted with water (20 mL) and extracted with CH2Cl2 (3×10 mL). The combined extracts were washed with citric acid and brine solutions, dried over MgSO4 and evaporated to give 229 mg 3-(R)-[(R)-1 -Carboxy-2-(4-chloro-phenyl)-ethylcarbamoyl]-3,4-dihydro-1 H-isoquinoline-2-carboxylic acid tert-butyl ester. 3-(R)-[(R)-1 -Carboxy-2-(4-chloro-phenyl)-ethylcarbamoyl]-3,4-dihydro-1 H-isoquinoline-2-carboxylic acid tert-butyl ester ( 23 mg, 0.05 mmol) in CH2Cl2 (5 mL) was added TEA (30 uL) and EDC (12 mg, 0.06 mmol). After stirring at 0 C for 15 min, 8a-Pyridin-2-ylmethyl-2-(2,2,2-trifluoro-ethyl)-tetrahydro-imidazo[1,5-a]pyrazine-1,3-dione (22 mg, 0.05 mmol) was added and the resulting solution was stirred for 5 h, diluted with water (10 mL) and extracted with CH2Cl2 (3×10 mL). The combined extracts were washed with saturated NaHCO3 and brine solutions, dried over MgSO4 and evaporated. Crude oil was purified (SiO2 gel/4:1 EtOAc/hexanes) to deliver 11 mg 3-(R)-{ (R)-1-(4-Chloro-benzyl)-2-[1,3-dioxo-8a-pyridin-2-ylmethyl-2-(2,2,2-trifluoro-ethyl)-hexahydro-imidazo[1,5-a]pyrazin-7-yl]-2-oxo-ethylcarbamoyl}-3,4-dihydro-1 H-isoquinoline-2-carboxylic acid tert-butyl ester. This product (10 mg) was dissolved into in EtOH (2 mL), treated with 0.25 mL conc HCL and stirred at 0 C for 0.5 h. The solution was evaporated to dryness and the resulting residue was triturated with ether to give 8 mg of the HCl salt. MS/+: 669.2; MS/−: 667.2

Example3

### 1,2,3,4-Tetrahydro-isoquinoline-(S)-3-carboxylic acid r2-(3a-benzyl-2-methyl-3-oxo-2,3,3a,4,6,7-hexahydro-pyrazolo[4,3-cl pyridin-5-yl)-1 -(4-chloro-benzyl)-2-oxo-ethyl}-amide

To a solution of (S)-3-[(R)-1-Carboxy-2-(4-chloro-phenyl)-ethylcarbamoyl]-3,4-dihydro-1H-isoquinoline-2-carboxylic acid tert-butyl ester ( 23 mg, 0.05 mmol) in EtOAc (5 mL) was added TEA (30 uL) and PPAA (35 uL, 0.055 mmol, 50% solution in EtOAc). After stirring at 0 C for 5 min, a cooled solution of 3a-Benzyl-2-methyl-2,3a,4,5,6,7-hexahydro-pyrazolo[4,3-c]pyridin-3-one (13 mg, 0.055 mmol) in EtOAc (1 mL) was added and the resulting solution was stirred for 4 h, diluted with water (10 mL) and extracted with EtOAc (3×10 mL). The combined extracts were washed with saturated NaHCO3 and brine solutions, dried over MgSO4 and evaporated. Crude oil was purified (SiO2 gel/ 3:1 EtOAc/hexanes) to deliver 13 mg of Boc-protected adduct. This material was dissolved into EtOH (2 mL), cooled in an ice bath and treated with conc. HCL (0.25 mL) for 30 min. Evaporate and tritrate with ether to give 10 mg of the HCl salt. MS/+:584.2; MS/−:582.1

### Example4

### 1,2,3,4-Tetrahydro-isoquinoline-(R)-3-carboxylic acid [2-(3a-benzyl-2-methyl-3-oxo-2,3,3a,4,6,7-hexahydro-pyrazolo[4,3-c]pyridin-5-yl)-1 -(4-chloro-benzyl)-2-oxo-ethyl]-amide

To a solution of (S)-3-[(R)-1-Carboxy-2-(4-chloro-phenyl)-ethylcarbamoyl]-3,4-dihydro-1H-isoquinoline-2-carboxylic acid tert-butyl ester ( 46 mg, 0.05 mmol) in EtOAc (5 mL) was added TEA (70 uL) and PPAA (70uL, 0.11 mmol, 50% solution in EtOAc). After stirring at 0 C for 5 min, a cooled solution of 3a-Benzyl-2-methyl-2,3a,4,5,6,7-hexahydro-pyrazolo[4,3-c]pyridin-3-one (26 mg, 0.11 mmol) in EtOAc (1 mL) was added and the resulting solution was stirred for 4 h, diluted with water (10 mL) and extracted with EtOAc (3×10 mL). The combined extracts were washed with saturated NaHCO3 and brine solutions, dried over MgSO4 and evaporated. Crude oil was purified (SiO2 gel/3:1 EtOAc/hexanes) to deliver 28 mg of Boc-protected adduct. This material was dissolved into EtOH (2 mL), cooled in an ice bath and treated with conc. HCL (0.25 mL) for 30 min. Evaporate and tritrate with ether to give 21 mg of the HCl salt. MS/+: 584.2; MS/−: 582.1

1. A compound of the formula 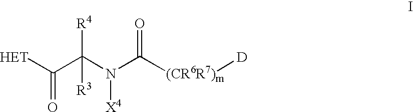
or a stereoisomeric mixture thereof, diastereomerically enriched, diastereomerically pure, enantiomerically enriched or enantiomerically pure isomer thereof, or a prodrug of such compound, mixture or isomer thereof, or a pharmaceutically acceptable salt of the compound, mixture, isomer or prodrug,
wherein:
m is 0, 1 or 2;
HET is a heterocyclic moiety selected from the group consisting of 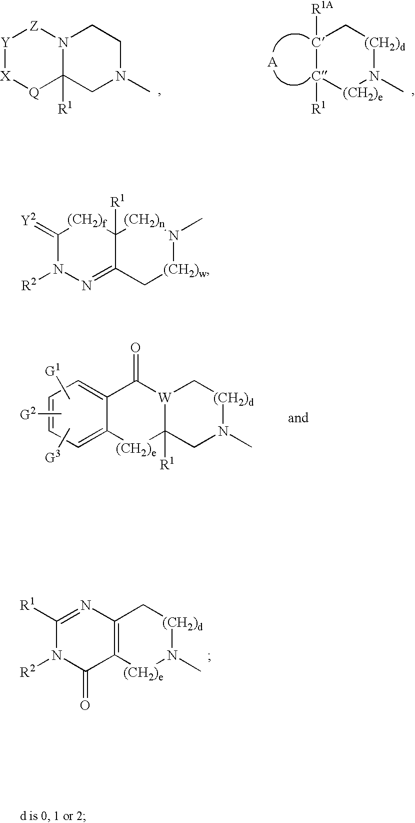
e is 1 or 2;
f is 0 or 1;
n and w are 0, 1 or 2, provided that n and w cannot both be 0 at the same time;
y² is oxygen or sulfur;
A is a radical, where the left hand side of the radical as shown below is connected to C″ and the right hand side of the radical as shown below is connected to C′, selected from the group consisting of —NR²—C(O)—NR²—, —NR²—S(O)₂—NR²—, —O—C(O)—NR²—, —NR²—C(O)—O—, —C(O)—NR²—C(O)—, —C(O)—NR²—C(R⁹R¹⁰)—, —C(R⁹R¹⁰)—NR²—C(O)—, —C(R⁹R¹⁰)—C(R⁹R¹⁰)—C(R⁹R¹⁰)—, —S(O)₂—C(R⁹R¹⁰)—C(R⁹R¹⁰)—, —C(R⁹R¹⁰)—O—(O)—, —C C(R⁹R¹⁰)—O—C(R⁹R¹⁰)—, —NR²—C(O)—C(R⁹R¹⁰)—, —O—C(O)—C(R⁹R¹⁰)—, —C(R⁹R¹⁰ )—C (O)—NR²—, —C(O)—NR²—C(O)—, —C(R⁹R¹⁰)—C(O)—O—, —C(O)—NR²—C(R⁹R¹⁰)—C(R⁹R¹⁰)—, —C(O)—O—C(R⁹R¹⁰)—, —C(R⁹R¹⁰)—C(R⁹R¹⁰)—C(R⁹R¹⁰)—C(R⁹R¹⁰)—, —S(O)₂—NR²—C(R⁹R¹⁰)—C(R⁹R¹⁰)—, —C(R⁹R¹⁰)—C(R⁹R¹⁰)—NR²C(O)—, —C( R⁹R¹⁰)—C(R⁹R¹⁰ )—O—C(O)—, —NR²—C(O)—C(R⁹R¹⁰)—, C(R⁹R¹⁰)—, —NR²—S(O)₂—C(R⁹R¹⁰)—C(R⁹R¹⁰)—, —O—C(O)—C(R⁹R¹⁰)—C(R⁹R¹⁰)——C(R⁹R¹⁰)—C(R⁹R¹⁰)—C(O)—NR²—, —C(R⁹R¹⁰)—C(R⁹R¹⁰)—C(O)—, —C(R⁹R¹⁰)—NR²—C(O)—O—, —C(R⁹R¹⁰)—O—C(O)—NR², —C(R⁹R¹⁰)—NR²—C(O)—NR²—, —NR²—C(O)—O—C(R⁹R¹⁰)—, —NR²—C(O)—NR²—C(R⁹R¹⁰)—, —NR²—S(O)₂—NR²—C(R⁹R¹⁰)—, —O—C(O)—NR²—C(R⁹R¹⁰)—, —C(O)—N═C(R¹¹)—NR²—, —C(O)—NR²—C(R¹¹)═N—, —C(R⁹R¹⁰)—NR¹²—C(R⁹R¹⁰)—, —NR¹²—C(R⁹R¹⁰)—, —NR 12—C(R⁹R¹⁰)—C(R⁹R¹⁰)—, —C(O)—O—C(R⁹R¹⁰ )—C(R⁹R¹⁰ )—, —NR²—C(R¹¹)═N—C(O)—, —C(R⁹R¹⁰)—C(R⁹R¹⁰)—N(R¹²)—, —C(R⁹R¹⁰)—NR¹²—, —N═C(R¹¹)—NR —C(O)—, —C(R⁹R¹⁰)—C(R⁹R¹⁰)—NR²—S(O)₂—, —C(R⁹R¹⁰)—C(R⁹R¹⁰)—S(O)₂—NR²—, —C(R⁹R¹⁰)—C(R⁹R¹⁰)—C(O)—O—, —C(R⁹R¹⁰)—S(O)₂—C(R⁹R¹⁰)—, —C(R⁹R¹⁰)—C(R⁹R¹⁰)—S(O)₂—, —O—C(R⁹R¹⁰)—C(R⁹R¹⁰)—, —C(R⁹R¹⁰)—C(R⁹R¹⁰)—O—, —C(R⁹R¹⁰)—C(O)—C(R⁹R¹⁰)—, —C(O)—C(R⁹R¹⁰)—C(R⁹R¹⁰)—and —C(R⁹R¹⁰)—NR²—S(O)₂—NR²—;
Q is a covalent bond or CH₂;
W is CH or N;
X is CR⁹R¹⁰, C═CH₂ or C═O;
Y is CR⁹R¹⁰, O or NR²;
Z is C═O, C═S or S(O)₂;
G¹ is hydrogen, halo, hydroxy, nitro, amino, cyano, phenyl, carboxyl, —CONH₂, —(C₁-C₄)alkyl optionally independently substituted with one or more phenyl, one or more halogens or one or more hydroxy groups, —(C₁-C₄)alkoxy optionally independently substituted with one or more phenyl, one or more halogens or one or more hydroxy groups, —(C₁-C₄)alkylthio, phenoxy, —COO(C₁-C₄)alkyl, N,N—di—(C₁-C₄) alkylamino, —(C₂-C₆)alkenyl optionally independently substituted with one or more phenyl, one or more halogens or one or more hydroxy groups, —(C₂-C₆)alkynyl optionally independently substituted with one or more phenyl, one or more halogens or one or more hydroxy groups, —(C₃-C₆)cycloalkyl optionally independently substituted with one or more (C₁-C₄)alkyl groups, one or more halogens or one or more hydroxy groups, —(C₁-C₄)alkylamino carbonyl or di—(C₁-C₄)alkylamino carbonyl;
G² and G³ are each independently selected from the group consisting of hydrogen, halo, hydroxy, —(C₁-C₄)alkyl optionally independently substituted with one to three halogens and —(C₁-C₄)alkoxy optionally independently substituted with one to three halogens;
R¹ is hydrogen, —CN, —(CH₂)_{q}N(X⁶)C(O)X⁶, —(CH₂)_{q}N(X⁶)C(O)(CH₂)ₜ—A¹, —(CH₂)_{q}N (X⁶)S(O)₂(CH₂)ₜ—A¹, —(CH₂)_{q}N(X⁶)S(O)₂X⁶, —(CH₂)_{q}N(X⁶)C(O)N (X⁶)CH₂)ₜ—A¹, —(CH₂)_{q}N (X⁶)C(O)N(X⁶)(X⁶), —(CH₂)_{q}N(O)N (X⁶)(X⁶), —(CH₂)_{q}C(O)N (X⁶)(CH₂)ₜ—A¹, —(CH₂)_{q}C(O)OX⁶, —(CH₂)_{q}C(O)O(CH₂) ₜ—A¹, —(CH₂)_{q}OX⁶, —(CH₂)_{q}OC(O)X⁶, —(CH₂)_{q}OC(O)(CH₂)ₜ—A¹,—(CH₂) _{q}OC(O)N (X⁶)(CH₂)ₜ—A¹, —(CH₂)_{q}OC(O)N(X⁶)(X⁶), —(CH₂)_{q}C(O )X⁶, —(CH₂) _{q}C(O)(CH₂)ₜ—A¹, —(CH₂)_{q}N(X⁶)C(O)OX⁶, —(CH₂)_{q}N(X⁶)S(O)₂N(X⁶)(X⁶), —(CH₂) _{q}S(O)ₘX⁶, —(CH₂)_{q}S(O)ₘ(CH₂)ₜA¹, —(C₁-C₁₀)alkyl, —(CH₂)ₜ—A¹, —(CH₂) _{q}—(C₃-C₇)cycloalkyl, —(CH₂)_{q}—Y¹—(C¹-C₆)alkyl, —(CH₂)_{q}—Y¹—(CH₂)ₜ—A¹ or —(CH₂)_{q}—Y¹—(CH₂)ₜ—(C₃-C₇)cycloalkyl;
where the alkyl and cycloalkyl groups in the definition of R¹ are optionally substituted with (C₁-C₄)alkyl, hydroxy, (C₁-C₄)alkoxy, carboxyl, —CONH₂, —S(O)ₘ(C₁-C₆)alkyl, —CO₂(C₁-C₄) alkyl ester, 1 H-tetrazol-5-yl or 1, 2 or 3 fluoro groups;
Y¹ is O, S(O)ₘ, —C(O)NX⁶—, —CH═CH—, —C≡C—, —N (X⁶)C(O)—, —C(O)NX⁶—, —C(O)O—, —OC(O)N(X⁶)—or —OC(O)—;
q is 0,1,2,3 or4;
tis O, 1,2or 3;
said (CH₂)_{q} group and (CH₂)ₜ group in the definition of R¹ are optionally independently substituted with hydroxy, (C₁-C₄)alkoxy, carboxyl, —CONH₂, —S(O)ₘ(C₁-C₆)alkyl, —CO₂(C₁-C₄) alkyl ester, 1 H-tetrazol-5-yl, 1, 2 or 3 fluoro groups or 1 or 2 (C₁-C₄)alkyl groups;
R^{1A} is selected from the group consisting of hydrogen, F, Cl, Br, I, (C₁-C₆) alkyl, phenyl(C₁-C₃)alkyl, pyridyl(C₁-C₃)alkyl, thiazolyl(C₁-C₃)alkyl and thienyl(C₁C₃) alkyl, provided that R^{1A} is not F, Cl, Br or I when a heteroatom is vicinal to C″;
R², for each occurrence, is independently hydrogen, (C₁-C₈)alkyl, —(C₀-C₃) alkyl—(C₃-C₈)cycloalkyl, —(C₁-C₄)alkyl-A¹ or A¹;
where the alkyl groups and the cycloalkyl groups in the definition of R² are optionally substituted with hydroxy, —C(O)OX⁶, —C(O)N (X⁶)(X⁶), —N(X⁶)(X⁶), —S(O)ₘ(C₁-C₆)alkyl, —C(O)A¹, —C(O)(X⁶), CF₃, CN or 1, 2 or 3 independently selected halogens;
R³ and R⁴ are each independently selected from the group consisting of hydrogen, (C₁-C₈)alkyl, —CH(R⁸)-aryl, —CH(R⁸)-heteroaryl, —(C₀-C₃)alkyl(C₃-C₈) cycloalkyl, wherein the aryl or heteroaryl groups are optionally substituted by one or two R^{b} groups;
R^{b}, for each occurrence independently, is R^{c}, halo, —OR^{C}, —NHSO₂R^{C}, —N(R^{c})₂, —CN, —NO₂, —SO₂N(R^{c})₂, —SO₂R^{c}, —CF₃, —OCF₃; —OCF₂H or two R^{b} groups attached to adjacent carbon atoms taken together to form methylenedioxy;
R^{c}, for each occurrence independently, is hydrogen, —(C₁-C₈)alkyl, —(C₀-C₃) alkylaryl, —(C₀-C₃)alkylheteroaryl, (C₃-C₆)cycloalkyl; or 2 R^{b} taken together with the nitrogen atom to which they are attached to form a 5- or 6-membered ring optionally containing an additional heteroatom selected from O, S or NR³;
R⁶ and R⁷ are each independently selected from hydrogen, (C₁-C₆)alkyl, —(C₀-C₃) alkylaryl, —(C₀-C₃)alkylheteroaryl, —(C₀-C₃)alkyl(C₃-C₈)cycloalkyl;
or R⁶ and R⁷ together with the nitrogen atom to which they are attached form a 5- or 6-membered ring optionally containing an additional heteroatom selected from O, S, NR³;
D is —(C₀-C₆)alkyl-amino-C(═N R⁷)—NR¹⁵R¹⁶, —(C₀-C₆)alkylaminopyridyl, —(C₀-C₆) alkylaminoimidazolyl, —(C₀-C₆)alkylaminothiazolyl, —(C₀-C₆)alkylaminopyrimidinyl, (C₀-C₆)alkylaminopiperazinyl-R¹⁵, —(C0-C₆)alkylmorpholinyl, wherein R¹⁵ and R¹⁶ are independently hydrogen, —(C₁-C₆)alkyl, —(C₀-C₃)alkylaryl, (C₀-C₃)alkylheteroaryl, (C₀-C₃)alkyl(C₃-C₈) cycloalkyl, wherein the alkyl and aryl groups are optionally substituted with one or two R^{b} groups; or D is a group of the formula 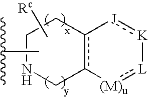
wherein the dashed lines represent optional double bonds;
u is 0 or 1;
x and y are each independently 0, 1 or 2;
J, K, L and M are each independently selected from C(R^{b})ᵣ, N, S or O wherein R^{b} and R^{c} are as defined above and r is 1 or 2;
X⁴ is hydrogen or (C₁-C₆)alkyl or X⁴ is taken together with R⁴ and the nitrogen atom to which X⁴ is attached and the carbon atom to which R⁴ is attached and form a five to seven membered ring;
R⁸ is hydrogen, —(C₁-C₈)alkyl, —(C₀-C₃)alkylaryl, —(C₀-C₃)alkylheteroaryl, —(C₃-C₆) cycloalkyl; or 2 R^{b} taken together with the nitrogen atom to which they are attached to form a 5- or 6-membered ring optionally containing an additional heteroaryl selected from O, S or NR³;
R⁹ and R¹⁰, for each occurrence, are each independently selected from the group consisting of hydrogen, fluoro, hydroxy and (C₁-C₅)alkyl optionally independently substituted with 1-5 halogens;
R¹¹ is selected from the group consisting of (C₁-C₅)alkyl and phenyl optionally substituted with 1-3 substituents each independently selected from the group consisting of (C₁-C₅)alkyl, halo and (C₁-C₅)alkoxy;
R¹² is selected from the group consisting of (C₁-C₅)alkylsulfonyl, (C₁-C₅) alkanoyl and (C₁-C₅)alkyl where the alkyl portion is optionally independently substituted by 1-5 halogens;
A¹ for each occurrence is independently selected from the group consisting of (C₅-C₇)cycloalkenyl, phenyl, a partially saturated, fully saturated or fully unsaturated 4- to 8-membered ring optionally having 1 to 4 heteroatoms independently selected from the group consisting of oxygen, sulfur and nitrogen and a bicyclic ring system consisting of a partially saturated, fully unsaturated or fully saturated 5- or 6-membered ring, optionally having 1 to 4 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
A¹ for each occurrence is independently optionally substituted, on one or optionally both rings if A¹ is a bicyclic ring system, with up to three substituents, each substituent independently selected from the group consisting of F, Cl, Br, I, —OCF₃, —OCF₂H, —CF₃, —CH₃, —OCH₃, —OX⁶, —C(O)N(X⁶)(X⁶), —C(O)OX⁶, oxo, (C₁-C₆)alkyl, nitro, cyano, benzyl, —S(O)ₘ(C₁-C₆)alkyl, 1 H-tetrazol-5-yl, phenyl, phenoxy, phenylalkyloxy, halophenyl, methylenedioxy, —N(X⁶)(X⁶), —N(X⁶)C(O)(X⁶), —S(O)₂N(X⁶)(X⁶), —N(X⁶)S(O)₂-phenyl, —N(X⁶)S(O)₂X⁶, —CONX¹¹ X ¹², —S(O)₂NX¹¹X^{12,} —NX⁶S(O)₂X¹², —NX⁶CONX¹¹X¹², —NX⁶S(O)₂NX¹¹X¹², —NX⁶C(O)X¹², imidazolyl thiazolyl and tetrazolyl, provided that if A¹ is optionally substituted with methylenedioxy then it can only be substituted with one methylenedioxy;
where X¹¹, for each occurrence, is independently hydrogen or optionally substituted (C₁-C₆)alkyl;
the optionally substituted (C₁-C₆)alkyl defined for X¹¹ is optionally independently substituted with phenyl, phenoxy, (C₁-C₆) alkoxycarbonyl, -S(O)ₘ(C₁-C₆)alkyl, 1 to 5 halogens, 1 to 3 hydroxy groups, 1 to 3 (C₁-C₁₀)alkanoyloxy groups or 1 to 3 (C₃-C₆)alkoxy groups;
X¹², for each occurrence, is independently hydrogen, (C₁-C₆)alkyl, phenyl, thiazolyl, imidazolyl, furyl or thienyl, provided that when X¹² is not hydrogen, the X¹² group is optionally substituted with one to three substituents independently selected from the group consisting of Cl, F, CH₃, OCH₃, OCF₃ and CF₃;
or X¹¹ and X¹² are taken together to form —(CH₂)_{g}—L¹—(CH₂)_{g}—;
L¹ is C(X²)(X²), O, S(O)ₘ or N(X²);
g for each occurrence is independently 1, 2 or 3;
X² for each occurrence is independently hydrogen, optionally substituted (C₁-C₆) alkyl or optionally substituted (C₃-C₇)cycloalkyl, where the optionally substituted (C₁-C₆)alkyl and optionally substituted (C₃-C₇)cycloalkyl in the definition of X² are optionally independently substituted with —S(O)ₘ(C₁-C₆)alkyl, —C(O)OX³, 1 to 5 halogens or 1-3 OX³ groups;
X³ for each occurrence is independently hydrogen or (C₁-C₆)alkyl;
X⁶ for each occurrence is independently hydrogen, optionally substituted (C₁-C₆) alkyl, (C₂-C₆)halogenated alkyl, optionally substituted (C₃-C₇)cycloalkyl, (C₃-C₇)- halogenated cycloalkyl, where optionally substituted (C₁-C₆)alkyl and optionally substituted (C₃-C₇)cycloalkyl in the definition of X⁶ is optionally independently mono- or di-substituted with (C₁-C₄)alkyl, hydroxy, (C₁-C₄)alkoxy, carboxyl, CONH₂, —S(O)ₘ(C₁-C₆)alkyl, carboxylate (C₁-C₄)alkyl ester or 1 H-tetrazol-5-yl; or when there are two X⁶ groups on one atom and both X⁶ are independently (C₁-C₆) alkyl, the two (C₁-C₆)alkyl groups may be optionally joined and, together with the atom to which the two X⁶ groups are attached, form a 4- to 9-membered ring optionally having oxygen, sulfur or NX⁷ as a ring member;
X⁷ is, for each occurrence independently, hydrogen or (C₁-C₆)alkyl optionally substituted with hydroxy;
m for each occurrence is independently 0, 1 or 2;
with the proviso that: X⁶ and X¹² cannot be hydrogen when attached to C(O) or S(O)₂ in the form C(O)X⁶, C(O)X¹², S(O)₂X⁶ or S(O)₂X¹².

2. A compound according to claim 1, wherein D is

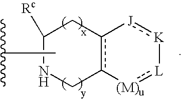

3. A compound according to claim 2, wherein x is 1, y is 1 and u is 1.

4. A compound according to claim 3, wherein J, k, L and M are each NR^{b} or C(R^{b})ᵣ where r=1 or 2, R ⁴ is —CH₂-aryl in which aryl is optionally substituted by R^{b}

5. A compound according to claim 4, wherein HET is

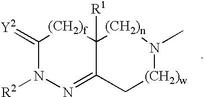

6. A compound according to claim 5, wherein y² is oxygen, f is 0, n is 1 or 2; and w is 0 or 1.

7. A compound according to claim 6, wherein R² is (C₁-C₆)alkyl optionally substituted by halo, R³ is hydrogen, n is 1, w is 1, and R¹ is aryl(C₁-C₆)alkyl, (C₁-C₆) alkyl or heteroaryl(C₁-C₆)alkyl wherein aryl and heteroaryl are optionally substituted with one or two groups from the following list: halo, —OR^{c}, —NHSO₂R^{c}, —N(R^{c})₂ , —CN, —NO₂, —SO₂N(R^{c})₂, —SO₂R^{c}, —CF₃, —OCF₃; —OCF₂H.

8. A compound according to claim 7, wherein J, K, L and M are each N or CR^{b} and the dashed lines represent double bonds, R¹ is benzyl optionally substituted by halo, —R^{c}, —OR^{c}, —CF₃, —OCF₃, —OCF₂H, R^{c}, hydrogen, —(C₁-C₆)alkyl, —(C₀-C₃)alkylaryl, —(C₀-C₃)alkylheteroaryl or —(C₃-C₆)cycloalkyl.

9. A compound according to claim 1, wherein said compound is selected from the group consisting of:
1,2,3,4-Tetrahydro-isoquinoline-(S)3-carboxylic acid [2-((R)3a-benzyl-2-methyl-3-oxo-2,3,3a,4,6,7-hexahydro-pyrazolo[4,3-c]pyridin-5-yl) -(R)1 -(4-chloro-benzyl)-2-oxo-ethyl]-amide;
1,2,3,4-Tetrahydro-isoquinoline-(R)3-carboxylic acid [2-((R3a-benzyl-2-methyl-3-oxo-2,3,3a,4,6,7-hexahydro-pyrazolo[4,3-c]pyridin-5-yl) -(R)1 -(4-chloro-benzyl)-2-oxo-ethyl]-amide;
1,2,3,4-Tetrahydro-isoquinoline-(R)3-carboxylic acid [2-[3a-benzyl-3-oxo-2-(2,2,2-trifluoro-ethyl)-2,3,3a,4,6,7-hexahydro-pyrazolo[4,3-c]pyridin-5-yl]-(R)1 -(4-chloro-benzyl)-2-oxo-ethyl]-amide;
1,2,3,4-Tetrahydro-isoquinoline-(R)3-carboxylic acid {(R)1-(4-chloro-benzyl)-2-[2-ethyl-(S)3a-(4-fluoro-benzyl)-3-oxo-2,3,3a,4,6,7-hexahydro-pyrazolo[4,3-c]pyridin-5-yl]-2-oxo-ethyl}-amide;
1,2,3,4-Tetrahydro-isoquinoline-(S)3-carboxylic acid {(R)1-(4-chloro-benzyl)-2-[2-ethyl-(S)3a-(4-fluoro-benzyl)-3-oxo-2,3,3a,4,6,7-hexahydro-pyrazolo[4,3-c]pyridin-5-yl]-2-oxo-ethyl}-amide;
1,2,3,4-Tetrahydro-isoquinoline-(S)3-carboxylic acid {(R)1-(4-chloro-benzyl)-2-[(S)3a-(4-chloro-benzyl)-2-ethyl-3-oxo-2,3,3a,4,6,7-hexahydro-pyrazolo[4,3-c]pyridin-5-yl]-2-oxo-ethyl}-amide;
1,2,3,4-Tetrahydro-isoquinoline-(R)3-carboxylic acid {(R)1-(4-chloro-benzyl)-2-[(S)3a-(4-chloro-benzyl)-2-ethyl-3-oxo-2,3,3a,4,6,7-hexahydro-pyrazolo[4,3-c]pyridin-5-yl]-2-oxo-ethyl}-amide;
1,2,3,4-Tetrahydro-isoquinoline-(R)3-carboxylic acid [2-((S)3a-benzyl-2-methyl-3-oxo-2,3,3a,4,6,7-hexahydro-pyrazolo[4,3-c]pyridin-5-yl)-(R)1-(4-chloro-benzyl)-2-oxo-ethyl]-amide;
1,2,3,4-Tetrahydro-isoquinoline-(R)3-carboxylic acid {(R)1-(4-chloro-benzyl)-2-[(R)3a-(3-fluoro-benzyl)-3-oxo-2,3,3a,4,6,7-hexahydro-pyrazolo[4,3-c]pyridin-5-yl]-2-oxo-ethyl}-amide;
1,2,3,4-Tetrahydro-isoquinoline-(S)3-carboxylic acid [2-[3a-benzyl-3-oxo-2-(2,2,2-trifluoro-ethyl)-2,3,3a,4,6,7-hexahydro-pyrazolo[4,3-c]pyridin-5-yl]-(R)1-(4-chloro-benzyl)-2-oxo-ethyl]-amide; and
1,2,3,4-Tetrahydro-isoquinoline-(R)3-carboxylic acid [(R)1-(4-chloro-benzyl)-2-oxo-2-(3-oxo-3a-pyridin-2-yl methyl-2 ,3,3a ,4,6,7-hexahydro-pyrazolo[4,3-c]pyridin-5-yl)-ethyl]-amide.

10. A compound according to claim 7, wherein J, K, L and M are each NR^{b} or C(R^{b})₂ and the dashed lines represent single bonds, wherein R^{b} is hydrogen, halo, R^{c}, —OR^{C}, —CF₃, —OCF₃, —OCF₂H, R^{c} is hydrogen, (C₁-C₈)alkyl, (C₀-C₃)alkylaryl, (C₀-C₃)alkylheteroaryl or —(C₃-C₆)cycloalkyl.

11. A compound according to claim 4, wherein HET is

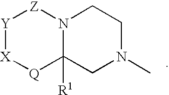

12. A compound according to claim 11, wherein Q is a covalent bond; X and Z are each C═O; and Y is NR².

13. A compound according to claim 12, wherein R² is (C₁-C₆)alkyl optionally substituted by halo, and R¹ is aryl(C₁-C₆)alkyl, (C₁-C₆)alkyl or heteroaryl (C₁-C₆)alkyl wherein aryl and heteroaryl are optionally substituted with one or two groups from the following list: halo, OR^{c}, —NHSO₂R^{c}, N(R^{c})₂, CN, NO₂, SO₂N(R^{c})₂, —SO₂R^{c}, —CF₃, —OCF₃, —OCF₂H.

14. A compound according to claim 13, wherein J, K, L and M are each N or CR^{b} and the dashed lines represent double bonds, R¹ is benzyl optionally substituted by halo, —R^{c}, —OR^{c}, —OCF₃, —OCF₂H, and R^{C} is hydrogen, —(C₁-C₈)alkyl, —(C₀-C₃)akylaryl, —(C₀-C₃)alkylheteroaryl or —(C₃-C₆)cycloalkyl.

15. A compound according to claim 1, wherein said compound is selected from the group consisting of:
1,2,3,4-Tetrahydro-isoquinoline-(S)3-carboxylic acid {(R)1-(4-chloro-benzyl)-2-[1,3-dioxo-(S)8a-pyridin-2-ylmethyl-2-(2,2,2-trifluoro-ethyl)-hexahydro-imidazo[1,5-a]pyrazin-7-yl]-2-oxo-ethyl}-amide;
1,2, 3,4-Tetrahydro-isoquinoline-(R)3-carboxylic acid {(R) 1 -(4-chloro-benzyl)-2-[(R)8a-(4-fluoro-benzyl)-2-methyl-1,3-dioxo-hexahydro-imidazo[1,5-a]pyrazin-7-yl]-2-oxo-ethyl}-amide;
1,2,3,4-Tetrahydro-isoquinoline-(S)3-carboxylic acid {(R)1-(4-chloro-benzyl)-2-[1,3-dioxo-(S)8a-pyridin-3-ylmethyl-2-(2,2,2-trifluoro-ethyl)-hexahydro-imidazo[1,5-a]pyrazin-7-yl]-2-oxo-ethyl}-amide;
1,2,3,4-Tetrahydro-isoquinoline-(S)3-carboxylic acid {(R)1-(4-chloro-benzyl)-2-[8a-(4-fluoro-benzyl)-3-oxo-tetrahydro-oxazolo[3,4-a]pyrazin-7-yl]-2-oxo-ethyl}-amide;
1,2,3,4-Tetrahydro-isoquinoline-(S)3-carboxylic acid {(R)1-(4-chloro-benzyl)-2-[8a-(4-fluoro-benzyl)-2-methyl-1,3-dioxo-hexahydro-imidazo[1,5-a]pyrazin-7-yl]-2-oxo-ethyl}-amide; and
1,2,3,4-Tetrahydro-isoquinoline-(S)3-carboxylic acid {(R)1-(4-chloro-benzyl)-2-[8a-(4-fluoro-benzyl)-2-methyl-1,3-dioxo-hexahydro-imidazo[1,5-a]pyrazin-7-yl]-2-oxo-ethyl}-amide.

16. A compound according to claim 13, wherein J, K, L and M are each NR^{b} or C(R^{b})₂ and the dashed lines represent single bonds, R^{b} is hydrogen, halo, R^{c}, OR^{c}, —CF₃, —OCF₃, —OCF₂H, R^{C} is hydrogen, —C₁-C₈)akyl, —(C₀-C₃)alkylaryl, —(C₀-C₃) alkylheteroaryl or —(C₃-C₆)cycloalkyl.

17. A method for the treatment or prevention of disorders, diseases or conditions responsive to the activation of melanocortin receptor which comprises administering to a mammal in need of such treatment or prevention an effective amount of a compound of claim 1.

18. A method for the treatment or prevention of obesity which comprises administering to a mammal in need of such treatment or prevention an effective amount of a compound of claim 1.

19. A method for the treatment or prevention of diabetes mellitus which comprises administering to a mammal in need of such treatment or prevention an effective amount of claim 1.

20. A method for the treatment or prevention of male or female sexual dysfunction which comprises administering to a mammal in need of such treatment or prevention an effective amount of a compound of claim 1.

21. A method for the treatment or prevention of erectile dysfunction which comprises administering to a mammal in need of such treatment or prevention an effective amount of a compound of claim 1.

22. A method for modulating appetite and metabolic rates of mammals which comprises administering to a mammal in need of such treatment or prevention an effective amount of a compound of claim 1.

23. A method for treating or preventing disorders that cause reduction in appetite, feeding behavior and/or body weight in a mammal which comprises administering to a mammal in need of such treatment or prevention an effective amount of a compound of claim 1.

24. A method for acutely stimulating the appetite of companion animals for the treatment of hepatic lipidosis, cachexia and other pathologies resulting in/from inappropriate food intake and weight loss, which comprises administering to a mammal in need of such treatment or prevention an effective amount of a compound of claim 1.

25. A method for acutely stimulating the appetite of livestock for the treatment of ketosis, postpartum anestrus, and other metabolic and reproductive pathologies resulting in/from inappropriate food intake and weight loss which comprises administering to a mammal in need of such treatment or prevention an effective amount of a compound of claim 1.

26. A method that will enhance growth and survivability of neonates in livestock which comprises administering to a mammal in need of such treatment or prevention an effective amount of a compound of claim 1.

27. A pharmaceutical composition which comprises a compound of claim 1 and a pharmaceutically acceptable carrier.

28. A pharmaceutical composition of claim 27 further comprising a second active ingredient selected from an insulin sensitizer, insulin mimetic, sulfonylurea, α-glucosidase inhibitor, HMG-CoA reductase inhibitor, sequestrant cholesterol lowering agent, β3 adrenergic receptor agonists, neuropeptide Y antagonist, phosphodiester V inhibitor, and α-2 adrenergic receptor antagonist.
